(19) United States
(12) Patent Application Publication
(10) Pub. No.: US 2026/0130766 A1
(43) Pub. Date: May 14, 2026

(54) MOLDS FOR SETTABLE MULTI-COMPONENT COMPOSITIONS AND METHOD OF FORMING SAID COMPOSITIONS

(71) Applicant: Abyrx, Inc., Stamford, CT (US)

(72) Inventors: Aniq DARR, Riverdale, NY (US); John PACIFICO, Greenwich, CT (US)

(21) Appl. No.: 19/114,276

(22) PCT Filed: Sep. 22, 2023
(86) PCT No.: PCT/US2023/074901

§ 371 Date: Mar. 21, 2025

Related U.S. Application Data
(60) Provisional application No. 63/376,689, filed on Sep. 22, 2022.

(51) Int. Cl. A61F 2/30

(52) U.S. Cl. CPC A61F 2/30942; A61F 2002/30062; A61F 2002/30957; A61F 2002/3096

(57) ABSTRACT

The present disclosure provides a method of forming a molded composition for bone repair or reconstruction. The method includes determining an implant shape based on a shape of an implant site of a patient and a surgical procedure being performed on the patient selecting a mold based on the implant shape, molding a settable composition using the mold to form a molded composition, and implanting the molded composition in the patient. The settable composition may be formed by combining at least two components.

### RELATED APPLICATIONS

This application claims priority to, and the benefit of, U.S. Provisional Application No. 63/376,689, filed Sep. 22, 2022, the contents of which are incorporated herein by reference in their entireties.

### TECHNICAL FIELD

The present disclosure relates to molds for molding absorbable and non-absorbable compositions into implants used in bone repair or reconstruction. Also provided herein are methods for using the molds to form implants for use in surgical application to repair gaps and/or fractures or to provide tissue adhesion.

### BACKGROUND

Formable putties are used during surgical procedures to form implants for bone repair and reconstruction. Current methods include pre-forming implants of pre-determined sizes and stocking the implants to meet potential sizing needs. Pre-formed implants may encounter manufacturing and/or supply chain issues and may not match the sizing required by the procedure. Custom sized implants may be determined by imaging (e.g., CT, MRI, etc.) or, in situations where imaging does not show the desired details, an initial medical procedure. The custom sized implant must then be manufactured (e.g., molded, cast, 3D printed, etc.) and subsequently implanted, which may take a substantial amount of time and add an additional medical procedure delaying patient recovery time, and increasing medical costs. Thus, there is a need to improve surgical procedures involving implants to increase the efficiency of forming implants.

### SUMMARY

In some embodiments, a method of forming a molded composition for bone repair or reconstruction includes determining an implant shape based on a shape of an implant or defect site of a patient and a surgical procedure being performed on the patient selecting a mold based on the implant shape, molding a settable composition using the mold to form a molded composition, and implanting the molded composition in the patient.

In some embodiments, a molded composition for bone repair is formed by a process that includes the steps of determining implant shape based on a shape of an implant or defect site of a patient and a surgical procedure being performed on the patient, selecting a mold based on the implant shape, molding a settable composition using the mold to form a molded composition, and implanting the molded composition in the patient.

In some embodiments, a kit for use in tissue repair or reconstruction includes a settable composition, and at least one mold structured to mold the settable composition into a molded composition having a shape corresponding to a shape of an implant site of a patient.

### BRIEF DESCRIPTION OF DRAWINGS

The drawings accompanying and forming part of this specification are included to depict certain aspects of the disclosure. It should be noted that the features depicted in the drawings are not necessarily drawn to scale. A more complete understanding of the disclosure and the advantages thereof may be acquired by referring to the following description, taken in conjunction with the accompanying drawings, in which like reference numbers indicate like features and wherein:

FIG. 1 is a flowchart of a method for molding and implanting a molded composition in a patient, according to an embodiment.

FIGS. 2A-2F depict the process of forming and implanting a cone shaped implant, according to an exemplary embodiment.

FIGS. 3A-3C depict the process of forming a spherical implant, according to an exemplary embodiment.

FIGS. 4A-4C depict the process of forming a half-spherical implant, according to an exemplary embodiment.

FIGS. 5A-5C depict the process of forming a cylindrical implant, according to an exemplary embodiment.

FIGS. 6A-6C depict the process of forming a crescent moon shaped implant, according to an exemplary embodiment.

FIGS. 7A-7C depict the process of forming a rectangular implant, according to an exemplary embodiment.

FIGS. 8A-8C depict the process of forming a boat-shaped implant, according to an exemplary embodiment.

FIGS. 9A-9B depict the process of forming a high aspect ratio cylindrical implant, according to an exemplary embodiment.

FIGS. 10A-10C depict the process of forming a half-moon shaped implant, according to an exemplary embodiment.

FIGS. 11A-11C depict the process of forming a circular implant, according to an exemplary embodiment.

FIGS. 12A-12C depict the process of forming a ring shaped implant, according to an exemplary embodiment.

FIGS. 13A-13C depict the process of forming a dome shaped implant, according to an exemplary embodiment.

FIGS. 14A-14C depict the process of forming a square shaped implant, according to an exemplary embodiment.

FIG. 15 is a block diagram for a kit including components for forming settable compositions that can be implanted, and molds for molding the settable compositions into a desired shape corresponding to an implant site, according to an exemplary embodiment.

### DETAILED DESCRIPTION

Embodiments herein relate generally to devices, methods, and kits for forming compositions into implants for medical procedures. Particularly, the devices, methods, and kits described herein are configured to allow for a medical professional, during a surgical procedure, to form an implant from a moldable composition. The embodiments described herein include molds that are configured to form settable compositions for as implants during surgical procedures. The molds described herein are configured to be available to medical professionals during a surgical procedure so that the implant shape can be determined and the implant may be formed during the same surgical procedure.

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “approximately” or “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

As used herein, the terms “patient” or “subject” are used interchangeably herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats, and horses. Preferred agricultural animals would be pigs, cattle, and goats.

The phrases “therapeutically effective amount” and “effective amount” and the like, as used herein, indicate an amount necessary to administer to a patient, or to a cell, tissue, or organ of a patient, to achieve a therapeutic effect, such as an ameliorating or, alternatively, a curative effect. The effective amount is sufficient to elicit the biological or medical response of a cell, tissue, system, animal, or human that is being sought by a researcher, veterinarian, medical doctor, or clinician. Determination of the appropriate effective amount or therapeutically effective amount is within the routine level of skill in the art.

The term “composition,” may, in some embodiments, may refer to putties. The term “putty” and the likes refer to soft, moldable, cohesive compositions, as used herein refers to most often formed viscous suspensions or viscoelastic composites (i.e., dispersions of particles in a viscous fluid). The present invention may also be formed from monolithic compositions of waxes, soft polymers, and other settable compositions.

Surgical procedures (e.g., osteotomies, revisions, bone grafts, etc.) may require implants (e.g., objects inserted into the body) to repair, mechanically support, or enhance damaged or missing biological structure (e.g., bones, cartilage, etc.) Certain procedures allow for the use of putties (e.g., settable, non-absorbable, absorbable, etc.) in forming the implant. Many of the procedures that allow for the use of settable compositions are standard (e.g., repeating) procedures that have similarly shaped implants, resulting in medical professionals (e.g., surgeons, surgical assistants, nurses, etc.) repeating the same implant formation process. Surgical procedures may include implanting implants within a body of a patient such as, for example, plates, pins, rods, hardware (e.g., screws, bolts, etc.) that are made of settable compositions. In some embodiments, implants may those formed solely from settable compositions, or may include a combination of implant formed from settable compositions and traditional (e.g., metal, etc.) implants.

Any of the aspects and embodiments described herein can be combined with any other aspect or embodiment as disclosed here in the Summary of the Invention, in the Drawings, and/or in the Detailed Description of the Invention, including the below specific, non-limiting examples/embodiments of the present invention.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to, or equivalent to, those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and are not intended to be limiting.

Other features and advantages of the application will become apparent from the following detailed description in conjunction with the examples.

Various embodiments described herein for forming implants using molds may provide one or more benefits including, for example: (1) reducing the number of surgical procedures to shape an implant into a desired shape and position the implant in a patient by allowing for a medical professional to determine an implant shape based on the implant site and forming an implant having the implant shape during a single surgical procedure; (2) reducing surgery time by allowing a surgeon to mold settable compositions into a desired shape using a simple molding process; (3) increasing the consistency of implants by providing a reusable mold; (4) decreasing the likelihood of error resulting from repeat operations; (5) mitigating supply chain and/or manufacturing issues associated with pre-manufactured implants; (6) mitigating size matching issues associated with pre-manufactured implants; (7) decreasing the burden on a hospital for stocking the correct size implants; and (8) reducing the delay between determining the implant size, implant type and the implantation procedure.

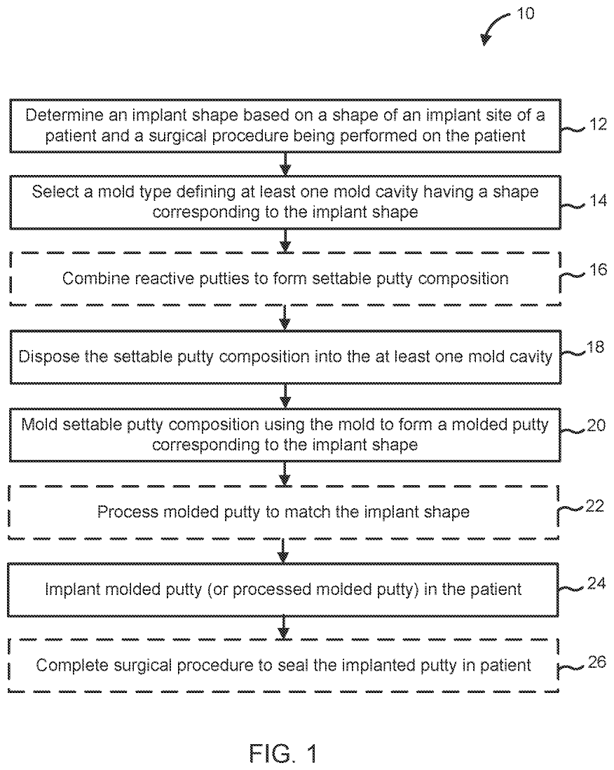

FIG. 1 is a flowchart of a method 10 for molding and implanting a settable composition in a patient, according to an embodiment. The method 10 may be completed by or in any combination by a medical professional (e.g., by a surgeon or nurse) manually, via hand tools (e.g., scalpels, shaping tools, etc.), and/or via electronic tools (e.g., computing devices, robotic systems, etc.), for example, during a surgical procedure being performed on the patient, as described herein.

The methods include determining an implant shape based on a shape of an implant site of a patient and a surgical procedure being performed on the patient, at 12. The implant site may be any location on a human body (e.g., foot, ankle, knee, hip, rib, spine, neck, head, etc.) or an animal body in which an implant is desirable for bone repair or reconstruction. In some embodiments, additional parameters may be considered when determining the implant shape, such as patient age, previous procedures, other medical conditions, etc. In some embodiments, the implant shape is determined directly by a medical professional based on prior experience. In some embodiments, the implant shape is determined by a medical professional using a reference source (e.g., reference table, textbook, etc.). In some embodiments, a computing device (e.g., scanning device, imaging device, etc.) is utilized to determine the implant shape. For example, a number of parameters (e.g., implant site, surgical procedure, patient age, etc.) may be input into the computing device and the computing device may determine the implant shape based on the number of parameters. The computing device may utilize an algorithm (e.g., search algorithm, artificial intelligence, etc.), lookup table, or similar function to determine the implant shape. In some embodiments, the implant shape may be determined visually by the surgeon during the surgical procedure. For example, once the surgeon cuts or separates epithelial and muscular tissue at the implant site to expose the implant site on a bone tissue (e.g., a site that may have ruptured, cracked, broken, split, or otherwise needs an implant), the surgeon may be able to visualize the shape of the implant site and determine based on at least the visual identification a desired implant shape.

For example, the implant shape may be determined to be a triangular cone, a sphere, half sphere, cylindrical cone, cylindrical, crescent moon shaped, 3D crescent moon shaped, rectangular, elongated, cube, half-moon shaped, circular, dome, or any other shape that may be used in a surgical procedure. In some embodiments, the implant shape may be determined to be a modified version of a shape. For example, the implant shape may generally be a cone shape with custom shaped portions that are specific to the patient's anatomy and needs of the surgical procedure (e.g., an implant site which generally has symmetric shape, but has asymmetric edges or portions that deviate from the overall symmetric shape). In some embodiments, the implant shape may be a combination of shapes. For example, the implant shape may include a first portion having a first shape (e.g., triangular cone, a sphere, half sphere, cylindrical cone, cylindrical, crescent moon shaped, 3D crescent moon shaped, rectangular, elongated, cube, half-moon shaped, circular, dome shape, etc.) and at least a second portion having a second shape different from the first shape.

At 14, a mold type is selected. The mold type corresponds to a mold defining at least one mold cavity having a shape corresponding to the implant shape. In some embodiments, multiple molds may be selected. For example, if the implant shape includes a cylindrical portion and a cone portion, a cylindrical mold and a cone mold are selected. In some embodiments, the mold type is the same shape as the implant shape. In some embodiments, the mold type is roughly or approximately the same shape as the implant shape. For example, the implant shape may be generally circular, hemispherical, conical rectangular, polygonal, etc., but may also include irregularities or asymmetries that cause the implant shape to deviate from its general shape that the selected mold does not account for.

Selecting a mold type may include selecting a mold based on a number of mold characteristics, for example, mold material, mold finish, mold shape, mold size, mold cavity shape, number of mold cavities, and the like. The mold may be formed of a material that allows for the composition to be formed accurately. In some embodiments, the mold may be flexible (e.g., formed of silicon, etc.) to allow for the composition to be more easily removed after molding, for example, by pressing on a back surface of the mold to push out the molded composition. In some embodiments, the material of the mold is non-reactive (e.g., non-stick, etc.) with the composition. The mold or at least the mold cavity may be finished so as to have a surface texture to allow for the composition to be released or removed from the mold. For example, at least the mold cavity may be finished to have a smooth surface, or to have a micro or nanotexture that inhibits the composition from sticking to the mold, thereby facilitating removal of the molded composition from the mold cavity. In some embodiments, the mold may optionally include a lining formed of a non-reactive material or at least the mold cavity coated with a non-reactive material (e.g., silicon, TEFLON®, silane, wax, oils, a hydrophobic material, a hydrophilic material, or any other suitable non-reactive material, etc.) that inhibits the composition from sticking to the mold cavity, thereby facilitating removal of the molded composition from the mold.

The mold may have more than one cavity (e.g., a plurality of mold cavities) having the same shape or different shapes to allow for more than one molded composition to be formed. For example, the mold may include a plurality of cube shaped cavities, or two cube shaped cavities and a triangular cavity. As another example, the mold may have a first cavity corresponding to a first portion of an implant (e.g., an implant left half) and a second cavity corresponding to and a second portion of the implant (e.g., an implant right half). In some embodiments, the mold includes a top portion and a bottom portion that, when compressed, mold the composition into the cavities. Both the top portion and the bottom portion may include at least one cavity, or just one of the portions may include at least one cavity. In some embodiments, the mold itself may be a device such as an extruder that may form the composition into elongated shapes (e.g., strands, strings, or high aspect ratio cylindrical shapes).

In some embodiment, the method 10 may optionally, also include combining components (e.g., reactive components) to form a settable composition. In some embodiments, the method 10 skips to operation 16 and continues directly to operation 18. For example, if the settable composition is a single ingredient composition, the single ingredient composition can be directly molded without mixing it with any other component. The components only set when combined together into the settable composition, which allows for the components to be stored separately and be prepared directly in the operating room to maximize working time for the composition.

In some embodiments, the components, when combined together, form an absorbable settable composition. In some embodiments, absorbable compositions are formed by the reaction of one or more polyaromatic di- or polyisocyanates with one or more diols or polyols and/or polyamines. The process for forming the polyurethane and polyureaurethane compositions of the invention may also include the addition of an optional chain extender or crosslinker. In one embodiment, the compositions of the invention are formed in the absence of a crosslinker. In one embodiment, the composition is formed by a process of combining a polyol and/or a polyamine, a polyaromatic di- or poly-isocyanate, and a carboxylic acid. In one embodiment, the carboxylic acid is selected from benzoic acid, malic acid, and succinic acid. In another embodiment, the composition is formed by a process of combining a polyol and/or polyamine, a polyaromatic polyisocyanate, and water.

In some embodiments, the components, when combined together, form a non-absorbable settable composition. In some embodiments, the components include a first component, component A, and a second component, component B, which can be hand mixed. The components include amounts of reagents which react and cure into a final, hardened form selected from the group consisting of polyurethane, polyureaurethane, polyetherurethane and polyetherureaurethane, over a period of time at room or body temperature, each component being physically separated from the other component of the composition; wherein component A includes a polyisocyanate component, a polyol or a polyamine component, a non-hydrolysable crosslinker, a particulate material, and additive material(s); and wherein component B includes a polyisocyanate component, a polyol and/or a polyamine component, a nonabsorbable cross-linker, a particulate material, and additive material(s). In some embodiments, the settable, nonabsorbable polyurethane, polyureaurethane, polyetherurethane or polyetherureaurethane compositions of the present disclosure, are formed by the reaction of one or more polyaromatic di- or polyisocyanates with one or more diols or polyols and/or diamines or polyamines. In some embodiments, the settable, nonabsorbable polyurethane, polyureaurethane, polyetherurethane or polyetherureaurethane compositions of the present disclosure, the compositions of the invention may also include the addition of an optional chain extender or crosslinker. In some embodiments, the settable, nonabsorbable polyurethane, polyureaurethane, polyetherurethane or polyetherureaurethane compositions are formed in the absence of a crosslinker. Examples of non-absorbable putties that can be used in the method 10 or with any of the molds described herein can be found in PCT Application No. PCT/US2022/038801, filed Jul. 29, 2022, published as WO 2023/014592, and entitled “Nonabsorbable Settable Multi-Putty Cements, Homeostatic Compositions, and Methods of Use,” which is incorporated reference herein in its entirety.

At 18, the settable composition is disposed in the at least one mold cavity of the mold. In some embodiments, a releasing agent (e.g., TEFLON®, silane, wax, oils, a hydrophobic material, a hydrophilic material, or any other suitable non-reactive material, etc.) may, optionally, be applied to the mold, to prevent the composition from sticking, prior to disposing the settable composition. For example, the mold may be provided with the releasing agent already disposed on a surface of at least the mold cavity, or the medical provider may coat the releasing agent (e.g., spray coat or dip coat) the releasing agent on the mold prior to disposing the settable composition in the one or more mold cavities. In some embodiments, the mold may be provided with corresponding information (e.g., an operation manual or standard operating procedure documents) regarding how much of the settable composition should be disposed within the cavity. For example, a first mold may be configured or structured to receive a first amount of the settable composition and a second mold may be configured or structured to receive a second amount of the settable composition. In some embodiments, the mold is configured to accept excess composition. In such embodiments, the excess composition material protrudes outwards from the mold cavity as flash as the settable composition is molded, which may be removed. The mold may include protrusions that result in a perforated flash to aid in removal. In some embodiments, the composition disposed into the at least one mold cavity may be pre-shaped (e.g., shaped into a shape that is roughly similar to the cavity) by a medical professional, or may be disposed in the at least one mold cavity in any form.

At 20, the settable composition is molded using the mold to form a molded composition corresponding to the implant shape. In some embodiments, molding the settable composition includes squeezing two halves of a mold together. The two halves may be squeezed together until a predetermined point, for example, by application of a predetermined force until the two halves are fully in contact, until the two halves of the mold cannot be brought any closer to each other by application of the predetermined force, until there is a certain flash thickness (e.g., when excess composition is disposed in the mold cavity), or the like. In some embodiments, molding the settable composition includes operating a device, such as an extruder including a mold cavity through which the settable composition is extruded to form the molded composition. The device may be operated until the desired length of molded composition is extruded. After the settable composition is molded, the molded composition is removed from the mold. Removing the molded composition may be done by hand (e.g., via gravity, pulling, manipulating the mold, etc.) or by using a tool to facilitate molded composition removal.

In some embodiments, the molded composition is optionally, processed to match the implant shape, at 22. In some embodiments, the method 10 may skip operation 22 and continue right to operation 24. For example, if the molded composition removed from the mold matches the implant shape, processing is not performed. In some embodiments, processing the molded composition may include joining pieces of molded composition together. Joining may include at least one of scoring, adhering via adhesive, bonding, fusing, or a similar process to aid in joining pieces of molded composition together. In some embodiments, pieces of molded composition may be coupled, connected, or joined with each other without additional processing. In some embodiments, coupling the molded composition may include lining up corresponding guides to ensure accurate coupling. In some embodiments, processing the molded composition may include removing flash or similar material formed as a byproduct of the molding step from the molded composition. The flash may be removed with a tool (e.g., knife, scalpel, etc.) or manually. In some embodiments, the flash may be perforated during molding, allowing for it to be torn away. In some embodiments, processing the molded composition includes reshaping (e.g., resizing, removing, reconfiguring, etc.) at least a portion of the molded composition. For example, if the implant shape was determined to have divots, indents, detents, and/or protrusions, or otherwise deviations from the general shape of the mold, the divots and/or protrusions may be formed on the molded composition to match the implant shape. In some embodiments, a portion of the molded composition may be removed to match the implant shape. In some embodiments, additional composition may be added to the molded composition to match the implant shape. In some embodiments, processing the molded composition may include a plurality of various processes. In some embodiments, tools (e.g., carver, probe, spatula, chisel, hollow tip, pick, etc.) are used to process the molded composition.

At 24, the molded composition (e.g., the processed molded composition) is implanted in the patient. For example, a medical professional implants (e.g., inserts, disposes, presses, etc.) the molded composition into the implant site of the patient. In some embodiments, multiple molded compositions may be implanted into the patient. In some embodiments, the molded composition may be additionally processed after implantation. For example, the molded composition may need to be pressed into bone around the implant site to form a smooth seal. In some embodiments, the implant site may, optionally, be prepared with an adhesive (e.g., glue, bone cement, etc.) or a coating (e.g., polyurethane, polyureaurethane, etc.) prior to implanting the molded putty to improve adhesion between the implant and the implant site. In some embodiments, implantation of the molded composition occurs after the molded composition or processed molded composition has completely hardened (e.g., has a bone like hardness). In some embodiment, the molded composition or the processed molded composition is implanted before the molded composition has completely hardened, for example, is still soft or malleable. This may facilitate implantation by allowing the molded composition or processed molded composition to flex and conform to contours of the implant site on implantation. In some embodiments, the molded composition may be formulated to have a sufficiently long hardening time to allow sufficient time for the medical professional to implant the molded composition at the implant site while it is still hardening. In some embodiments, a hardening time of the molded composition or the processed molded composition may be in a range of about 2 minutes to about 1 hours, inclusive.

At step 26, the surgical procedure is completed to seal the implanted composition in the patient. In some embodiments, additional procedures may need to occur prior to completing the implantation procedure. Once the composition is implanted in the patient, the composition may set (e.g., harden) and forms a rigid implant.

Any suitable shaped or sized mold may be used to form a molded implant having a desired shape or size. For example, a medical professional or a medical office may have a number of molds available during a surgical procedure where molding a settable composition to correspond to an implant shape is needed. As discussed in reference to operation 14 of FIG. 1, molds may be designed, configured, and/or selected to match the surgical procedure and the implant shape. Various embodiments of the molds described herein may be formed of a variety of materials (e.g., silicone, plastic, polymers, metals, alloys, etc.) and material types (e.g., flexible, rigid, semi-rigid, etc.) to match what is desirable for the particular application. Certain surgical procedures may require multiple molds to be used to form all the components required to form the implant. In addition to the various molds described herein, additional molds configured to form other shapes, sizes, and number of molded putties may be available to a medical professional. Furthermore, certain molds may be reconfigured and combined to form molded compositions with a desired shape.

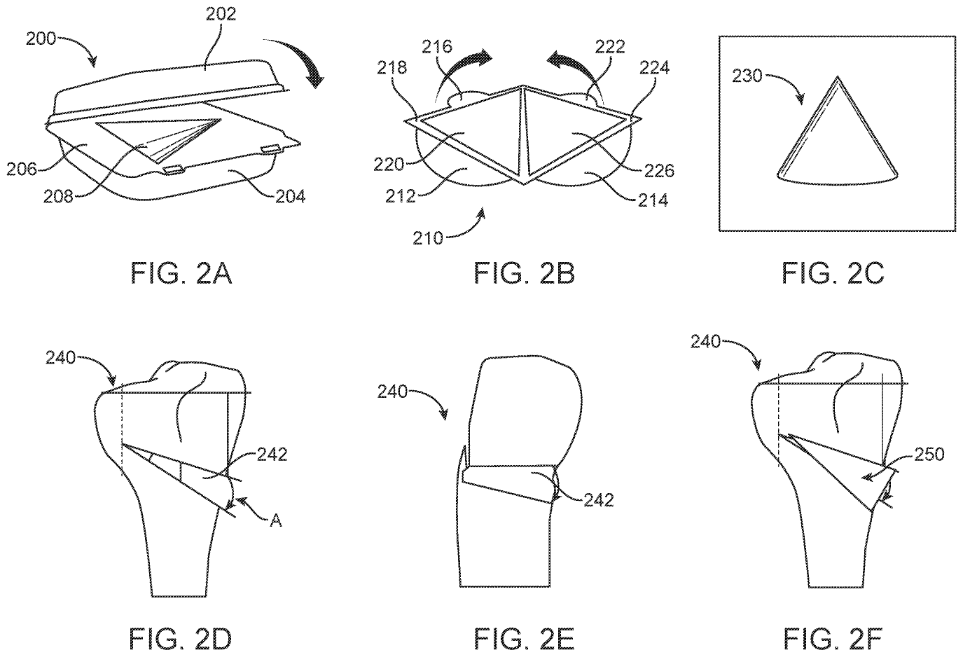

FIGS. 2A-2F depict the process of forming and implanting a cone implant using a mold 200 having a conical shaped mold cavity 208, according to an exemplary embodiment. Cone implants may include triangular cone implants, which may be desired for surgical procedures such as high tibial osteotomies. Cone implants may also include cylindrical cone implants, which may be desired for surgical procedures such as proximal humerus work, where the cylindrical cone implant acts as a strut. In some embodiments, cone implants may be formed to have base dimensions (e.g., length, width, diameter, etc.) in a range of about 7 mm to about 12 mm, inclusive or about 10 mm to about 15 mm, inclusive, and a height in a range of about 2 inches to about 4 inches, inclusive.

Expanding further, FIG. 2A depicts a perspective view of the mold 200 including a top portion 202 and a bottom portion 204. In some embodiments, the top portion 202 and the bottom portion 204 are coupled (e.g., via a fastener, hinge, etc.) that allows for the mold 200 to be opened, to allow for unmolded composition (e.g., a settable composition) to be disposed within, and closed, to allow for molding of the composition. The bottom portion 204 includes a flat surface 206 and a mold cavity 208 extending away from the flat surface 206 and into the body of the bottom portion 204. The shape of the mold cavity 208 has a conical shape configured to mold a settable composition into a molded putty having a conical shape. In some embodiments, the top portion 202 also includes a mold cavity also defining at least a portion of a conical shape. In some embodiments, the mold cavity 208 and the cavity located in the top portion 202 are aligned to mold the composition in conjunction with each other. In some embodiments, the top portion 202 only includes a flat surface configured to press the settable composition into the mold cavity 208 of the bottom portion 204. In some embodiments, the mold cavity 208 may include features that allow for the molded composition to include attributes such as flash, guide tabs, or the like, when molded.

FIG. 2B depicts a perspective view of an unprocessed composition 210 including a first composition half 212 and a second composition half 214, which may be formed using the mold 200. In some embodiments, the unprocessed composition 210 may include additional pieces. “Unprocessed” refers to composition that has been formed by the mold but has not yet been subjected to any further operation after being molded (e.g., flash has not been removed, have not been joined or adhered with another piece, have not yet been further shaped into the implant shape). Both the first composition half 212 and the second composition half 214 are formed by the mold 200 of FIG. 2A. In some embodiments, the first composition half 212 and the second composition half 214 are formed together during one molding operation or are formed during separate molding operations. The first composition half 212 includes a guide tab 216 and flash 218. Both the guide tab 216 and the flash 218 are remnants of the molding process. In some embodiments, the first composition half 212 may not include one or both of the guide tab 216 and the flash 218. The first composition half 212 also includes a flat surface 220 which serves as a coupling point between the first composition half 212 and the second composition half 214. In some embodiments, the flat surface 220 may be prepared (e.g., scored, marked, etc.) prior to coupling the first composition half 212 and the second composition half 214. The second composition half 214 includes a guide tab 222 and flash 224, which correspond to the guide tab 216 and the flash 218 and are substantially similar. In some embodiments, the second composition half 214 may not include one or both of the guide tab 222 and the flash 224. The second composition half 214 includes as flat surface 226 which corresponds to and couples to the flat surface 220. Once the first composition half 212 and the second composition half 214 are joined together at the flat surfaces (220 and 226), the flash (218 and 224) and the guide tabs (216 and 222) may be removed during processing to prepare the implant for implantation.

FIG. 2C depicts a side view of a molded composition 230 molded using the mold 200 (e.g., after forming and processing the unprocessed composition 210). The molded composition 230 has a conical shape and is prepared for implantation during the surgical procedure and/or additional processing by a medical professional. In some embodiments, the molded composition 230 may be just one of several molded putties needed to form the final implant shape. In some embodiments, the molded composition 230 is formed by coupling together the first composition half 212 and the second composition half 214.

FIG. 2D depicts a side view of a tibia 240 having a tissue cavity 242 having a general conical shape. The cavity 242 may have been formed by removal of bone tissue during the surgical procedure, or a result of an injury to the patient, and needs to be supplemented with an implant. A medical professional may take measurements of the dimensions of the tissue cavity 242, such as the length, width, depth, angle A, and the like, to determine the implant shape. FIG. 2E depicts a front view of the tibia 240 having the tissue cavity 242. FIG. 2F depicts a side view of the tibia 240 with an implant 250 implanted in the tissue cavity 242. The implant 250 may be substantially similar to the implant 230 and has dimensions configured to match the dimensions of the tissue cavity 242. Measuring dimension, forming the molded composition, and implanting all take place during once surgical procedure to reduce the number of procedures for both the patient and the medical professionals involved.

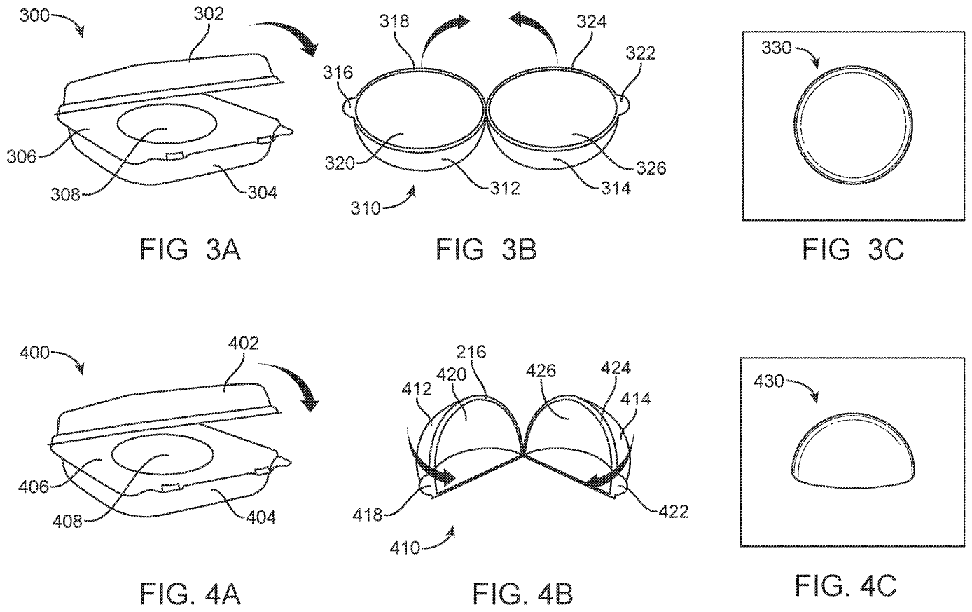

FIGS. 3A-3C depict the process of forming a spherical implant 330 using a mold 300, according to an exemplary embodiment. Spherical implants may be desired for surgical procedures involving acetabulums and glenoids. In some embodiments, spherical implants may have a first dimension ranging from about 9 mm to about 13 mm, inclusive, with a mean of about 10 mm, a second dimension ranging from about 11 mm to about 19 mm, inclusive, with a mean of about 15.50 mm, and a third dimension ranging from about 15 mm to about 25 mm, inclusive with a mean of about 20 mm.

FIG. 3A depicts a perspective view of the mold 300 including a top portion 302 and a bottom portion 304. In some embodiments, the top portion 302 and the bottom portion 304 are coupled (e.g., via a fastener, hinge, etc.) that allows for the mold 300 to be opened, to allow for unmolded composition to be disposed within, and closed, to allow for molding of the composition (e.g., a settable composition). The bottom portion 304 includes a flat surface 306 and a mold cavity 308 extending away from the flat surface 306 and into the body of the bottom portion 304. The mold cavity 308 has a spherical shape configured to form one half of the spherical molded composition 330. In some embodiments, the top portion 302 also includes a mold cavity having a spherical shape configured to form a second half of the spherical molded composition 330. In some embodiments, the mold cavity 308 and a cavity located in the top portion 302 are aligned to mold composition in conjunction. In some embodiments, the top portion 302 only includes a flat surface configured to press the settable composition into the cavity of the bottom portion 304. In such embodiments, two dome shaped portions may be formed using the mold 300 that may be then coupled together to form a spherical shape molded composition 330. In some embodiments, the mold cavity 308 may include features that allow for the molded composition to include attributes such as flash, guide tabs, or the like, when molded.

FIG. 3B depicts a perspective view of an unprocessed composition 310 including a first composition half 312 and a second composition half 314, which may be formed using the mold 300. In some embodiments, the unprocessed composition 310 may include additional pieces. Both the first composition half 312 and the second composition half 314 are formed by the mold 300 of FIG. 3A. In some embodiments, the first composition half 312 and the second composition half 314 are formed together during one molding operation or are formed during separate molding operations. The first composition half 312 includes a guide tab 316 and flash 318. Both the guide tab 316 and the flash 318 are remnants of the molding process. In some embodiments, the first composition half 312 may not include one or both of the guide tab 316 and the flash 318. The first composition half 312 also includes a flat surface 320 which serves as a coupling point between the first composition half 312 and the second composition half 314. In some embodiments, the flat surface 320 may be prepared (e.g., scored, marked, etc.) prior to coupling the first composition half 312 and the second composition half 314. The second composition half 314 includes a guide tab 322 and flash 324, which correspond to the guide tab 316 and the flash 318 and are substantially similar. In some embodiments, the second composition half 314 may not include one or both of the guide tab 322 and the flash 324. The second composition half 314 includes as flat surface 326 which corresponds to and couples to the flat surface 320. Once the first composition half 312 and the second composition half 314 are joined together at the flat surfaces (320 and 326), the flash (318 and 324) and the guide tabs (316 and 322) may be removed during processing to prepare the implant for implantation.

FIG. 3C depicts a side view of the molded composition 330 (e.g., after processing the unprocessed composition 310). The molded composition 330 has a spherical shape and is prepared for implantation during the surgical procedure and/or additional processing by a medical professional. In some embodiments, the molded composition 330 may be just one of several molded putties needed to form the final implant shape.

FIGS. 4A-4C depict the process of forming a half-spherical implant, according to an exemplary embodiment. Half-spherical implants may be desired for surgical procedures involving acetabulums and glenoids. In some embodiments, half-spherical implants may have a first dimension ranging from about 9 mm to about 13 mm, inclusive, with a mean of about 10 mm, a second dimension ranging from about 11 mm to about 19 mm, inclusive, with a mean of about 15 mm, and a third dimension ranging from about 15 mm to about 25 mm, inclusive with a mean of about 20 mm.

FIG. 4A depicts a perspective view of a mold 400 including a top portion 402 and a bottom portion 404. In some embodiments, the top portion 402 and the bottom portion 404 are coupled (e.g., via a fastener, hinge, etc.) that allows for the mold 400 to be opened, to allow for unmolded composition (e.g., a settable composition) to be disposed within, and closed, to allow for molding of the composition. The bottom portion 404 includes a flat surface 406 and a mold cavity 408 extending away from the flat surface 406 and into the body of the bottom portion 404. The shape of the mold cavity 408 has a hemispherical shape that directly corresponds to the desired shape and dimensions of the desired molded composition. In some embodiments, the top portion 402 also includes a mold cavity. In some embodiments, the mold cavity 408 and a mold cavity located in the top portion 402 are aligned to mold composition in conjunction with each other. In some embodiments, the top portion 402 only includes a flat surface configured to press the settable composition into the mold cavity 408 of the bottom portion 404. In some embodiments, the cavity 408 may include features that allow for the molded composition to include attributes such as flash, guide tabs, or the like, when molded.

FIG. 4B depicts a perspective view of an unprocessed composition 410 including a first composition half 412 and a second composition half 414, which may be formed using the mold 400. In some embodiments, the unprocessed composition 410 may include additional pieces. Both the first composition half 412 and the second composition half 414 are formed by the mold 400 of FIG. 4A. In some embodiments, the first composition half 412 and the second composition half 414 are formed together during one molding operation or are formed during separate molding operations. The first composition half 412 includes a guide tab 416 and flash 418. Both the guide tab 416 and the flash 418 are remnants of the molding process. In some embodiments, the first composition half 412 may not include one or both of the guide tab 416 and the flash 418. The first composition half 412 also includes a flat surface 420 which serves as a coupling point between the first composition half 412 and the second composition half 414. In some embodiments, the flat surface 420 may be prepared (e.g., scored, marked, etc.) prior to coupling the first composition half 412 and the second composition half 414. The second composition half 414 includes a guide tab 422 and flash 424, which correspond to the guide tab 416 and the flash 418 and are substantially similar. In some embodiments, the second composition half 414 may not include one or both of the guide tab 422 and the flash 424. The second composition half 414 includes as flat surface 426 which corresponds to and couples to the flat surface 420. Once the first composition half 412 and the second composition half 414 are joined together at the flat surfaces (420 and 426), the flash (418 and 424) and the guide tabs (416 and 422) may be removed during processing to prepare the implant for implantation.

FIG. 4C depicts a side view of a molded composition 430 formed using the mold 400 (e.g., after processing the unprocessed composition 410). The molded composition 430 has a half-spherical shape and is prepared for implantation during the surgical procedure and/or additional processing by a medical professional. In some embodiments, the molded composition 430 may be just one of several molded putties needed to form the final implant shape.

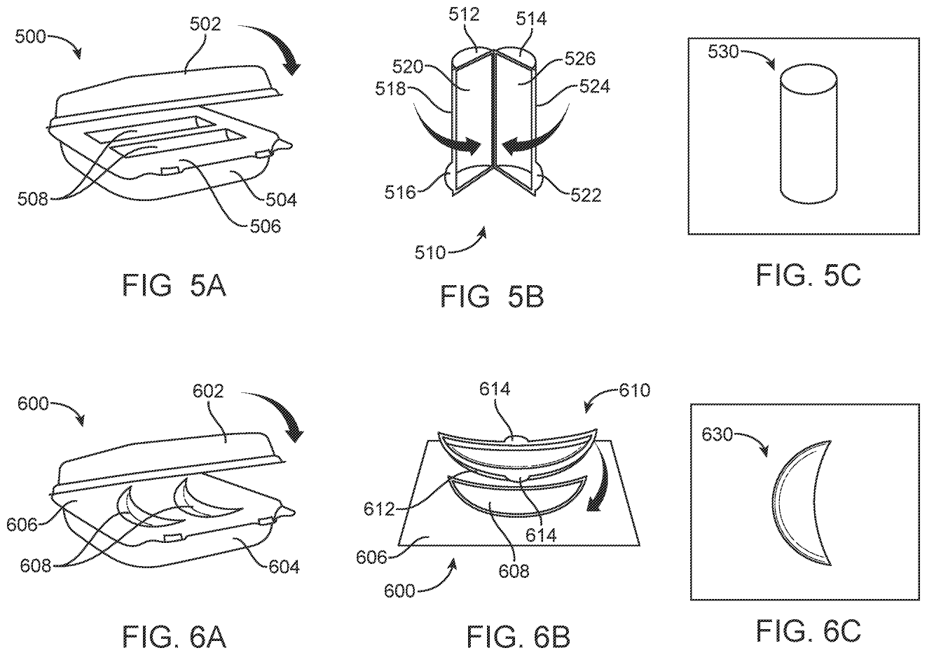

FIGS. 5A-5C depict a process of forming a cylindrical implant (e.g., bone dowel shape) using a mold 500, according to an exemplary embodiment. Cylindrical implants may be desired for surgical procedures such as ACL revision surgeries and for pin hole distractor pins. In some embodiments, cylindrical implants, such as those used as pins, may have a first dimension ranging from about 2.5 mm to about 4.5 mm, inclusive and a second dimension ranging from about 3.5 mm to about 5 mm, inclusive. In some embodiments, cylindrical implants, such as those used during ACL revision surgeries, may have a first dimension ranging from about 6 mm to about 8 mm, inclusive and a second dimension ranging from about 30 mm to about 50 mm, inclusive.

FIG. 5A depicts a perspective view of the mold 500. The mold 500 includes a top portion 502 and a bottom portion 504. In some embodiments, the top portion 502 and the bottom portion 504 are coupled (e.g., via a fastener, hinge, etc.) that allows for the mold 500 to be opened, to allow for unmolded composition (e.g., a settable composition) to be disposed within, and closed, to allow for molding of the composition. The bottom portion 504 includes a flat surface 506 and two mold cavities 508, each having a rectangular shape formed in the body of the bottom portion 504. Including two mold cavities 508 allows for two molded putties (e.g., implant halves, separate implants, etc.) to be formed simultaneously. While shown as including two mold cavities 508, in other embodiments, the mold 500 may include more than two mold cavities (e.g., 3, 4, 5, 6, or even more). The shape of the mold cavities 508 directly corresponds to the desired shape and dimensions of the desired molded composition. In some embodiments, the top portion 502 also includes a mold cavity. In some embodiments, the mol cavity 508 and a mold cavity located in the top portion 502 are aligned to mold composition in conjunction. In some embodiments, the top portion 502 only includes a flat surface configured to press the settable composition into the mold cavity of the bottom portion 504. In some embodiments, the mold cavities 508 may include features that allow for the molded composition to include attributes such as flash, guide tabs, or the like, when molded.

FIG. 5B depicts a perspective view of an unprocessed composition 510 including a first composition half 512 and a second composition half 514, which may be formed using the mold 500. In some embodiments, the unprocessed composition 510 may include additional pieces. Both the first composition half 512 and the second composition half 514 are formed by the mold of FIG. 5A and may be formed simultaneously in the two cavities 508. In some embodiments, the first composition half 512 and the second composition half 514 are formed together during one molding operation or are formed during separate molding operations. The first composition half 512 includes a guide tab 516 and flash 518. Both the guide tab 516 and the flash 518 are remnants of the molding process. In some embodiments, the first composition half 512 may not include one or both of the guide tab 516 and the flash 518. The first composition half 512 also includes a flat surface 520 which serves as a coupling point between the first composition half 512 and the second composition half 514. In some embodiments, the flat surface 520 may be prepared (e.g., scored, marked, etc.) prior to coupling the first composition half 512 and the second composition half 514. The second composition half 514 includes a guide tab 522 and flash 524, which correspond to the guide tab 516 and the flash 518 and are substantially similar. In some embodiments, the second composition half 514 may not include one or both of the guide tab 522 and the flash 524. The second composition half 514 includes a flat surface 526 which corresponds to and couples to the flat surface 520. Once the first composition half 512 and the second composition half 514 are joined together at the flat surfaces (520 and 526), the flash (518 and 524) and the guide tabs (516 and 522) may be removed during processing to prepare the implant for implantation.

FIG. 5C depicts a side view of a molded composition 530 formed using the mold 500 (e.g., after processing the unprocessed composition 510). The molded composition 530 has a cylindrical shape and is prepared for implantation during the surgical procedure and/or additional processing by a medical professional. In some embodiments, the molded composition 530 may be just one of several molded putties needed to form the final implant shape.

FIGS. 6A-6C depict the process of forming a crescent moon shaped implant using a mold 600, according to an exemplary embodiment. Crescent moon implants may be desired for surgical procedures such as those involving transforaminal lumbar interbody fusion cages. In some embodiments, crescent moon shaped implants have a first dimension ranging from about 5 mm to about 8 mm, inclusive a second dimension ranging from about 5 mm to about 10 mm, inclusive and a third dimension ranging from about 6 mm to about 10 mm, inclusive.

FIG. 6A depicts a perspective view of the mold 600 including a top portion 602 and a bottom portion 604. In some embodiments, the top portion 602 and the bottom portion 604 are coupled (e.g., via a fastener, hinge, etc.) that allows for the mold 600 to be opened, to allow for unmolded composition (e.g., a settable composition) to be disposed within, and closed, to allow for molding of the composition. The bottom portion 604 includes a flat surface 606 and two mold cavities 608, each having a crescent moon shape, formed in the body of the bottom portion 604. Including two mold cavities 608 allows for two molded putties (e.g., implant halves, separate implants, etc.) to be formed simultaneously. While shown as including two mold cavities 608, in other embodiments, the mold 600 may include more than two mold cavities (e.g., 3, 4, 5, 6, or even more). The shape of the mold cavities 608 directly corresponds to the desired shape and dimensions of the desired molded composition. In some embodiments, the top portion 602 also includes at last one mold cavity. In some embodiments, the mold cavities 608 and an at least one mold cavity located in the top portion 602 are aligned to mold composition in conjunction with each other. In some embodiments, the top portion 602 only includes a flat surface configured to press the settable composition into the mold cavity of the bottom portion 604. In some embodiments, the mold cavities 608 may include features that allow for the molded composition to include attributes such as flash, guide tabs, or the like, when molded.

FIG. 6B depicts a perspective view of an unprocessed composition 610 being removed from the mold cavity 608 of the mold 600. In some embodiments, the unprocessed composition 610 may include additional pieces. The unprocessed composition 610 was formed in the mold 600 of FIG. 6A and additional unprocessed putties may be molded simultaneous in the two mold cavities 608. The unprocessed composition 612 includes flash 612 and two guide tabs 614. Both the flash 612 and the guide tabs 614 are remnants of the molding process. In some embodiments, the unprocessed composition 610 does not include one or both of the flash 612 and the guide tabs 614. In some embodiments, the guide tabs 614 may be utilized to remove the unprocessed composition 610 from the mold cavity 608.

FIG. 6C depicts a side view of a molded composition 630 formed using the mold 600 (e.g., after processing the unprocessed composition 610). The molded composition 630 has a crescent moon shape and is prepared for implantation during the surgical procedure and/or additional processing by a medical professional. In some embodiments, the molded composition 630 may be just one of several molded putties needed to form the final implant shape.

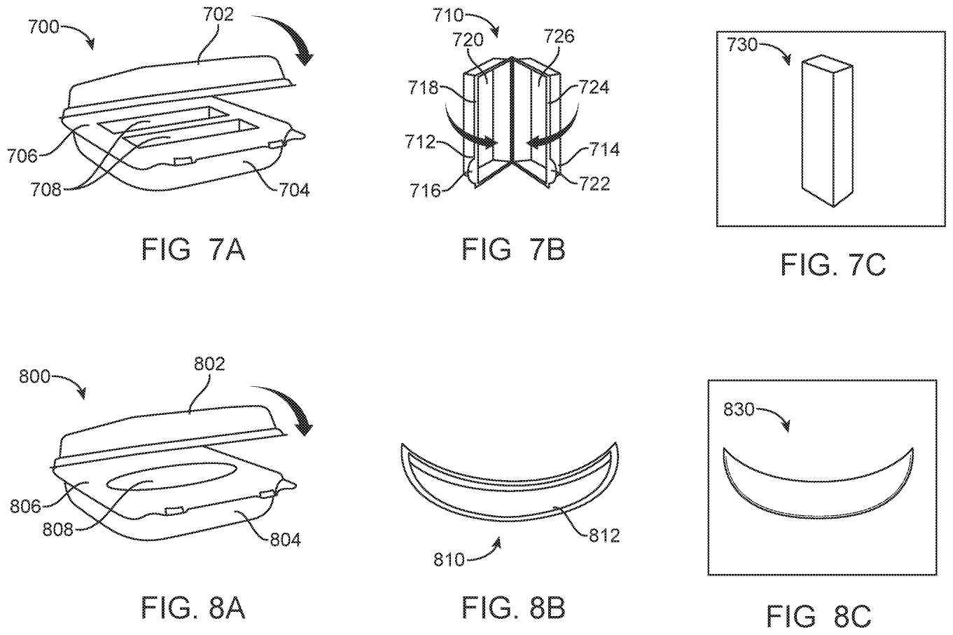

FIGS. 7A-7C depict the process of forming one or more rectangular implants using a mold 700, according to an exemplary embodiment. Rectangular implants may be desired for surgical procedures such as protecting the dura in the spine laid over posterior elements and iliac crest bone grafting. In some embodiments, the dimensions of the rectangular, rib-like, implants, such as those used to protect dura in the spine load over posterior elements correspond to the dimensions illustrated in Table 1. In some embodiments, rectangular, cube-like, implants with rounded tops used for iliac crest bone grafting have first dimensions between about 3 cm to about 6 cm, inclusive, second dimensions between about 3 cm to about 6 cm, inclusive, and third dimensions between about 2 cm to about 4 cm, inclusive.

TABLE 1
Rib Segment Dimensions

| | Length ± | | Depth ± | | Width ± | | |
|---|---|---|---|---|---|---|---|
| Rib | standard | | standard | | standard | | Volume |
| segment | error (mm) | | error (mm) | | error (mm) | | (mm³) |
| Rib 1 | 12.47 | 0.22 | 2.23 | 0.03 | 3.91 | 0.05 | 108.57 |
| Rib 2 | 14.78 | 0.25 | 2.02 | 0.03 | 2.66 | 0.04 | 79.59 |
| Rib 3 | 17.31 | 0.21 | 2.05 | 0.05 | 2.75 | 0.04 | 97.6 |
| Rib 11 | 7.96 | 0.55 | 0.93 | 0.05 | 1.92 | 0.08 | 14.28 |
| Rib 12 | 7.72 | 0.58 | 0.96 | 0.05 | 2.1 | 0.08 | 15.51 |
| Rib 13 | 7.27 | 0.39 | 0.89 | 0.05 | 1.93 | 0.05 | 12.5 |

FIG. 7A depicts a perspective view of the mold 700 including a top portion 702 and a bottom portion 704. In some embodiments, the top portion 702 and the bottom portion 704 are coupled (e.g., via a fastener, hinge, etc.) that allows for the mold 700 to be opened, to allow for unmolded composition (e.g., a settable composition) to be disposed within, and closed, to allow for molding of the composition. The bottom portion 704 includes a flat surface 706 and two mold cavities 708, each having a rectangular shape formed in the body of the bottom portion 704. Including two mold cavities 708 allows for two molded putties (e.g., implant halves, separate implants, etc.) to be formed simultaneously. While shown as including two mold cavities 708, in other embodiments, the mold 700 may include more than two mold cavities (e.g., 3, 4, 5, 6, or even more). The shape of the mold cavities 708 directly corresponds to the desired shape and dimensions of the desired molded composition. In some embodiments, the top portion 702 also includes a mold cavity. In some embodiments, the mold cavity 708 and one or more mold cavities located in the top portion 702 are aligned to mold composition in conjunction with each other. In some embodiments, the top portion 702 only includes a flat surface configured to press the settable composition into the mold cavity 708 of the bottom portion 704. In some embodiments, the mold cavities 708 may include features that allow for the molded composition to include attributes such as flash, guide tabs, or the like, when molded.

FIG. 7B depicts a perspective view of an unprocessed composition 710 including a first composition half 712 and a second composition half 714 formed using the mold 700. In some embodiments, the unprocessed composition 710 may include additional pieces. Both the first composition half 712 and the second composition half 714 are formed by the mold 700 of FIG. 7A and may be formed simultaneously in the two mold cavities 708. In some embodiments, the first composition half 712 and the second composition half 714 are formed together during one molding operation or are formed during separate molding operations. The first composition half 712 includes a guide tab 716 and flash 718. Both the guide tab 716 and the flash 718 are remnants of the molding process. In some embodiments, the first composition half 712 may not include one or both of the guide tab 716 and the flash 718. The first composition half 712 also includes a flat surface 720 which serves as a coupling point between the first composition half 712 and the second composition half 714. In some embodiments, the flat surface 720 may be prepared (e.g., scored, marked, etc.) prior to coupling the first composition half 712 and the second composition half 714. The second composition half 714 includes a guide tab 722 and flash 724, which correspond to the guide tab 716 and the flash 718 and are substantially similar. In some embodiments, the second composition half 714 may not include one or both of the guide tab 722 and the flash 724. The second composition half 714 includes as flat surface 726 which corresponds to and couples to the flat surface 720. Once the first composition half 712 and the second composition half 714 are joined together at the flat surfaces (720 and 726), the flash (718 and 724) and the guide tabs (716 and 722) may be removed during processing to prepare the implant for implantation.

FIG. 7C depicts a side view of a molded composition 730 using the mold 700 (e.g., after forming and processing the unprocessed composition 710). The molded composition 730 has a rectangular shape and is prepared for implantation during the surgical procedure and/or additional processing by a medical professional. In some embodiments, the molded composition 730 may be just one of several molded putties needed to form the final implant shape.

FIGS. 8A-8C depict the process of forming a boat-shaped implant using a mold 800, according to an exemplary embodiment. Boat-shaped implants may be desired for surgical procedures in which it may be desirable to tack down an autograft or allograft in the lateral gutters to avoid migration and to potentially aid in avoiding downstream procedures where the autograft or allograft may possibly migrate and get stuck on a nerve route causing post-operation pain for the patient. In some embodiments, the boat-shaped implants have a dimension (e.g., a length) in the range of about 20 mm to about 60 mm, inclusive.

FIG. 8A depicts a perspective view of the mold 800 including a top portion 802 and a bottom portion 804. In some embodiments, the top portion 802 and the bottom portion 804 are coupled (e.g., via a fastener, hinge, etc.) that allows for the mold 800 to be opened, to allow for unmolded composition to be disposed within, and closed, to allow for molding of the composition. The bottom portion 804 includes a flat surface 806 and two mold cavities 808 formed in the bottom portion 804. Including two mold cavities 808 allows for two molded putties (e.g., implant halves, separate implants, etc.) to be formed simultaneously. While shown as including two mold cavities 808, in other embodiments, the mold 800 may include more than two mold cavities (e.g., 3, 4, 5, 6, or even more). The shape of the mod cavities 808 directly corresponds to the desired shape and dimensions of the desired molded composition. In some embodiments, the top portion 802 also includes at last one mold cavity. In some embodiments, the mold cavities 808 and an at least one mold cavity located in the top portion 802 are aligned to mold composition in conjunction. In some embodiments, the top portion 802 only includes a flat surface configured to press the settable composition into the mold cavity 808 of the bottom portion 804. In some embodiments, the mold cavities 608 may include features that allow for the molded composition to include attributes such as flash, guide tabs, or the like, when molded.

FIG. 8B depicts a perspective view of an unprocessed molded composition 810 after removal from the mold 800. The unprocessed composition 810 includes flash 812 formed as a remnant of the molding process. FIG. 8C depicts a side view of a molded composition 830 after being processed to remove flash (e.g., after processing the unprocessed composition 810). The molded composition 820 in a boat-shaped and is prepared for implantation during the surgical procedure and/or additional processing by a medical professional. In some embodiments, the molded composition 820 may be just one of several molded putties needed to form the final implant shape.

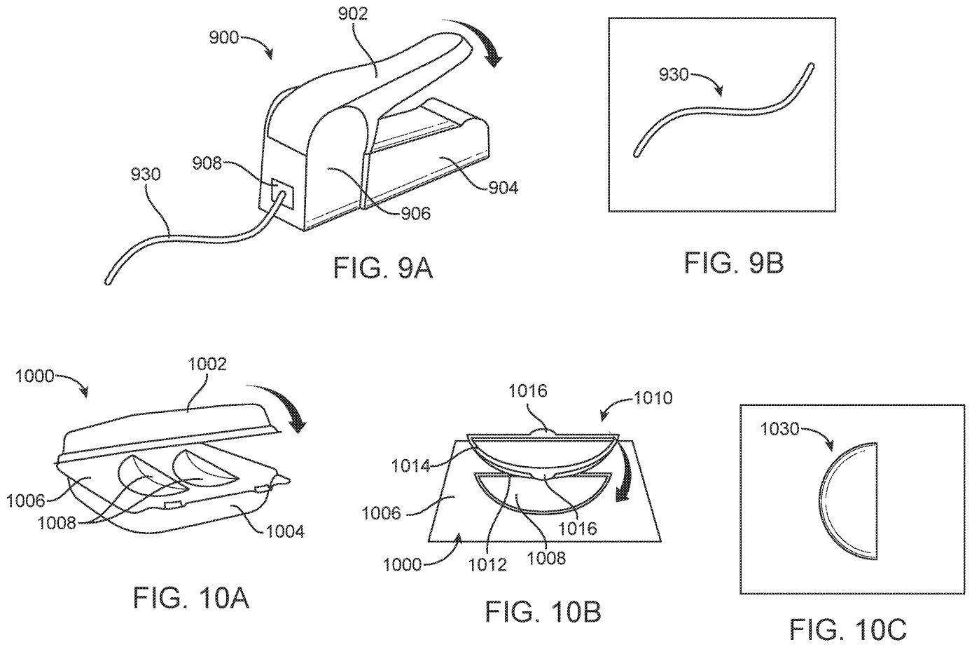

FIGS. 9A-9B depict the process of forming a high aspect ratio cylindrical implant (e.g., serpentine shaped, snake shaped, string shaped, etc.) using an extruder 900 used to mold the high aspect ratio cylindrical implant, according to an exemplary embodiment. High aspect ratio cylindrical (e.g., having a length to cross-sectional width ratio of greater than 10) implants may be used during surgical procedures such as for burr holes and osteoporotic/revision haloed pedicles as well as by cardiothoracic surgeons to press onto each sternal edge before closing with wires. In some embodiments, the high aspect ratio cylindrical implants have a first dimension (e.g., cross-sectional width or diameter) in a range of about 2 mm to about 4 mm, inclusive, and a second dimension (e.g., length) in a range of about 4 mm to about 8 mm, inclusive. In some embodiments, the string shaped implants have a first dimension (e.g., cross-sectional width or diameter) in the range of about 4.5 mm to about 8.5 mm, inclusive, and a second dimension (e.g., length) in a range of about 20 mm to about 180 mm, inclusive. In some embodiments, the second dimension is in the range of about 20 mm to about 90 mm, inclusive (e.g., for use in pediatric patients).

FIG. 9A depicts the extruder 900 including a lever 902 rotatably coupled to a base 904 at a coupling point 906. Unmolded composition (e.g., a settable composition) is disposed within the base 904 and when the lever 902 is pressed, molded composition 930 is extruded out of an aperture 908, the molded composition having a high aspect ratio cylindrical shape. In some embodiments, a shape or size of the aperture 908 may be changed to allow for different shape and/or different thickness implants to be molded by the extruder 900. In some embodiments, the string shaped molded composition may be cut along its length after being molded to adjust a length of the molded composition. The extruder 900 may be a mechanical device that requires the lever to be pumped repeatedly to extrude the molded composition 930 or may be an electronic device that automatically extrudes the molded composition 930 at a predetermined rate and/or for a predetermined amount of time. The extruded molded composition 930 may be removed from the extruder 900 via a tool (e.g., knife, scalpel, etc.). FIG. 9B depicts a cut and prepared molded composition 930. The molded composition 930 is prepared for implantation during the surgical procedure and/or additional processing by a medical professional. In some embodiments, the molded composition 930 may be just one of several molded putties needed to form the final implant shape.

FIGS. 10A-10C depict the process of forming a half-moon shaped implant using a mold 1000, according to an exemplary embodiment. Half-moon shaped implants may be desired for surgical procedures such as lateral or medial wall rebuilding of acetabulums or glenoids. In some embodiments, the half-moon shaped implants may have a first dimension in the range of about 9 mm to about 13 mm, inclusive, with a mean of about 10 mm, a second dimension in the range of about 11 mm to about 19 mm, inclusive, with a mean of about 15 mm, and a width in the range of about 15 mm to about 25 mm, inclusive, with a mean of about 20 mm.

FIG. 10A depicts a perspective view of the mold 1000 including a top portion 1002 and a bottom portion 1004. In some embodiments, the top portion 1002 and the bottom portion 1004 are coupled (e.g., via a fastener, hinge, etc.) that allows for the mold 1000 to be opened, to allow for unmolded composition (e.g., a settable composition) to be disposed within, and closed, to allow for molding of the composition. The bottom portion 1004 includes a flat surface 1006 and two mold cavities 1008, each having a half-moon shape formed in the body of the bottom portion 1004. Including two mold cavities 1008 allows for two molded putties (e.g., implant halves, separate implants, etc.) to be formed simultaneously. While shown as including two mold cavities 1008, in other embodiments, the mold 1000 may include more than two mold cavities (e.g., 3, 4, 5, 6, or even more). The shape of the mold cavities 1008 directly corresponds to the desired shape and dimensions of the desired molded composition. In some embodiments, the top portion 1002 also includes at last one mold cavity. In some embodiments, the mold cavities 1008 and an at least one mold cavity located in the top portion 1002 are aligned to mold composition in conjunction. In some embodiments, the top portion 1002 only includes a flat surface configured to press the settable composition into the mold cavity of the bottom portion 1004. In some embodiments, the mold cavities 1008 may include features that allow for the molded composition to include attributes such as flash, guide tabs, or the like, when molded.

FIG. 10B depicts a perspective view of an unprocessed composition 1010 being removed from the cavity 1008 of the mold 1000. In some embodiments, the unprocessed composition 1010 may include additional pieces. The unprocessed composition 1010 was formed in the mold 1000 of FIG. 10A and additional unprocessed putties may be molded simultaneous in the two mold cavities 1008. The unprocessed composition 1012 includes flash 1012 and two guide tabs 1014. Both the flash 1012 and the guide tabs 1014 are remnants of the molding process. In some embodiments, the unprocessed composition 1010 does not include one or both of the flash 1012 and the guide tabs 1014. In some embodiments, the guide tabs 1014 may be utilized to remove the unprocessed composition 1010 from the mold cavity 1008.

FIG. 10C depicts a side view of a molded composition 1030 formed using the mold 1000 (e.g., after processing the unprocessed composition 1010). The molded composition 1030 has a half moon shape and is prepared for implantation during the surgical procedure and/or additional processing by a medical professional. In some embodiments, the molded composition 1030 may be just one of several molded putties needed to form the final implant shape.

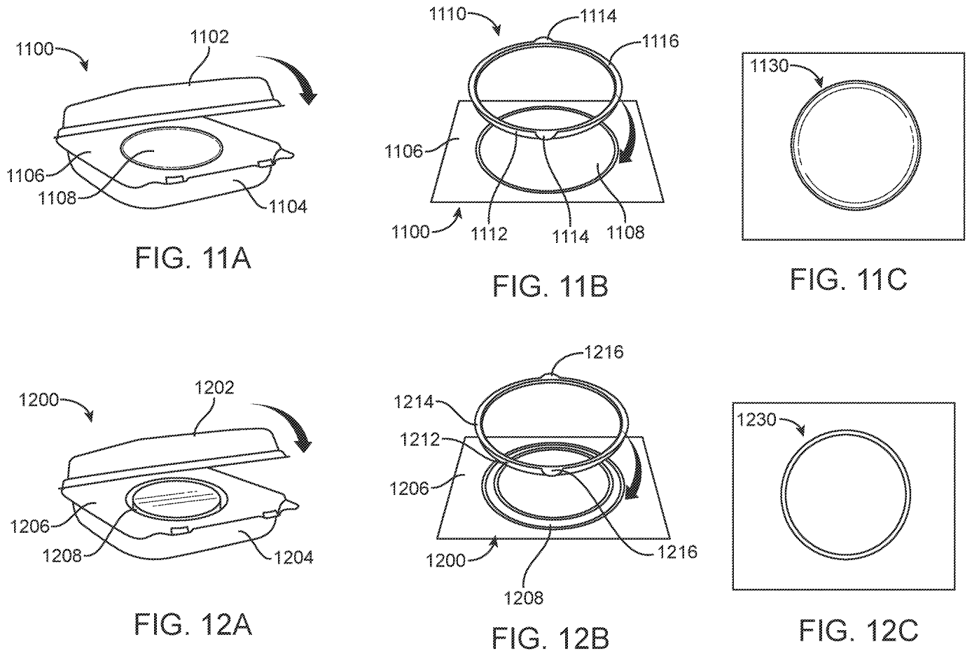

FIGS. 11A-11C depict the process of forming a circular implant using a mold 1100, according to an exemplary embodiment. Circular implants may be desired for surgical procedures supraorbital procedures and other anatomical voids. In some embodiments, circular implants have a first dimension (e.g., diameter) in a range of about 10 mm to about 25 mm, inclusive (e.g., about 10 mm, 12 mm, 14 mm, 16 mm, 20 mm, 22 mm, 24 mm, or 25 mm, inclusive).

FIG. 11A depicts a perspective view of the mold 1100 including a top portion 1102 and a bottom portion 1104. In some embodiments, the top portion 1102 and the bottom portion 1104 are coupled (e.g., via a fastener, hinge, etc.) that allows for the mold 1100 to be opened, to allow for unmolded composition (e.g., a settable composition) to be disposed within, and closed, to allow for molding of the composition. The bottom portion 1104 includes a flat surface 1106 and a mold cavity 1108 having a circular shape formed in the bottom portion 1104. The shape of the mold cavity 1108 directly corresponds to the desired shape and dimensions of the desired molded composition. In some embodiments, the top portion 1102 also includes at last one mold cavity. In some embodiments, the mold cavity 1108 and an at least one mold cavity located in the top portion 1102 are aligned to mold composition in conjunction. In some embodiments, the top portion 1102 only includes a flat surface configured to press the settable composition into the mold cavity of the bottom portion 1104. In some embodiments, the mold cavity 1108 may include features that allow for the molded composition to include attributes such as flash, guide tabs, or the like, when molded.

FIG. 11B depicts a perspective view of an unprocessed composition 610 being removed from the cavity 1108 of the mold 1100. In some embodiments, the unprocessed composition 1110 may include additional pieces. The unprocessed composition 1110 was formed in the mold 1100 of FIG. 11A. The unprocessed composition 1112 includes flash 1112 and two guide tabs 1114. Both the flash 1112 and the guide tabs 1114 are remnants of the molding process. In some embodiments, the unprocessed composition 1110 does not include one or both of the flash 1112 and the guide tabs 1114. In some embodiments, the guide tabs 1114 may be utilized to remove the unprocessed composition 1110 from the cavity 1108.

FIG. 11C depicts a side view of a molded composition 1130 molded using the mold 1100 (e.g., after forming and processing the unprocessed composition 1110). The molded composition 1130 has a circular shape and is prepared for implantation during the surgical procedure and/or additional processing by a medical professional. In some embodiments, the molded composition 1130 may be just one of several molded putties needed to form the final implant shape.

FIGS. 12A-12C depict the process of forming a ring-shaped implant using a mold 1200, according to an exemplary embodiment. Ring implants may be desired for surgical procedures such as for placement of native skull flaps back onto skull. In some embodiments, the ring implants have a circumference in the range of about 3 mm to about 7 mm, inclusive.

FIG. 12A depicts a perspective view of the mold 1200 including a top portion 1202 and a bottom portion 1204. In some embodiments, the top portion 1202 and the bottom portion 1204 are coupled (e.g., via a fastener, hinge, etc.) that allows for the mold 1200 to be opened, to allow for unmolded composition to be disposed within, and closed, to allow for molding of the composition. The bottom portion 1204 includes a flat surface 1206 and a mold cavity 1208 having a ring shape formed in the body of the bottom portion 1204. The shape of the mold cavity 1208 directly corresponds to the desired shape and dimensions of the desired molded composition. In some embodiments, the top portion 1202 also includes at last one mold cavity. In some embodiments, the mold cavity 1208 and an at least one mold cavity located in the top portion 1202 are aligned to mold composition in conjunction. In some embodiments, the top portion 1202 only includes a flat surface configured to press the settable composition into the mold cavity of the bottom portion 1204. In some embodiments, the mold cavity 1208 may include features that allow for the molded composition to include attributes such as flash, guide tabs, or the like, when molded.

FIG. 12B depicts a perspective view of an unprocessed composition 1210 being removed from the cavity 1208 of the mold 1200. In some embodiments, the unprocessed composition 1210 may include additional pieces. The unprocessed composition 1210 was formed in the mold 1200 of FIG. 12A. The unprocessed composition 1212 includes flash 1212 and two guide tabs 1214. Both the flash 1212 and the guide tabs 1214 are remnants of the molding process. In some embodiments, the unprocessed composition 1210 does not include one or both of the flash 1212 and the guide tabs 1214. In some embodiments, the guide tabs 1214 may be utilized to remove the unprocessed composition 1210 from the cavity 1208.

FIG. 12C depicts a side view of a molded composition 1230 formed using the mold 1200 (e.g., after processing the unprocessed composition 1210). The molded putty 1230 has a ring shape and is prepared for implantation during the surgical procedure and/or additional processing by a medical professional. In some embodiments, the molded composition 1230 may be just one of several molded putties needed to form the final implant shape.

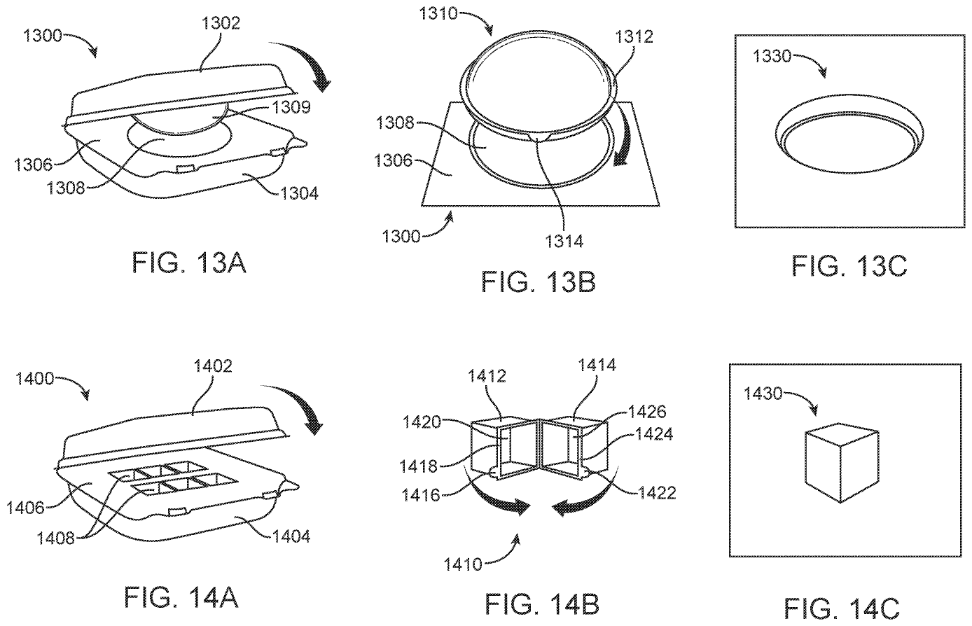

FIGS. 13A-13C depict the process of forming a dome shaped implant using a mold 1300, according to an exemplary embodiment. Dome shaped implants may be desired for surgical procedures involves cranial flaps, patellas and/or talus. In some embodiments, such as those when dome implants are used for patellas, the dome implant may have a first dimension (e.g., diameter) in a range of about 18 mm to about 26 mm, inclusive, with a mean of about 20 mm, a height in a range of about 22 mm to about 38 mm, inclusive, with a mean of about 31 mm, and a width in the range of about 31 mm to about 51 mm with a mean of about 40 mm.

FIG. 13A depicts a perspective view of the mold 1300 including a top portion 1302 and a bottom portion 1304. In some embodiments, the top portion 1302 and the bottom portion 1304 are coupled (e.g., via a fastener, hinge, etc.) that allows for the mold 1300 to be opened, to allow for unmolded composition (e.g., a settable composition) to be disposed within, and closed, to allow for molding of the composition. The bottom portion 1304 includes a flat surface 1306 and a mold cavity 1308 extending away from the flat surface 1308 and into the body of the bottom portion 1304. The shape of the mold cavities 1308 directly corresponds to the desired shape and dimensions of the desired molded composition. The top portion includes a dome shaped protrusion 1309 that extends into the mold cavity 1308 when the mold 1300 is closed. The protrusion 1309 impresses upon the unmolded composition as the protrusion 1309 extends into the mold cavity 1308 when the top portion 1302 and the bottom portion 1304 are brought close to each other causing the implant to be formed into a dome shape instead of a hemisphere. In some embodiments, the mold cavities 1308 may include features that allow for the molded composition to include attributes such as flash, guide tabs, or the like, when molded.

FIG. 13B depicts a perspective view of an unprocessed composition 1310 being removed from the cavity 1308 of the mold 1300. In some embodiments, the unprocessed composition 1310 may include additional pieces. The unprocessed composition 1310 was formed in the mold 1300 of FIG. 13A. The unprocessed composition 1312 includes flash 1312 and a guide tab 1314. Both the flash 1312 and the guide tab 1314 are remnants of the molding process. In some embodiments, the unprocessed composition 1310 does not include one or both of the flash 1312 and the guide tabs 1314. In some embodiments, the guide tabs 1314 may be utilized to remove the unprocessed composition 1310 from the mold cavity 1308.

FIG. 13C depicts a perspective view of a molded composition 1330 formed using the mold 1300 (e.g., after forming and processing the unprocessed composition 1310). The molded composition 1330 has a dome shape and is prepared for implantation during the surgical procedure and/or additional processing by a medical professional. In some embodiments, the molded composition 1330 may be just one of several molded putties needed to form the final implant shape.

FIGS. 14A-14C depict the process of forming a square shaped implant using a mold 1300, according to an exemplary embodiment. Square implants may be desired for surgical procedures such as sagittal split osteotomies. In some embodiments, a first dimension of the implant may be in the range of about 2 mm to about 4 mm, inclusive, a second dimension may be in the range of about 2 mm to about 6 mm, and a third dimension may be in the range of about 2 mm to about 6 mm, inclusive.

FIG. 14A depicts a perspective view of the mold 1400 including a top portion 1402 and a bottom portion 1404. In some embodiments, the top portion 1402 and the bottom portion 1404 are coupled (e.g., via a fastener, hinge, etc.) that allows for the mold 1400 to be opened, to allow for unmolded composition (e.g., a settable composition) to be disposed within, and closed, to allow for molding of the composition. The bottom portion 1404 includes a flat surface 1406 and a plurality of mold cavities 1408, each having a square or cube shape extending away from the flat surface 1408 and into the body of the bottom portion 1404. Including multiple mold cavities 1408 allows for multiple molded putties (e.g., implant halves, separate implants, etc.) to be formed simultaneously. The shape of the mold cavities 1408 directly corresponds to the desired shape and dimensions of the desired molded composition. In some embodiments, the top portion 1402 also includes at least one mold cavity. In some embodiments, the mold cavity 1408 and the at least one mold cavity located in the top portion 1402 are aligned to mold composition in conjunction with each other. In some embodiments, the top portion 1402 only includes a flat surface configured to press the settable composition into the mold cavities 1408 of the bottom portion 1404. In some embodiments, the mold cavities 1408 may include features that allow for the molded composition to include attributes such as flash, guide tabs, or the like, when molded.

FIG. 14B depicts a perspective view of an unprocessed composition 1410 including a first composition half 1412 and a second composition half 1414. In some embodiments, the unprocessed composition 1410 may include additional pieces. Both the first composition half 1412 and the second composition half 1414 are formed by the mold 1400 of FIG. 14A and may be formed simultaneously in the two mold cavities 1408. In some embodiments, the first composition half 1412 and the second composition half 1414 are formed together during one molding operation or are formed during separate molding operations. The first composition half 1412 includes a guide tab 1416 and flash 1418. Both the guide tab 1416 and the flash 1418 are remnants of the molding process. In some embodiments, the first composition half 1412 may not include one or both of the guide tab 1416 and the flash 1418. The first composition half 1412 also includes a flat surface 1420 which serves as a coupling point between the first composition half 1412 and the second composition half 1414. In some embodiments, the flat surface 1420 may be prepared (e.g., scored, marked, etc.) prior to coupling the first composition half 1412 and the second composition half 1414. The second composition half 1414 includes a guide tab 1422 and flash 1424, which correspond to the guide tab 1416 and the flash 1418 and are substantially similar. In some embodiments, the second composition half 1414 may not include one or both of the guide tab 1422 and the flash 1424. The second composition half 1414 includes as flat surface 1426 which corresponds to and couples to the flat surface 1420. Once the first composition half 1412 and the second composition half 1414 are joined together at the flat surfaces (1420 and 1426), the flash (1418 and 1424) and the guide tabs (1416 and 1422) may be removed during processing to prepare the implant for implantation.

FIG. 14C depicts a side view of a molded composition 1430 formed using the mold 1400 (e.g., after forming and processing the unprocessed composition 1410). The molded composition 1430 has a square shape and is prepared for implantation during the surgical procedure and/or additional processing by a medical professional. In some embodiments, the molded composition 1430 may be just one of several molded putties needed to form the final implant shape.

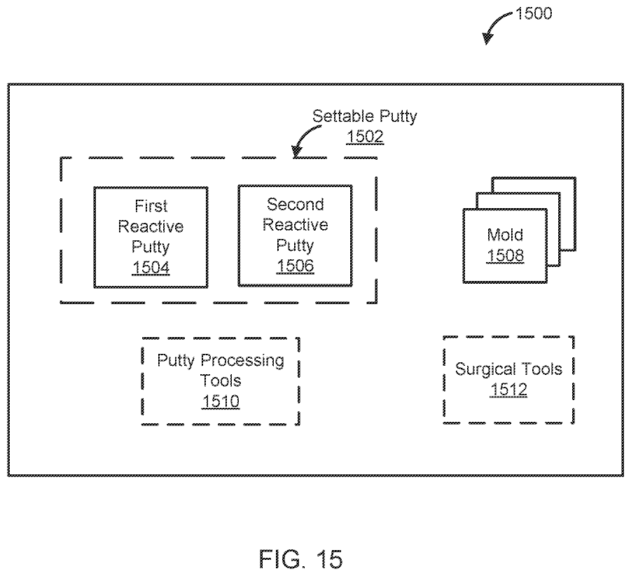

FIG. 15 is a block diagram for a kit 1500 for molding implants, according to an exemplary embodiment. The kit 1500 may include a settable composition 1502, which may include a first composition 1504 (e.g., a first reactive composition) and a second composition 1506 (e.g., a second reactive composition), at least one mold 1508, and optionally, processing tools 1510, and surgical tools 1512. The kit 1500 may be used in an operating room to aid in at least a portion of a surgical procedure by providing all the parts needed to form an implant in the operating room during a single procedure.

The settable composition 1502 is the formed and ready to mold composition that is used to create the implants and may be absorbable or non-absorbable (e.g., include any of the settable compositions described herein). The kit 1500 includes the settable putty 1502 as a first component 1504 and a second component 1506 that combine to create the settable composition 1502. Providing the settable composition 1502 as two separate, and non-setting components (e.g. reactive components) allows for the kit to be used when needed and for the settable composition to be formed during a surgical procedure. In other embodiments, the settable composition 1502 may include a one-part composition that may activated using an appropriate stimulus (e.g., heat, UV light, air, or any other suitable stimuli).

In some embodiments, the at least one mold 1508 may include a set of molds used to form the settable composition 1502 into implants having various implant shapes (e.g., one or more of the implant shapes described herein) for a variety of surgical procedures. Including a plurality of implants allows for a medical professional to choose the desired mold 1508 for the present surgical application.

The processing tools 1510 are a set of tools for processing the settable composition 1502 after it has been molded in a mold 1508. The tools may allow the medical professional to precisely smooth, cut, form, remove, add composition. Examples of processing tools 1510 that may be included in the kit 1500 include, but are not limited to scissors, cutters, spatulas, any other processing tool, or a combination thereof. In some embodiments, the kit 1500 may not include a set of processing tools 1510. The surgical tools 1512 may be additional tools used during the surgical procedure which may be configured to aid a medical professional in implanting the implant formed using the at least one mold 1508 included in the kit 1500 implant during a surgical procedure. Such surgical tools 1512 may include, but are not limited to knives, scalpels, forceps, clamps, any other surgical instrument, or a combination thereof.

### Absorbable Putty Compositions

Examples of absorbable putty compositions that can be used in the method 10 or with any of the molds described herein can be found in U.S. Pat. No. 11,116,866, issued Sep. 14, 2021, and entitled “Multi-Putty Adhesive and Cement Compositions for Tissue Hemostasis, Repair, and Construction”, U.S. Pat. No. 11,672,885, issued Jun. 13, 2023, entitled “Therapeutic Putties Containing Additives Including Processed Human Blood Plasma,” and US Patent Application Publication No. US 2022-0241457 A1, published on Aug. 4, 2022, entitled “Multi-Putty Bone Hemostatic And Adhesive Compositions For Use In Methods Of Installing And Securing Surgical Hardware In Bones,” all of which are incorporated by reference herein in their entireties.

Absorbable Putty Compositions include bone hemostatic and adhesive composition comprising a first component (also referred to as component A) and a second component (also referred to as component B), wherein the first component comprises a polyfunctional isocyanate compound, one or more polyols, and a particulate component, wherein the second putty comprises a polyfunctional isocyanate compound one or more polyols and a particulate component, wherein the particulate component comprises one or more particulate materials, and wherein the first and the second components are mixed together to form a bioabsorbable, homogenous putty.

In some embodiments, the first component comprises 30% to 40% of a polyfunctional isocyanate compound, 0% to 3% of one or more polyols, and 50%-65% of a particulate component, based upon the total weight of the first putty, the second putty comprises 3% to 5% of a polyfunctional isocyanate compound, 0.5% to 15% of one or more polyols, and 70% to 85% of a particulate component, based upon the total weight of the second putty, wherein the particulate component comprises one or more particulate materials, and wherein the first and the second putties are mixed together to form a bioabsorbable, homogenous putty.

In some embodiments, the first component comprises 30% to 35% of a polyfunctional isocyanate compound, based upon the total weight of the first putty, and the second component comprises 3% to 5% of a polyfunctional isocyanate compound, based upon the total weight of the second component. In some embodiments, the first component comprises 30% to 35% (i.e., 30% to 31%, 31% to 32%, 32% to 33%, 33% to 34% or 34% to 35%) of a polyfunctional isocyanate compound, based upon the total weight of the first component. In some embodiments, the second component comprises 3% to 5% (i.e., 3% to 3.5%, 3.5% to 4%, 4% to 4.5% or 4.5% to 5%) of a polyfunctional isocyanate compound, based upon the total weight of the second component.

In some embodiments, the first component comprises 35% to 40% of a polyfunctional isocyanate compound, based upon the total weight of the first component, and the second component comprises 3% to 5% of a polyfunctional isocyanate compound, based upon the total weight of the second component. In some embodiments, the first component comprises 35% to 40% (i.e., 35%-36%, 36% to 37%, 37% to 38%, 38% to 39% or 39% to 40%) of a polyfunctional isocyanate compound, based upon the total weight of the first component. In some embodiments, the second component comprises 3% to 5% (i.e., 3% to 3.5%, 3.5% to 4%, 4% to 4.5% or 4.5% to 5%) of a polyfunctional isocyanate compound, based upon the total weight of the second component.

In some embodiments, the first component comprises 2% to 3% of one or more polyols, based upon the total weight of the first putty, and the second putty comprises 0.5% to 5% of one or more polyols, based upon the total weight of the second putty. In some embodiments, the first component comprises 2% to 3% (i.e., 2% to 2.2%, 2.2% to 2.4%, 2.4% to 2.6%, 2.6% to 2.8% or 2.8% to 3%) of one or more polyols, based upon the total weight of the first component, and the second component comprises 0.5% to 5% (i.e., 0.5% to 0.6%, 0.6% to 0.7%, 0.7% to 0.8%, 0.8% to 0.9%, 0.9% to 1%, 1% to 2%, 2% to 3%, 3% to 4%, or 4% to 5%) of one or more polyols, based upon the total weight of the second component.

In some embodiments, the first component comprises 2% to 3% of one or more polyols, based upon the total weight of the first component, and the second component comprises 1.5% to 5% of one or more polyols, based upon the total weight of the second component. In some embodiments, the first component comprises 2% to 3% (i.e., 2% to 2.2%, 2.2% to 2.4%, 2.4% to 2.6%, 2.6% to 2.8% or 2.8% to 3%) of one or more polyols, based upon the total weight of the first component, and the second component comprises 1.5% to 5% (i.e., 1.5% to 1.6%, 1.6% to 1.7%, 1.7% to 1.8%, 1.8% to 1.9%, 1.9% to 2%, 2% to 2.1%, 2.1% to 2.2%, 2.2% to 2.3%, 2.3% to 2.4%, 2.4% to 2.5%, 2.5% to 2.6%, 2.6% to 2.7%, 2.7% to 2.8%, 2.8% to 2.9% or 2.9% to 3%) of one or more polyols, based upon the total weight of the second component.

In some embodiments, the first component comprises 0% to 2% of one or more polyols, based upon the total weight of the first component, and the second component comprises 2% to 10% of one or more polyols, based upon the total weight of the second component. In some embodiments, the first component comprises 0% to 2% (i.e., 0% to 0.1%, 0.1% to 0.2%, 0.2% to 0.3%, 0.3% to 0.4%, 0.4% to 0.5%, 0.5% to 0.6%, 0.7% to 0.8%, 0.8% to 0.9%, 0.9% to 1%, 1% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9% or 1.9% to 2%) of one or more polyols, based upon the total weight of the first component, and the second component comprises 2% to 10% (2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5% or 9.5% to 10%) of one or more polyols, based upon the total weight of the second component.

In some embodiments, the first component comprises 0% to 2% of one or more polyols, based upon the total weight of the first component, and wherein the second component comprises 10% to 15% of one or more polyols, based upon the total weight of the second component. In some embodiments, the first component comprises 0% to 2% (i.e., 0% to 0.1%, 0.1% to 0.2%, 0.2% to 0.3%, 0.3% to 0.4%, 0.4% to 0.5%, 0.5% to 0.6%, 0.7% to 0.8%, 0.8% to 0.9%, 0.9% to 1%, 1% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8% or 1.8% to 1.9% or 1.9% to 2%) of one or more polyols, based upon the total weight of the first component, and wherein the second component comprises 10% to 15% (10% to 10.5%, 10.5% to 11%, 11% to 11.5%, 11.5% to 12%, 12% to 12.5%, 12.5% to 13%, 13% to 13.5%, 13.5% to 14%, 14% to 14.5% or 14.5% to 15%) of one or more polyols, based upon the total weight of the second component.

In some embodiments, the first component comprises less than 0.5% of one or more polyols, based upon the total weight of the first putty, and the second putty comprises 0.5 to 15% of one or more polyols, based upon the total weight of the second putty. In a preferred embodiment, the first component comprises 0% of one or more polyols, based upon the total weight of the first putty.

In some embodiments, the first component comprises 30% to 40% of a polyfunctional isocyanate compound, less than 0.5% of one or more polyols, and 50-65% of a particulate component, based upon the total weight of the first putty, the second putty comprises 3% to 5% of a polyfunctional isocyanate compound, 0.5 to 15% of one or more polyols, and 70% to 85% of a particulate component, based upon the total weight of the second putty, wherein the particulate component comprises one or more particulate materials, and wherein the first and the second putties are mixed together to form a bioabsorbable, homogenous putty.

In some embodiments, the first component comprises less than 0.5% of one or more polyols, based upon the total weight of the first component, and the second component comprises 2% to 10% of one or more polyols, based upon the total weight of the second component. In some embodiments, the first component comprises less than 0.5% (i.e., 0% to 0.1%, 0.1% to 0.2%, 0.2% to 0.3%, 0.3% to 0.4% or 0.4% to 0.49%) of one or more polyols, based upon the total weight of the first component, and the second component comprises 2% to 10% (2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5% or 9.5% to 10%) of one or more polyols, based upon the total weight of the second component.

In some embodiments, the first component comprises less than 0.5% of one or more polyols, based upon the total weight of the first component, and wherein the second component comprises 10% to 15% of one or more polyols, based upon the total weight of the second component. In some embodiments, the first component comprises less than 0.5% (i.e., 0% to 0.1%, 0.1% to 0.2%, 0.2% to 0.3%, 0.3% to 0.4% or 0.4% to 0.49%) of one or more polyols, based upon the total weight of the first component, and wherein the second component comprises 10% to 15% (10% to 10.5%, 10.5% to 11%, 11% to 11.5%, 11.5% to 12%, 12% to 12.5%, 12.5% to 13%, 13% to 13.5%, 13.5% to 14%, 14% to 14.5% or 14.5% to 15%) of one or more polyols, based upon the total weight of the second component.

In some embodiments, the first component comprises 60% to 65% of a particulate component, based upon the total weight of the first component, and the second component comprises 80% to 85% of a particulate component, based upon the total weight of the second component. In some embodiments, the first component comprises 60% to 65% (60% to 61%, 61% to 62%, 62% to 63%, 63% to 64% or 64% to 65%) of a particulate component, based upon the total weight of the first component, and the second component comprises 80% to 85% (i.e., 80% to 81%, 81% to 82%, 82% to 83%, 83% to 84% or 84% to 85%) of a particulate component, based upon the total weight of the second component.

In some embodiments, the first component comprises 55% to 60% of a particulate component, based upon the total weight of the first component, and the second component comprises 80% to 85% of a particulate component, based upon the total weight of the second component. In some embodiments, the first component comprises 55% to 60% (i.e., 55% to 56%, 56% to 57%, 57% to 58%, 58% to 59%, or 59% to 60%) of a particulate component, based upon the total weight of the first component, and the second component comprises 80% to 85% (i.e., 80% to 81%, 81% to 82%, 82% to 83%, 83% to 84% or 84% to 85%) of a particulate component, based upon the total weight of the second component.

In some embodiments, the first component comprises 50% to 55% (50% to 51%, 51% to 52%, 52% to 53%, 53% to 54% or 54% to 55%) of a particulate component, based upon the total weight of the first component, and the second component comprises 70% to 75% (70% to 71%, 71% to 72%, 72% to 73%, 73% to 74% or 74% to 75%) of a particulate component, based upon the total weight of the second component.

In some embodiments, the homogenous component formed by mixing the first and the second components, comprises 50% to 75% of a particulate component, based upon the total weight of the homogenous component. In some embodiments, the homogenous component formed by mixing the first and the second components, comprises 50% to 74% (i.e., 50% to 52%, 52% to 54%, 54% to 56%, 56% to 58%, 57% to 60%, 60% to 62%, 62% to 64%, 64% to 66%, 66% to 68%, 68% to 70%, 70% to 72%, or 72% to 74%) of a particulate component, based upon the total weight of the homogenous component.

In some embodiments, the particulate matter is any one of calcium phosphate, siliconized calcium phosphate, substituted calcium phosphates, calcium pyrophosphate, calcium stearate, barium sulfate, a calcium, magnesium, zinc, aluminum or barium salt of lauric acid, a calcium, magnesium, zinc, aluminum or barium salt of myristic acid, a calcium, magnesium, zinc, aluminum or barium salt of palmitic acid, a calcium, magnesium, zinc, aluminum and/or barium salt of stearic acid, a calcium, magnesium, zinc, aluminum or barium salt of arachidic acid, a calcium, magnesium, zinc, aluminum or barium salt of behenic acid, polyethylene glycol (PEG) laurate, PEG stearate, PEG palmitate, PEG behenate hydroxyapatite, polymethyl methacrylate, glass-ionomer, absorbable phosphate glass, calcium sulfate, tricalcium phosphate, calcium carbonate, magnesium carbonate, aluminum carbonate, iron carbonate, zinc carbonate, calcium bicarbonate, sodium bicarbonate, demineralized bone, or mineralized bone. In a preferred embodiment, the particulate component is calcium phosphate, calcium stearate or a combination thereof.

In some embodiments, the particulate component of the first component comprises 0% to 1% of calcium stearate, and 53% to 62% of calcium phosphate, based upon the total weight of the first component, and the particulate component of the second component comprises 0.1% to 0.5% of calcium stearate, and 70% to 84% of calcium phosphate, based upon the total weight of the second component. In some embodiments, the particulate component of the first component comprises 0% to 1% (0% to 0.1%, 0.1% to 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.9% to 1%) of calcium stearate, and 53% to 62% (53% to 54%, 54% to 55%, 55% to 56%, 56% to 57%, 57% to 58%, 58% to 59%, 59% to 60%, 60% to 61% or 61% to 62%) of calcium phosphate, based upon the total weight of the first component, and the particulate component of the second component comprises 0.1% to 0.5% of calcium stearate (0.1% to 0.2%, 0.2% to 0.3%, 0.3% to 0.4% or 0.4% to 0.5%), and 70% to 84% (70% to 71%, 71% to 72%, 72% to 73%, 73% to 74%, 74% to 75%, 75% to 76%, 76% to 77%, 77% to 78%, 78% to 79%, 79% to 80%, 80% to 81%, 81% to 82%, 82% to 83%, 83% to 84%) of calcium phosphate, based upon the total weight of the second component.

In some embodiments, the homogenous composition formed by mixing the first and the second components, comprises 0.2% to 1% (0.2% to 0.3%, 0.3% to 0.4%, 0.4% to 0.5%, 0.5% to 0.6%, 0.6% to 0.7%, 0.7% to 0.8%, 0.8% to 0.9% or 0.9% to 1%) of calcium stearate, and 56% to 73% (56% to 57%, 57% to 58%, 58% to 59%, 59% to 60%, 60% to 61%, 61% to 62%, 62% to 63%, 63% to 64%, 64% to 65%, 65% to 66%, 66% to 67%, 67% to 68%, 68% to 69%, 69% to 70%, 70% to 71%, 71% to 72% or 72% to 73%) of calcium phosphate, based upon the total weight of the homogenous composition.

In some embodiments, the particulate material of the first component comprises barium sulfate, calcium stearate, calcium phosphate or a combination thereof. In some embodiments, the particulate material of the first component comprises 1% to 10% (i.e., 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5% or 9.5% to 10%) of barium sulfate, 1% to 2% (i.e., 1% to 1.25%, 1.25% to 1.5%, 1.5% to 1.75% or 1.75% to 2%) of calcium stearate and 48% to 53% (i.e., 48% to 49%, 49% to 50%, 50% to 51%, 51% to 52% or 52% to 53%) of calcium phosphate, based upon the total weight of the first component, and the second component comprises 0.1% to 0.5% (0.1% to 0.2%, 0.2% to 0.3%, 0.3% to 0.4% or 0.4% to 0.5%) of calcium stearate and 70% to 75% (i.e., 70% to 71%, 71% to 72%, 72% to 73%, 73% to 74% or 74% to 75%) of calcium phosphate, based upon the total weight of the second component.

In some embodiments, the homogenous composition formed by mixing the first and the second component, comprises 0.5% to 8.5% (i.e., 0.5% to 0.6%, 0.6% to 0.7%, 0.7% to 0.8%, 0.8% to 0.9%, 0.9% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8% or 8% to 8.5%) of barium sulfate, 0.2% to 1% of calcium stearate (i.e., 0.2% to 0.3%, 0.3% to 0.4% or 0.4% to 0.5%, 0.5% to 0.6%, 0.6% to 0.7%, 0.7% to 0.8%, 0.8% to 0.9% or 0.9% to 1%) and 50% to 65% of calcium phosphate (50% to 52%, 52% to 54%, 54% to 56%, 56% to 58%, 58% to 60%, 60% to 62% or 62% to 65%) based on the weight of the homogenous composition.

In some embodiments, the homogenous component formed by mixing the first and the second components, as described herein, comprises about 0.1% to about 5% of one or more additives based upon total weight of the homogenous component. In some embodiments, the homogenous component formed by mixing the first and the second components, comprises about 0.1% to about 5% (i.e., 0.1% to 0.2%, 0.2% to 0.3%, 0.3% to 0.4%, 0.4% to 0.5%, 0.5% to 0.6%, 0.6% to 0.7%, 0.7% to 0.8%, 0.8% to 0.9%, 0.9% to 1%, 1% to 1.5%, 1.5% to 2%, 2% to 2.5%, 2.5% to 3%, 3% to 3.5%, 3.5% to 4%, 4% to 4.5% or 4.5% to 5%) of one or more additives based upon total weight of the homogenous component.

In some embodiments, the homogenous component formed by mixing the first and the second components, comprises about 0.9% to about 5% of one or more additives, based upon the total weight of the homogenous component. In some embodiments, the homogenous component formed by mixing the first and the second components, comprises about 0.9% to about 5% (i.e., 0.9% to 1%, 1% to 1.5%, 1.5% to 2%, 2% to 2.5%, 2.5% to 3%, 3% to 3.5%, 3.5% to 4%, 4% to 4.5% or 4.5% to 5%) of one or more additives, based upon the total weight of the homogenous component.

In some embodiments, the homogenous component formed by mixing the first and the second components, comprises about 1% to about 5% of one or more additives, based upon the total weight of the homogenous component. In some embodiments, the homogenous component formed by mixing the first and the second components, comprises about 1% to about 5% (i.e., 1% to 1.5%, 1.5% to 2%, 2% to 2.5%, 2.5% to 3%, 3% to 3.5%, 3.5% to 4%, 4% to 4.5%, or 4.5% to 5%) of one or more additives, based upon the total weight of the homogenous component. As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

In some embodiments, the one or more additives is any one of an antioxidant, a colorant, a steroid, tocopheryl acetate, or triacetin or a combination thereof.

In some embodiments, the first component comprises 0% of triacetin, based upon the total weight of the first component, and the second component comprises about 0.5% to about 2% of triacetin, based upon the total weight of the second component. In some embodiments, the first component comprises 0% of triacetin, based upon the total weight of the first component, and the second component comprises about 0.5% to about 2% (0.5% to 0.6%, 0.6% to 0.7%, 0.7% to 0.8%, 0.8% to 0.9%, 0.9% to 1%, 1% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9% or 1.9% to 2%) of triacetin, based upon the total weight of the second component.

In some embodiments, the homogenous component formed by mixing the first and the second components, comprises about 0.1% to about 1% triacetin, based upon the total weight of the homogenous component. In some embodiments, the homogenous component formed by mixing the first and the second component, comprises about 0.1% to about 1% (0.1% to 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.9% to 1%) of triacetin, based upon the total weight of the homogenous component.

In some embodiments, the first component comprises about 6% to about 7% of tocopheryl acetate, based upon the total weight of the first component, and the second component comprises about 0% to about 4% of tocopheryl acetate, based upon the total weight of the second component. In some embodiments, the first component comprises about 6% to about 7% (6% to 6.1%, 6.1% to 6.2%, 6.2% to 6.3%, 6.3% to 6.4%, 6.4% to 6.5%, 6.5% to 6.6%, 6.6% to 6.7%, 6.7% to 6.8%, 6.8% to 6.9% or 6.9% to 7%) of tocopheryl acetate, based upon the total weight of the first component, and the second component comprises about 0% to about 4% (0% to 0.5%, 0.5% to 1%, 1.5% to 2%, 2% to 2.5%, 2.5% to 3%, 3% to 3.5%, 3.5% to 4%) of tocopheryl acetate, based upon the total weight of the second component.

In some embodiments, the homogenous component formed by mixing the first and the second components, comprise about 5% to about 6% of tocopheryl acetate, based upon the total weight of the homogenous putty. In some embodiments, the homogenous component formed by mixing the first and the second components, comprise about 5% to about 6% (5% to 5.1%, 5.1% to 5.2%, 5.2% to 5.3%, 5.3% to 5.4% or 5.4% to 5.5%, 5.5% to 5.6%, 5.6% to 5.7%, 5.7% to 5.8%, 5.8% to 5.9% or 5.9% to 6%) of tocopheryl acetate, based upon the total weight of the homogenous components.

In some embodiment, the polyfunctional isocyanate compound is an aromatic isocyanate, an aliphatic isocyanate, a cycloaliphatic isocyanate, and an adduct of an isocyanate. In some embodiments, the polyfunctional isocyanate compound is any one of an absorbable lactide diester (ALD), ethyl lysine diisocyanate, cyclohexyl diisocyanate, hexamethylene diisocyanate, a hexamethylene diisocyanate trimer, a hexamethylene diisocyanate biuret, a methylene bis-diphenyldiisocyanate, a lysine diisocyanate, a diphenylmethanediisocyanate (MDI), 4,4-diphenylmethanediisocyanate, a mixture of 2,4′-diphenylmethanediisocyanate and 4,4′-diphenylmethanediisocyanate isomers, a polycarbodiimide-modified diphenylmethane disocyanate, polymethylene polyphenylisocyanate. In a preferred embodiment, the polyfunctional isocyanate compound is an absorbable lactide diester (ALD).

In some embodiments, the one or more polyols is any one of ethylene glycol, diethylene glycol, propanediol, 1,2-propanediol (propylene glycol), 1,3-propanediol, 1,2-ethanediol (ethylene glycol), butanediol, 1,4-butanediol, 1,5-pentanediol, 1,3-cyclopentanediol, 1,6-hexanediol, 1,8-octanediol, polytetramethylene ether glycols, polyols derived from glycolide, lactide, trimethylenecarbonate, p-dioxanone, polycaprolactone polyol, polycaprolactone triol polyester polyol, polyadipate polyol, diol, poly(butane-adipate) diol, poly(ethylene/propylene-adipate) diol, poly(hexane/adipate/isophthalate diol)), poly(oxypropylene) glycol, poly(oxytetramethylene) glycol, and poly(oxyethylene) glycols, polycaprolactone co-glycolide, a polycaprolactone co-lactide or a triethanolamine (TEOA) or a combination thereof. In a preferred embodiment, the one or more polyols is a polycaprolactone triol, a butanediol or a triethanolamine (TEOA) or a combination thereof.

In some embodiments, the homogenous component formed by mixing the first and the second components, comprises 20% to 40% of a polyurethane copolymer, based upon the total weight of the homogenous component. In some embodiments, the homogenous component formed by mixing the first and the second components, comprises 20% to 40% (i.e., 20% to 22%, 22% to 24%, 24% to 26%, 26% to 28%, 28% to 30%, 30% to 32%, 32% to 34%, 34% to 36%, 36% to 38% or 38% to 40%) of a polyurethane copolymer, based upon the total weight of the homogenous component.

In some embodiments, the homogenous component formed by mixing the first and the second components, comprise a polyurethane co-polymer comprising of an Absorbable Lactide Diester (ALD)-Polycaprolactone Triol (PCL)-Butanediol (BDO)-Triethanolamine (TEOA) polymer (ALD-PCL-BDO-TEOA polymer). In some embodiments, the homogenous putty formed by mixing the first and the second compositions, comprise a polyurethane co-polymer comprising of an Absorbable Lactide Diester-Polycaprolactone Triol-Butanediol (ALD-PCL-BDO) polymer.

In some embodiments, the bone hemostatic and adhesive composition described herein comprises a first component and a second component, wherein the first component comprises 30% to 35% of a polyfunctional isocyanate compound, 0% to 2% of one or more polyols, and 60%-65% of a particulate component, based upon the total weight of the first component, wherein the second component comprises 3% to 5% of a polyfunctional isocyanate compound, 1 to 5% of one or more polyols, and 80% to 85% of a particulate component, based upon the total weight of the second component, wherein the particulate component comprises one or more particulate matters, and wherein the first and the second components are mixed together to form a bioabsorbable, and homogenous component.

In some embodiment, the bone hemostatic and adhesive composition described herein comprises a first component and a second component, wherein the first component comprises 30% to 35% of a polyfunctional isocyanate compound, 0% to 3% of one or more polyols, 55%-60% of a particulate component, based upon the total weight of the first component, wherein the second component comprises 3% to 5% of a polyfunctional isocyanate compound, 0.5% to 5% of one or more polyols, and 80% to 85% of a particulate component, based upon the total weight of the second component, wherein the particulate component comprises one or more particulate matters, and wherein the first and the second components are mixed together to form a bioabsorbable, and homogenous component.

In some embodiment, the bone hemostatic and adhesive composition described herein comprises a first putty and a second putty, wherein the first putty comprises 35% to 40% of a polyfunctional isocyanate compound, and 50%-55% of a particulate component, based upon the total weight of the first putty, wherein the second putty comprises 3% to 5% of a polyfunctional isocyanate compound, 2% to 12% of one or more polyols, and 70% to 75% of a particulate component, based upon the total weight of the second putty, wherein the particulate component comprises one or more particulate matters, and wherein the first and the second putties are mixed together to form a bioabsorbable, homogenous putty.

In some embodiments, the first component, the second component and the homogenous composition formed by mixing the first and the second components are a putty. In some embodiments, the homogenous composition formed by mixing the first and the second components is moldable or hand-settable.

In some embodiments, the first component, the second component and the homogenous composition formed by mixing the first and the second components are a paste. In some embodiments, the homogenous composition formed by mixing the first and the second components is a flowable paste.

In some embodiments, the homogenous composition is formed by mixing from 1 part to 5 parts (1 part to 1.5 parts, 1.5 parts to 2 parts, 2 parts to 2.5 parts, 2.5 parts to 3 parts, 3 parts to 3.5 parts, 3.5 parts to 4 parts, 4 parts to 4.5 parts or 4.5 parts to 5 parts) of the first component and 1 part of the second component.

In one embodiment, the individual components are sterile.

In one embodiment, an optional chain extender, such as a diol or diamine, or a crosslinker, such as a triol or triamine, is added to one of the component parts before combining, or to the homogenous settable compositions formed from the combining of the component parts, in an amount sufficient to increase the rate of the curing reaction.

In one embodiment, the component parts of a composition described here are in the form of a putty or paste and may be combined, for example, by hand-kneading, or by extrusion, for example through a syringe, or by otherwise combining or compounding into a single homogenous composition. In one embodiment, the component parts each comprise an additive, such as a colorant or dye, such that the additives impart a different color to each component. In one embodiment, the separate additives, each of a different color, form a third new color when the components have been mixed to homogeneity, such that the new color is indicates that a single homogenous composition has been formed. In one embodiment, the component parts consist of part A and part B, part A comprises a colorant or dye which gives part A a red color, part B comprises a colorant or dye which gives part B a blue color, and a composition of a substantially purple color is formed from the combination of parts A and B to homogeneity. In one embodiment, the component parts can be mixed to homogeneity within about 1 minute or less, or within about 2 minutes.

In one embodiment, each component of a settable compositions described herein is in the form of a putty and the homogenous settable composition that results from their combination is also in the form of a putty for a period of time after initiation of the curing reaction. The term “putty” refers to a composition that is soft, moldable, preferably non-elastic, and cohesive.

In some embodiments, the first component, the second component and the homogenous composition formed by mixing the first and the second components are a putty. In some embodiments, the homogenous composition formed by mixing the first and the second components is moldable or hand-settable.

In some embodiments, the first component, the second component and the homogenous composition formed by mixing the first and the second components are a paste. In some embodiments, the homogenous composition formed by mixing the first and the second components is a flowable paste.

In some embodiments, the homogenous composition is formed by mixing from 1 part to 5 parts (1 part to 1.5 parts, 1.5 parts to 2 parts, 2 parts to 2.5 parts, 2.5 parts to 3 parts, 3 parts to 3.5 parts, 3.5 parts to 4 parts, 4 parts to 4.5 parts or 4.5 parts to 5 parts) of the first component and 1 part of the second component.

In one embodiment, the individual components are sterile.

In one embodiment, an optional chain extender, such as a diol or diamine, or a crosslinker, such as a triol or triamine, is added to one of the component parts before combining, or to the homogenous settable compositions formed from the combining of the component parts, in an amount sufficient to increase the rate of the curing reaction.

In one embodiment, the component parts of a composition described here are in the form of a putty or paste and may be combined, for example, by hand-kneading, or by extrusion, for example through a syringe, or by otherwise combining or compounding into a single homogenous composition. In one embodiment, the component parts each comprise an additive, such as a colorant or dye, such that the additives impart a different color to each component.

In one embodiment, the separate additives, each of a different color, form a third new color when the components have been mixed to homogeneity, such that the new color is indicates that a single homogenous composition has been formed. In one embodiment, the component parts consist of part A and part B, part A comprises a colorant or dye which gives part A a red color, part B comprises a colorant or dye which gives part B a blue color, and a composition of a substantially purple color is formed from the combination of parts A and B to homogeneity. In one embodiment, the component parts can be mixed to homogeneity within about 1 minute or less, or within about 2 minutes.

In one embodiment, each component of a settable compositions described herein is in the form of a putty and the homogenous settable composition that results from their combination is also in the form of a putty for a period of time after initiation of the curing reaction. The term “putty” refers to a composition that is soft, moldable, preferably non-elastic, and cohesive.

In one embodiment, a putty is formed as a suspension or dispersion of particulates within a liquid.

In one embodiment, the particulate component for inclusion in a component part of the compositions described here is selected from one or more of calcium sulfate, sodium phosphate, calcium aluminate, strontium phosphate, calcium strontium phosphate, tricalcium phosphate, calcium pyrophosphate, magnesium phosphate, hydroxyapatite, biomimetic carbonate apatite, biphasic calcium phosphate/hydroxyapatite, mineralized bone matrix, demineralized bone matrix, glass ionomer, absorbable phosphate glass and nonresorbable particulate metallic or polymeric materials such as stainless steel or titanium powder or nanoparticles, polyurethane, polyureaurethane, polymethacrylic acid and polyarylether ketones such as PEK, PEEK, PEKK, PEEKK and PEKEKK particles. Further examples of particulate materials are provided infra.

The particles of a particulate material used in the component parts of the compositions described here may be porous or non-porous particles. In one embodiment, the particles are porous and the degree of porosity is sufficient to permit the ingress of cells or fluids into the composition after its placement in situ. Particle size may also be varied from about 0.05 to less than or equal to 1 millimeter or 2 millimeters in diameter to control the consistency, with smaller particle sizes yielding smoother more cohesive putties.

The settable compositions described herein are biocompatible and suitable for use in vivo, particularly during surgery. The term “biocompatible” refers to materials that do not induce undesirable effects when administered or implanted in vivo, for example, an immune reaction and/or an inflammatory reaction, or other adverse reaction that is detrimental to wound healing and/or to the implant recipient. A biocompatible material may also be referred to as “nontoxic”. In one aspect, the biocompatible compositions described here form from a low-exotherm reaction and their formation does not produce toxic fumes or tissue-damaging amounts of heat. In another aspect, where the compositions are biodegradable, their degradation under physiological conditions does not produce toxic by-products and/or is not toxic to the implant recipient. In one embodiment, the maximum exotherm (amount of heat i.e., temperature increase, generated by the reaction) of the polymerization reaction is 20° C. or less, most preferably 10° C. or less.

In one embodiment, the compositions are fully or partially biodegradable. The terms “degradable”, “biodegradable”, “resorbable”, and “absorbable” and the like are used interchangeably herein to refer to the ability of the claimed compositions to degrade (partially or completely) under physiological conditions into non-toxic products that can be metabolized or excreted from the body within a period of time, generally several days and up to a year or about 18 to 24 months (i.e., 18, 19, 20, 21, 22, 23, or 24 months) or longer. In one embodiment, the composition is fully biodegradable within about 12 months. Compositions may be considered non-biodegradable if they remain stable in vivo for periods exceeding about ten years.

In one embodiment, the settable composition consists of at least two component parts, A and B, that when combined form a composition that cures into a fully hardened polymeric composition, the polymer selected from a polyurethane, a polyureaurethane, a polyetherurethane, or a polyesterurethane, over a period of time at body temperature (i.e., about 37 C). At least one of the component parts comprises an isocyanate component and one or both component parts comprise a polyol/polyamine component (for example, the first putty component may comprise a polyfunctional isocyanate, tocopherol acetate, calcium phosphate particles and a polyol and the second putty component may comprise a polyfunctional isocyanate, calcium phosphate particles, at least one polyol, a fatty acid salt and tocopherol acetate). The isocyanate component consists of an isocyanate monomer, polymer, prepolymer, or combination thereof. The isocyanate component may thus comprise one or more different isocyanates, as well as an isocyanate in both its monomeric form and its polymer or prepolymer form. The term “isocyanate” is used generically to refer to isocyanates, diisocyanates, and polyisocyanates. The term “polyol” in the context of the “polyol/polyamine component” refers to both diols and polyols. Thus, the polyol or polyamine component may comprise or consist of one or more different diols, polyols, polyamines, or mixtures of two or more diols, polyols and/or polyamines.

In one embodiment, the composition further comprises an additive selected from one or more of tocopherol esters (e.g., tocopheryl (Vitamin E) acetate (TA)), triglycerides, acetyl triethyl citrate, and fatty acid esters, to aid in handling properties and packaging. In one embodiment, the composition further comprises one or more additives selected from an antioxidant, an anhydrous particulate material, a colorant, a therapeutic agent, and a radiopaque agent. In one embodiment, the therapeutic agent is selected from one or more of an anticancer agent, an antimicrobial agent, an anesthetic agent, an analgesic agent, an anti-inflammatory agent, and an osteogenic agent.

In one embodiment, one or more of the components of the composition comprises a prepolymer. A prepolymer is a polymer having reactive end groups, e.g., isocyanate or hydroxyl groups. In one embodiment, the prepolymer comprises an excess of the isocyanate component relative to the polyol/polyamine component. In one embodiment of a two-component composition, one component comprises a prepolymer and no, or substantially no, unreacted polyol; and the second comprises or consists of a hydroxyl terminated prepolymer lacking free isocyanate groups and unreacted polyol or polyamine.

A low molecular weight polymer refers to a polymer having a number average molecular weight in the range of about 500 to 20,000 (i.e., 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or 500-1,000, 500-5,000, 500-10,000, 500-15,000, 1,000-5,000, 1,000-10,000, 1,000-15,000, 1,000-2,0000, 5,000-10,000, 5,000-15,000, 10,000-15,000, 10,000-20,000, or 15,000-20,000) or 500 to 10,000 ((i.e., 500, 1000, 1500, 2000, 2500, 3000, 3500, 4000, 4500, 5000, 5500, 6000, 6500, 7000, 7500, 8000, 8500, 9000, 9500, or 10,000 or 500-1,000, 500-5,000, 1,000-5,000, 1,000-10,000, or 5,000-10000). A prepolymer containing reactive isocyanate end groups is formed, for example, from the initial reaction of an excess of isocyanate with a limiting amount of polyol or polyamine.

Each of the components may also independently comprise an optional particulate material and an optional chain extender, crosslinker, or curative. For example, the first putty, the second putty, or both the first putty and the second putty may comprise one or more particulate materials selected from calcium sulfate, calcium phosphosilicate, sodium phosphate, calcium aluminate, or calcium phosphate. The first putty, the second putty, or both the first putty and the second putty may comprise one or more particulate materials selected from a polyaryletherketone-based material, a polymethylmethacrylate-based material, or a tantalum- or titanium-based filler. The first putty, the second putty, or both the first putty and the second putty may comprise one or more particulate materials selected from calcium sulfate, sodium phosphate, calcium aluminate, strontium phosphate, calcium strontium phosphate, tricalcium phosphate, calcium pyrophosphate, or magnesium phosphate. The first putty, the second putty, or both the first putty and the second putty may comprise one or more particulate materials selected from absorbable phosphate glass, nonresorbable particulate metallic materials (e.g. stainless steel powder, titanium powder, stainless steel nanoparticles, or titanium nanoparticles), or nonresorbable polymeric materials (e.g. polyurethane particles, polyureaurethane particles, polymethacrylic acid particles, and polyarylether ketone particles). The second putty may comprise one or more particulate materials selected from hydroxyapatite, biomimetic carbonate apatite, biphasic calcium phosphate/hydroxyapatite, mineralized bone matrix, demineralized bone matrix, or glass ionomer.

As discussed above, the components of the settable composition are provided as individual units, each containing reagents in amounts such that when the components are mixed together, they form a mixture that fully reacts or cures into a hardened composition after a period of time at room or body temperature. For example, where the settable composition comprises two putties, A and B, putty A comprises an excess of the isocyanate component relative to the polyol component and putty B comprises less of the isocyanate component and more of the polyol/polyamine component than putty A. Putty B also optionally comprises a chain extender and/or crosslinker. Each putty optionally contains an amount of particulate material suspended in the liquid components to form a composition having a putty-like consistency.

In one embodiment, the particulate material is selected from one or more of a polyurethane, calcium sulfate, calcium phosphosilicate, sodium phosphate, calcium aluminate, calcium phosphate, hydroxyapatite, demineralized bone, or mineralized bone. Other particulate materials may also be used, as described infra.

In one embodiment, putty A comprises 15-50% (i.e., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% or 15-20, 15-25, 15-30, 15-35, 15-40, 15-45, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50%) of the isocyanate component, 0.5-5% (i.e., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% or 0.5-1, 0.5-1.5, 0.5-2, 0.5-2.5, 0.5-3.0, 0.5-3.5, 0.5-4.0, 0.5-4.5, 1-1.5, 1-2, 1-2.5, 1-3, 1-3.5, 1-4, 1-4.5, 1-5, 1.5-2, 1.5-2.5, 1.5-3, 1.5-3.5, 1.5-4, 1.5-4.5, 1.5-5, 2-2.5, 2-3, 2-3.5, 2-4, 2-4.5, 2-5, 2.5-3, 2.5-3.5, 2.5-4, 2.5-4.5, 2.5-5, 3-3.5, 3-4, 3-4.5, 3-5, 3.5-4, 3.5-4.5, 3.5-5, 4-4.5, 4-5, or 4.5-5%) of the polyol component, and 40-85% (i.e., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85% or 40-45, 40-50, 40-55, 40-60, 40-65, 40-70, 40-75, 40-80, 45-50, 45-55, 45-60, 45-65, 45-70, 45-75, 45-80, 45-85, 50-55, 50-60, 50-65, 50-70, 50-75, 50-80, 50-85, 55-60, 55-65, 55-70, 55-75, 55-80, 55-85, 60-65, 60-70, 60-75, 60-80, 60-85, 65-70, 65-75, 65-70, 65-75, 65-80, 65-85, 70-75, 70-80, 70-85, 75-80, 75-85, or 80-85%) of the polyol component, and 40-75% (i.e., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70% or 40-45, 40-50, 40-55, 40-60, 40-65, 40-70, 45-50, 45-55, 45-60, 45-65, 45-70, 45-75, 50-55, 50-60, 50-65, 50-70, 50-75, 55-60, 55-65, 55-70, 55-75, 60-65, 60-70, 60-75, 65-70, 65-75, or 70-75%) particulate material, based upon total weight of putty A; putty B comprises 1-10% (i.e., 1, 2, 3, 4, 5, 6, 7, 8, 9, or 10% (i.e., 1, 2, 3, 4, 5, 6, 7, 8, 9, or 10% or 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10 or 9-10) of the isocyanate component, 3-15% (i.e., 3, 4, 5, 6, 7, 8, 9, 10, 11, 12, 13, 14, or 15% (i.e., 3, 4, 5, 6, 7, 8, 9, 10, 11, 12, 13, 14, or 15% or 3-5, 3-10, 5-10, 5-15, or 10-15)) of the polyol component, and 65-85% (i.e., 65, 66, 67, 68, 69, 70, 71, 72, 73, 74, 75, 76, 77, 78, 79, 80, 81, 82, 83, 84, or 85% or 65-70, 65-75, 65-80, 70-75, 70-80, 70-85, 75-80, 75-85, or 80-85%) particulate material, based upon total weight of putty B.

In one embodiment, putty A comprises 20-35% (i.e., 20, 21, 22, 23, 24, 25, 26, 27, 28, 29, 30, 31, 32, 33, 34, or 35% or 20-25, 20-30, 25-30, 25-35, or 30-35%) of the isocyanate component, 0.5-5% (i.e., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% or 0.5-1, 0.5-1.5, 0.5-2, 0.5-2.5, 0.5-3.0, 0.5-3.5, 0.5-4.0, 0.5-4.5, 1-1.5, 1-2, 1-2.5, 1-3, 1-3.5, 1-4, 1-4.5, 1-5, 1.5-2, 1.5-2.5, 1.5-3, 1.5-3.5, 1.5-4, 1.5-4.5, 1.5-5, 2-2.5, 2-3, 2-3.5, 2-4, 2-4.5, 2-5, 2.5-3, 2.5-3.5, 2.5-4, 2.5-4.5, 2.5-5, 3-3.5, 3-4, 3-4.5, 3-5, 3.5-4, 3.5-4.5, 3.5-5, 4-4.5, 4-5, or 4.5-5%) of the polyol component, and 40-85% (i.e., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85% or 40-45, 40-50, 40-55, 40-60, 40-65, 40-70, 40-75, 40-80, 45-50, 45-55, 45-60, 45-65, 45-70, 45-75, 45-80, 45-85, 50-55, 50-60, 50-65, 50-70, 50-75, 50-80, 50-85, 55-60, 55-65, 55-70, 55-75,55-80, 55-85, 60-65, 60-70, 60-75, 60-80, 60-85, 65-70, 65-75, 65-70, 65-75, 65-80, 65-85, 70-75, 70-80, 70-85, 75-80, 75-85, or 80-85%)) of the polyol component, and 50-75% particulate material, based upon total weight of putty A; putty B comprises 1-10% (i.e., 1, 2, 3, 4, 5, 6, 7, 8, 9, or 10% (i.e., 1, 2, 3, 4, 5, 6, 7, 8, 9, or 10% or 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10 or 9-10) of the isocyanate component, 3-15% (i.e., 3, 4, 5, 6, 7, 8, 9, 10, 11, 12, 13, 14, or 15% i.e., 3, 4, 5, 6, 7, 8, 9, 10, 11, 12, 13, 14, or 15% or 3-5, 3-10, 5-10, 5-15, or 10-15%) of the polyol component, and 75-85% (i.e., 75, 76, 77, 78, 79, 80, 81, 82, 83, 84, or 85% or 75-80 or 80-85%) particulate material, based upon total weight of putty B.

### The Isocyanate Component

In one embodiment, the isocyanate component comprises or consists of an aromatic isocyanate, an aliphatic isocyanate, a cycloaliphatic isocyanate, or an adduct of an isocyanate, or a mixture of any of the foregoing. A mixture refers to a mixture of two or more of the foregoing. For example, the isocyanate component may comprise or consist of a mixture of two or more isocyanates independently selected from an aromatic isocyanate, an aliphatic isocyanate, a cycloaliphatic isocyanate, and an adduct of an isocyanate.

In one embodiment, the isocyanate is an aliphatic isocyanate selected from the group consisting of ethyl lysine diisocyanate, hexamethylene diisocyanate, cyclohexyl diisocyanate.

In one embodiment, the isocyanate component comprises one or more isocyanates that are relatively non-absorbable. In one embodiment, the isocyanate is an aromatic isocyanate selected from diphenylmethanediisocyanate (MDI), including mixtures thereof such as mixtures of 2,4′-diphenylmethanediisocyanate and 4,4′-diphenylmethanediisocyanate isomers (ISONATE 50 OP, Dow Chemical Co. and RUBINATE 9433, Huntsman Corp.) and its pure 4,4-diphenylmethanediisocyanate form (MONDUR M, Bayer AG and RUBINATE 44, Huntsman Corp.). In one embodiment, the aromatic isocyanate is one of the commercially available polymeric isocyanates (e.g., polycarbodiimide-modified diphenylmethane disocyanate (ISONATE 143L) and polymethylene polyphenylisocyanate that contains MDI (ISONATE PAPI 901 or ISONATE PAPI 27) (Dow Chemical Co.)). These isocyanates, particularly the diphenylmethane derivatives, generally result in non-absorbable or slowly absorbable polyurethanes.

In embodiments, the isocyanate component comprises or consists of a resorbable lactyl diisocyanate, preferably benzoic acid, 4-isocyanato-1,1′-[oxybis[2,1-ethanediyloxy(1-methyl-2-oxo-2,1-ethanediyl)]]ester, referred to herein as absorbable lactide diester (ALD). In one embodiment in which the composition is fully or partially absorbable, the isocyanate component comprises or consists of [5-[2-[2-(4-Isocyanatobenzoyl)oxypropanoyloxy]-ethoxy]-1-methyl-2-oxo-pentyl]-4-isocyanatobenzoate, or “ALD”. In one embodiment, the two lactyl moieties of ALD each are racemic. Alternatively, these lactyl moieties may both have D or the L conformations. Alternatively, one lactyl moiety may be D while the other is L, or one may be D, L while the other is D or L. Such changes in stereochemistry may improve the physical and/or biological properties of the resulting polymer.

In one embodiment, the adduct of an isocyanate is selected from a hexamethylene diisocyanate trimer (DESMODUR N-3390) and a hexamethylene diisocyanate biuret (DESMODUR N-100) both commercially available from Bayer AG.

In one embodiment, the settable composition, which may be formed from a polymer selected from a polyurethane, a polyureaurethane, a polyetherurethane, or a polyesterurethane comprises at least one hydrolysable linkage. In one embodiment, the at least one hydrolysable linkage is derived from glycolic acid, lactic acid, caprolactone, or p-dioxanone. In one embodiment, the at least one hydrolysable linkage is selected from the group consisting of ester, amide, anhydride and sulfonamide linkages between the ester-urethane, urethane- or ureaurethane-containing groups. In one embodiment, the composition comprises one or more glycolyl, lactyl, or caprolactyl hydrolysable ester linkages. In one embodiment, the composition comprises one or more ethylene glycol, diethylene glycol, propane diol or butane diol hydrolysable ester linkages. In one embodiment, the composition comprises one or more ethylene diamine, propane diamine, butane diamine, hexamethylene diamine and polyalkylene diamine hydrolysable amide linkages. In one embodiment, the composition comprises one or more lactyl hydrolysable ester linkages and each asymmetric lactyl moiety present in the polymer is selected from one or more of the D, the L or the DL (racemic) stereoisomers.

The hydrolysable isocyanate based compositions are degradable at least due to the presence of functional groups in the polymer chain that are readily hydrolysable under physiological conditions. Thus, the term “partially degradable” as used in the present specification encompasses the percentage of functional groups in the polymer chain that are hydrolyzed compared to the total number of hydrolysable groups. In this context, a partially degradable isocyanate based composition encompasses compositions in which, after a suitable period of time, about 75% of the hydrolysable groups are hydrolyzed. In certain embodiments, a partially degradable compositions is one in which about 25% to 75% (i.e., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75% or 25-30, 25-35, 25-40, 25-45, 25-50, 25-55, 25-60, 25-65, 25-70, 30-35, 30-40, 30-45, 35-50, 35-55, 35-60, 35-65, 35-70, 35-75, 40-45, 40-50, 40-55, 40-60, 40-65, 40-70, 40-75, 45-50, 45-55, 45-60, 45-65, 45-70, 45-75, 50-55, 50-60, 50-65, 50-70, 50-75, 55-60, 55-65, 55-70, 55-75, 60-65, 60-70, 60-75, 65-70, 65-75, or 70-75%) or 50% to 75% (i.e., 50, 51, 52, 53, 54, 55, 56, 57, 58, 59, 60, 61, 62, 63, 64, 65, 66, 67, 68, 69, 70, 71, 72, 73, 74, or 75% or 50-55, 50-60, 50-65, 50-70, 55-60, 55-65, 55-70, 55-75, 60-65, 60-70, 60-75, 65-70, 65-75, or 70-75%) or about 75% to 90% (i.e., 75, 76, 77, 78, 79, 80, 81, 82, 83, 84, 85, 86, 87, 88, 89, or 90% or 75-80, 75-85, 75-90, 80-85, 80-90, or 85-90%)) of the hydrolysable groups are hydrolyzed.

The rate of degradation of the compositions can be controlled in order to provide compositions that degrade at a slower or faster rate, compared to a base composition. In general, the rate of degradation is controlled by varying the isocyanate and polyol/polyamine components of the compositions, as well as the optional chain extender component according to the following parameters. In one aspect, the rate of degradation is controlled by choice of the isocyanate and polyol. Generally, the more hydrolysable linkages, the faster it will degrade while less hydrolysable linkages will degrade slower. In another aspect, the rate of degradation is controlled by varying the hydrophobic/hydrophilic balance of the polyol/polyamine component. Generally, the more carbon atoms or methylene groups between the hydrolysable functions, the slower will be the hydrolysis. For example, ethylene glycol will provide a composition that hydrolyses more rapidly than, for example, 1,3 propane diol, which in turn hydrolyses more rapidly than 1,4 butane diol. In addition, the use of hydrolysable diamines as chain extenders may increase the rate of hydrolysis. In another aspect, copolymers of caprolactone and glycolide hydrolyze faster than copolymers of caprolactone and lactide and the addition of D, L-lactide also increases the rate of hydrolysis. Thus, for example, a bis-diphenyldiisocyanate bridged with a polyglycolide, a polyglycolide-co-lactide, a polylactide, a polycaprolactone-co-glycolide, a polycaprolactone-co-lactide, a polycaprolactone will hydrolyze at increasingly slower rates. For comparison, polyurethanes prepared using methylene bis-diphenyldiisocyanate, with no hydrolyzable linkages, are not significantly degradable under physiological conditions. In other embodiments, enzymatic sensitive sites such as di or polylysines or arginines are incorporated into one or more of the substituents. In another embodiment, the polyol or polyamine component, e.g., hydroxymethylglycolate, may have a hydrolysable linkage to increase the rate of degradation.

In one embodiment, the isocyanate component comprises a polyaromatic di- or polyisocyanate having at least one hydrolysable linkage bridging at least two of the aromatic rings. In certain embodiments, the hydrolysable linkage bridging the aromatic rings is derived from glycolic acid, lactic acid, caprolactone, or p-dioxanone. In most cases, the hydrolyzable linkage is an ester which may degrade into an acid and an alcohol as a result of exposure to water or to naturally occurring esterases. Amide linkages are usually more difficult to hydrolyze than esters. Another option is the easily hydrolyzable acid anhydride linkage. Sulfonamides may also be considered in this context. The polyaromatic di- or polyisocyanates described herein are distinct from isocyantes having only a single aromatic ring such as toluene diisocyante, methylene bis-p-phenyl diisocyanate, and aromatic polyisocyanates generally. Suitable isocyanates are described in U.S. Pat. No. 7,772,352 and U.S. Patent Application Publication No. 2009/0292029, each of which is incorporated herein by reference.

In one embodiment, the isocyanate based compositions are formed from an isocyanate component that comprises or consists of a glycolide-linked polyaromatic diisocyanate monomer and a polyol component that comprises or consists of a polycaprolactone-co-glycolide polyol. In one embodiment, the isocyanate based compositions are formed from a reaction that also comprises butanediol, e.g., as a chain extender. In one embodiment, the composition is formed from a reaction that further comprises one or more of water, a carboxylic acid, e.g., benzoic acid (as a foaming agent), a divalent or polyvalent metal salt, a metal carbonate or bicarbonate, or a phosphate, e.g., for osteoconductivity. In one embodiment, the glycolide-linked diisocyanate monomer has the following structure:

OCN-φ-OCH2CO2CH2CH2OCH2CH20COCH2O-φ-NCO
para para

In one embodiment, the polycaprolactone-co-glycolide polyol has the following structure:

[HOCH₂CO₂CH₂CH₂CH₂CH₂CO₂CH₂OH]ₙ

In one embodiment, the isocyanate based compositions are formed from an isocyanate component that comprises or consists of a lactide linked diisocyanate monomer and a polyol component that comprises or consists of a polycaprolactone-co-lactide polyol. In one embodiment, the isocyanate based compositions are formed from a reaction that also comprises butanediol, e.g., as a chain extender. In one embodiment, the composition is formed from a reaction that further comprises one or more of water, a carboxylic acid, e.g., benzoic acid (as a foaming agent), a divalent or polyvalent metal salt, a metal carbonate or bicarbonate, or a phosphate, e.g., for osteoconductivity. In one embodiment, the lactide-linked diisocyanate monomer has the following structure:

OCN-φ-CO₂CH(CH₃)CO2CH2CH2OCH2CH20COCH(CH₃)CO₂-φ-NCO
para para

In one embodiment, the polycaprolactone-co-lactide polyol has the following structure:

HO[CH(CH₃)CO₂CH₂CH₂CH₂CH₂CO₂CH(CH₃)]OHₙ

### The Polyol/Polyamine Component

The diols, polyols, and polyamines suitable for use in forming absorbable polyurethane-based compositions are either degradable or non-degradable, or a mixture of the two. As used herein, the term “polyol” is meant to refer generically to diols and polyols, unless indicated otherwise. Generally, absorbable isocyanate based compositions are formed by the combination of an excess of the isocyanate component with the polyol/polyamine component. The relative amounts are calculated as the molar ratio of NCO groups of the isocyanate component (I) to the active hydrogen functional groups (H) (e.g., hydroxyl, amino, and mixtures thereof) of the polyol/polyamine component. Generally, the ratio of polyisocyanate to polyol/polyamine (I:H) is at least 0.5:1. In certain embodiments, the ratio is about 1:1, about 1.5:1, about 2:1, about 3:1, or about 4:1. In other embodiments, the ratio is about 5:1, about 8:1, about 10:1, about 20:1, or about 50:1.

In certain embodiments, the polyol/polyamine component is present in an isocyanate prepolymer in an amount of from about 0.5% to about 50% (i.e., 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% or 0.5-1, 0.5-5, 0.5-10, 0.5-15, 0.5-20, 0.5-25, 0.5-30, 0.5-35, 0.5-40, 0.5-45, 5-10, 5-15, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50%) by weight of the prepolymer. In certain embodiments, the polyol/polyamine component is present in an amount of from about 0.5% to 10% (i.e. 0.5, 0.6, 0.7, 0.8, 0.9, 1, 2, 3, 4, 5, 6, 7, 8, 9, or 10% or 0.5-1, 0.5-2, 0.5-3, 0.5-4, 0.5-5, 0.5-6, 0.5-7, 0.5-8, 0.5-9, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, or 8-10%), from about 10% to 20% (i.e., 10, 11, 12, 13, 14, 15, 16, 17, 18, 19, or 20% or 10-15, or 15-20), from about 20% to 35% (i.e., 20, 21, 22, 23, 24, 25, 26, 27, 28, 29, 30, 31, 32, 33, 34, or 35% or 20-25, 20-30, 25-30, 25-35, or 30-35%), from about 25% to 40% (i.e., 25, 26, 27, 28, 29, 30, 31, 32, 33, 34, 35, 36, 37, 38, 39, or 40% or 25-30, 25-35, 30-35, 30-40, or 35-40%), or from about 35% to 50% (i.e., 35, 36, 37, 38, 39, 40, 41, 42, 43, 44, 45, 46, 47, 48, 49, or 50% or 35-40, 35-45, 40-45, 40-50, or 45-50%) by weight of the prepolymer.

Polyols suitable for use include biocompatible, naturally occurring polyols, synthetic polyols, and mixtures thereof. In certain embodiments, the polyols comprise at least one ester group. In certain embodiments, the polyol comprises 2 to 4 (i.e., 2, 3, or 4) ester groups or 5 to 10 (i.e., 5, 6, 7, 8, 9, or 10) ester groups. In one embodiment, the polyol has two or more hydroxyl groups. Suitable polyols include diols and polydiols having repeating units containing up to about 18 carbon atoms. Examples of suitable diols include 1,2-ethanediol (ethylene glycol), 1,2-propanediol (propylene glycol), 1,3-propanediol, 1,4-butanediol, 1,5-pentanediol, 1,3-cyclopentanediol, 1,6-hexanediol, 1,8-octanediol and combinations thereof. Examples of preferred polydiols include polyethylene glycol with molecular weights of from about 500 to about 10000 (i.e., 500, 1000, 1500, 2000, 2500, 3000, 3500, 4000, 4500, 5000, 5500, 6000, 6500, 7000, 7500, 8000, 8500, 9000, 9500, or 10000 or 500-1000, 500-5000, 1000-5000, 1000-10000, or 5000-10000), polytetramethylene ether glycols, polyols derived from glycolide, lactide, trimethylenecarbonate, p-dioxanone and/or caprolactone with molecular weights of about 500 to about 10000 (i.e., 500, 1000, 1500, 2000, 2500, 3000, 3500, 4000, 4500, 5000, 5500, 6000, 6500, 7000, 7500, 8000, 8500, 9000, 9500, or 10000 or 500-1000, 500-5000, 1000-5000, 1000-10000, or 5000-10000).

In one embodiment, one or more alkylpyrrolidones (see e.g., U.S. Pat. No. 7,955,616) may be added to the polyol component to improve healing.

In one embodiment, the polyol is a synthetic polyol selected from a polycaprolactone polyol, polyester polyols, polyadipate polyols (e.g., poly(hexane-adipate) diol, poly(butane-adipate) diol, poly(ethylene/propylene-adipate) diol, poly(hexane/adipate/isophthalate diol)), and polyols that have been derived from a synthetic acid (e.g., isophthalic acid, maleic acid). An example of a suitable biocompatible synthetic polyol is a polycaprolactone diol that is commercially available from Dow Chemical under the trade name TONE 32 B8. Further non-limiting examples of suitable synthetic polyols include poly(oxypropylene) glycols, poly(oxytetramethylene) glycols, and poly(oxyethylene) glycols. In one embodiment, the synthetic polyol is selected from a polycaprolactone co-glycolide or a polycaprolactone co-lactide.

In one embodiment, the polyol is a naturally occurring polyol selected from castor oil and lesquerella oil, the polyols that may be obtained by chemical modification of naturally occurring vegetable oils (e.g., castor oil, olive oil, sesame oil, corn oil), naturally occurring oils that have been trans-esterified (e.g., a modified castor oil polyol that has been prepared by the transesterification reaction of natural castor oil with suitable crosslinkers (e.g., glycerol, trimethylolpropane, and the like) or with acids (such as adipic acid), and naturally occurring oils that have been hydrogenated. Further non-limiting examples of suitable naturally occurring polyols include the commercially available castor-oil-based polyols CASPOL5001, CASPOL1962, and CASPOL5004 (all available from CasChem, Inc.). In certain embodiments, the polyol is not a naturally occurring polyol such as castor oil and lesquerella oil.

In certain embodiments, an isocyanate prepolymer is combined with a polyamine to form a poly(urethane-urea). The polyamine may be a primary or secondary di-amine, or a hindered amine. Non-limiting examples of suitable polyamines include, hindered diamine (e.g., isophorone diamine, “IPDA”), 1,4-cyclohexyl diamine, 1,3-pentane diamine, and aliphatic secondary diamines, and mixtures thereof. In certain embodiments, aliphatic diamines and cycloaliphatic diamines may be particularly suitable, and may offer improved biocompatibility. Commercially available examples of suitable polyamines include CLEARLINK 1000 (Dorf Ketal).

Amines including diamines that may be suitable for use in the preparation of polyurea and polyureaurethanes include but are not limited to polyethyleneimines, PEG amines with weight average molecular weights from about 500 to about 5,000 (i.e., 500, 1000, 1500, 2000, 2500, 3000, 3500, 4000, 4500, or 5000 or 500-1000, 500-5000, or 1000-5000), polyoxypropylenediamines available under the tradename JEFFAMINES (Huntsman Corporation, Houston, Tex.) and polyetherdiamines in general, spermine, spermidine, hexamethylenediamine, octamethylenediamine, decamethylenediamine, dodecamethylenediamine, hexadecamethylenediamine, octadecamethylenediamine, polyamidoamine dendrimers, dextrans, PEG-dextran conjugates, cysteines, proteins and peptides containing amines, non-biologically active symmetrical and asymmetrical diamino compounds containing saturated and unsaturated, substituted and unsubstituted alkyl, aryl and alkylaryl groups having from about 2 to about 18 carbon atoms. (i.e., 2, 3, 4, 5, 6, 7, 8, 9, 10, 11, 12, 13, 14, 15, 16, 17, or 18 carbon atoms). Further, the diamino compound can be synthesized containing a hydrolyzable link such as one or more ester groups to accelerate the rate of polymer degradation (absorption) in the body. The following structure exemplifies this concept for hexamethylenediamine: H₂NCH₂CH₂CH₂COOCH₂CH₂NH₂.

In certain embodiments, the polyol comprises 2 to 4 (i.e., 2, 3, or 4) ester groups or 5 to 10 (i.e., 5, 6, 7, 8, 9, or 10) ester groups. Suitable polyols have at least two hydroxyl groups. In certain embodiments, the polyol has three or more hydroxyl groups making them crosslinkers.

### The Chain-Extender/Crosslinker Component

In certain embodiments, one or more optional chain extenders or crosslinkers is incorporated in the formation of the absorbable isocyanate-based compositions. In certain embodiments, only a chain extender is present. In other embodiments, both a chain extender and a crosslinker are present. In one embodiment, the one or more chain extenders is a low molecular weight polyhydroxyl- and/or polyamine-terminated compound having a molecular weight in the range of 10 to 500 (i.e., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 or 10-100, 10-200, 10-300, 10-400, 100-200, 100-300, 100-400, 100-500, 200-300, 200-400, 200-500, 300-400, 300-500, or 400-500) Daltons and a functionality of at least two. In certain embodiments, the chain extender is a short-chain diol or diamine. In a particular embodiment, the chain extender or crosslinker is selected from glycerol, 1,4 butanediol, 1,6-hexanediol, diethylene glycol, and combinations thereof. Chain extenders having a functionality of three or more than three are also referred to as crosslinkers. In certain embodiments, the compositions described herein are formed without crosslinkers and the compositions are not crosslinked. In other embodiments, the compositions are formed with one or more crosslinkers. The degree of crosslinking can be controlled, for example, by varying the amount of crosslinker present.

In certain embodiments, the chain-extender or crosslinker is present in an isocyanate prepolymer in an amount in the range of about 5% to about 80% (i.e., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80% or 5-10, 5-15, 5-20, 5-25, 5-30, 5-35, 5-40, 5-45, 5-50, 5-55, 5-60, 5-65, 5-70, 5-75, 10-15, 10-20, 10-25, 10-30, 10-35, 10-40, 10-45, 10-50, 10-55, 10-60, 10-65, 10-70, 10-75, 10-80, 15-20, 15-25, 15-30, 15-35, 15-40, 15-45, 15-50, 15-55, 15-60, 15-65, 15-70, 15-75, 15-80, 20-25, 20-30, 20-35, 20-40, 20-45, 20-50, 20-55, 20-60, 20-65, 20-70, 20-75, 20-80, 25-30, 25-35, 25-40, 25-45, 25-50, 25-55, 25-60, 25-65, 25-70, 25-75, 30-35, 30-40, 30-45, 30-50, 30-55, 30-60, 30-65, 30-70, 30-75, 30-80, 35-40, 35-45, 35-50, 35-55, 35-60, 35-65, 35-70, 35-75, 35-80, 40-45, 40-50, 45-55, 45-60, 45-65, 45-75, 45-80, 50-55, 50-60, 50-65, 50-70, 50-75, 50-80, 55-60, 55-65, 55-70, 55-75, 55-80, 60-65, 60-70, 60-75, 60-80, 65-70, 65-75, 65-80, 70-75, 70-80, or 75-80%) by weight of the isocyanate prepolymer. In certain embodiments, the chain-extender or crosslinker is present in an amount of from about 5% to 20% (i.e., 5, 6, 7, 8, 9, 10, 11, 12, 13, 14, 15, 16, 17, 18, 19, or 20% or 5-10, 5-15, 10-15, 10-20, or 15-20%), about 20% to 30% (i.e., 20, 21, 22, 23, 24, 25, 26, 27, 28, 29, or 30% or 20-25 or 25-30%), about 30% to 40% (i.e., 30, 31, 32, 33, 34, 35, 36, 37, 38, 39, or 40% or 30-35 or 35-40%), about 40% to 50% (i.e., 40, 41, 42, 43, 44, 45, 46, 47, 48, 49, or 50% or 40-45 or 45-50%), about 50% to 60% (i.e., 50, 51, 52, 53, 54, 55, 56, 57, 58, 59, or 60% or 50-55 or 55-60%), from about 60% to 70% (i.e., 60, 61, 62, 63, 64, 65, 66, 67, 68, 69, or 70% or 60-65 or 65-70%), or from about 70% to 80% (i.e., 70, 71, 72, 73, 74, 75, 76, 77, 78, 79, or 80% or 70-75 or 75-80%) by weight of the isocyanate prepolymer.

The chain extender may be degradable or non-degradable. Preferably, at least one degradable chain extender is used. Suitable degradable chain extenders for use in any of the compositions described herein are described in U.S. Patent Application Serial No. 2009/0082540, which is incorporated herein by reference. In one embodiment, the at least one degradable chain extender is HOCH₂CO₂CH₂CH₂OH or HOCH₂CO₂CH₂CH₂O₂CCH₂OH.

Other suitable chain-extenders or crosslinkers include natural or synthetic aliphatic polyols. Suitable polydiols for use in the compositions described herein include diol or diol repeating units with up to 8 carbon atoms. Non-limiting examples include 1,2-ethanediol (ethylene glycol), 1,2-propanediol (propylene glycol), 1,3-propanediol, 1,4-butanediol, 1,5-pentanediol, 1,3-cyclopentanediol, 1,6-hexanediol, 1,4-cyclohexanediol, 1,8-octanediol and combinations thereof.

In other embodiments, the chain extender is a polyol selected from polyethylene glycol and polypropylene glycol having molecular weights of 500-10000 (i.e., 500, 1000, 1500, 2000, 2500, 3000, 3500, 4000, 4500, 5000, 5500, 6000, 6500, 7000, 7500, 8000, 8500, 9000, 9500, or 10000 or 500-1000, 500-5000, 500-10000, 1000-5000, 1000-10000, or 5000-10000) Daltons. Other examples include CASPOL1962 and CASPOL5004. In certain embodiments the preferred polydiols include polydiols selected from polyethylene glycol and polypropylene glycol with molecular weights of 500-10000 (i.e., 500, 1000, 1500, 2000, 2500, 3000, 3500, 4000, 4500, 5000, 5500, 6000, 6500, 7000, 7500, 8000, 8500, 9000, 9500, or 10000 or 500-1000, 500-5000, 500-10000, 1000-5000, 1000-10000, or 5000-10000). In some embodiments, the crosslinker is a non-absorbable crosslinker selected from triethanolamine (TEA), trimethylolpropane, and QUADROL (BASF Corp.). In some embodiments, the chain-extender is a non-degradable chain extender selected from 1,4-butanediol, 1,6-hexanediol, and diethylene glycol. The chain-extender or crosslinker may be present in an isocyanate prepolymer in an amount in the range of about 10% to about 80% by weight of the isocyanate prepolymer.

In another embodiment, the dual putty system is able to set and adhere in aqueous environments. By nature, the isocyanate component, even containing hydrolysable linkages, is essentially hydrophobic and will resist dissolution in aqueous systems. This is true for diamines in this context. It has been found that making the diol more hydrophobic by adding a hydrophobic hydrocarbon-rich residue to a polyol, e.g., glyceryl-1 or 2-monostearate, a more water resistant system is obtained. A variation of this embodiment involves the substitution of a silicon-based moiety for the hydrocarbon-rich residue although this may affect absorbability. Alternatively hydrophobicity and setting rate in aqueous environments can be improved through the use of hydrophobic fillers such as insoluble or weakly soluble aliphatic molecules and salts thereof, including divalent salts, (e.g., calcium, magnesium, or zinc) of fatty acids. Also useful are cholesterol and its derivatives, as well as silated derivatives of ceramics or bone (Shimp et al., U.S. Pat. No. 7,270,813). Another embodiment of a water resistant, settable, dual putty system adds a small amount of hydrophobic isocyanate to the relatively hydrophilic polyol component resulting in a water-resistant mixture of polyol containing a minor amount of hydrophobic polyurethane prepolymer. In one embodiment, the chain extender does not comprise an amino acid group.

The settable compositions described herein may contain optional particulate materials. In one embodiment, the particulate material is an osteoconductive material. In certain embodiments, the particulate material supports or promotes the growth of bone at the application site. In one embodiment, the particulate material is non-resorbable. In certain embodiments, the mean particle size of the optional particulate material is in the micron or submicron range. In one embodiment, the mean particle size is from about 0.001 to 0.100 microns, from about 0.100 to 5 microns, from about 5 to 100 microns, from about 5 to 500 microns, or from about 500 to 2000 microns.

In one embodiment, the optional particulate material is a carbonate or bicarbonate material. In one embodiment, the carbonate or bicarbonate material comprises or consists of one or more of calcium carbonate, magnesium carbonate, aluminum carbonate, iron carbonate, zinc carbonate, calcium bicarbonate, and sodium bicarbonate. In one embodiment, the optional particulate material comprises or consists of bone (e.g., demineralized bone, bone morphogenetic protein, allograft bone, and/or autogenous bone), calcium phosphate, siliconized calcium phosphate, substituted calcium phosphates (e.g., with magnesium, strontium, or silicate), calcium pyrophosphate, hydroxyapatite, polymethyl methacrylate, glass-ionomer, absorbable phosphate glass, calcium sulfate, tricalcium phosphate (e.g., beta tricalcium phosphate), barium sulfate, a calcium, magnesium, zinc, aluminum or barium salt of lauric acid, a calcium, magnesium, zinc, aluminum or barium salt of myristic acid; a calcium, magnesium, zinc, aluminum or barium salt of palmitic acid, a calcium, magnesium, zinc, aluminum and/or barium salt of stearic acid, a calcium, magnesium, zinc, aluminum or barium salt of arachidic acid, a calcium, magnesium, zinc, aluminum or barium salt of behenic acid, or any combination of the foregoing. Other examples include one or more poly ether ether ketones (e.g., PEEK, REPLACE (Cortek, Inc.), EXPANCEL (Akzo Nobel)). In other embodiments, the particulate material is a ceramic such as substituted calcium phosphates (e.g, silicate, strontium or magnesium substitution) or a glass such as bioglass. In one embodiment, the particulate material comprises or consists of one or more of calcium sulfate, calcium phosphosilicate, sodium phosphate, calcium aluminate, calcium phosphate, hydroxyapatite, demineralized bone, or mineralized bone.

The optional particulate material, when present, may comprise any one or more of the materials listed in the embodiments above. In one embodiment, the particulate material, if present in the composition, does not comprise calcium carbonate. In one embodiment, the particulate material may be polymeric such as a polyurethane.

In one embodiment, the particulate material is present in an amount of from about 0.01% to about 10% (i.e., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10% or 0.01-1, 0.01-2, 0.01-3, 0.01-4, 0.01-5, 0.01-6, 0.01-7, 0.01-8, 0.01-9, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10%) by weight of the composition. In certain embodiments, the optional particulate material is present in an amount of 0.10% to 10% (i.e., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10% or 0.1-1, 0.1-2, 0.1-3, 0.1-4, 0.1-5, 0.1-6, 0.1-7, 0.1-8, 0.1-9, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10%), 1% to 10% (i.e., 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10% or 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10 or 9-10%), or 5% to 10% (i.e., 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10% or 5-6, 5-7, 5-8, 5-9, 6-7, 6-8, 6-9, 6-10, 7-8, 7-9, 7-10, 8-9, 8-10, or 9-10%). In other embodiments, the optional particulate material is present in an amount of from about 10% to about 20% (i.e., 10, 11, 12, 13, 14, 15, 16, 17, 18, 19, or 20% or 10-15 or 15-20%) by weight of the composition, or from about 20% to 30% (i.e., 20, 21, 22, 23, 24, 25, 26, 27, 28, 29, or 30% or 20-25 or 25-30%), about 30% to 40% (i.e., 30, 31, 32, 33, 34, 35, 36, 37, 38, 39, or 40% or 30-35 or 40-45%), about 40% to 50% (i.e., 40, 41, 42, 43, 44, 45, 46, 47, 48, 49, or 50% or 40-45 or 45-50%), about 50% to 60% (i.e., 50, 51, 52, 53, 54, 55, 56, 57, 58, 59, or 60% or 50-55 or 55-60%), about 60% to 70% (i.e., 60, 61, 62, 63, 64, 65, 66, 67, 68, 69, or 70% or 60-65 or 65-70%) or about 70% to 80% (i.e., 70, 71, 72, 73, 74, 75, 76, 77, 78, 79, or 80% or 70-75 or 75-80%) by weight of the composition.

In one embodiment, the particulate additive material is graphene (available from Applied Graphene Materials and Thomas Swan, Ltd.), a single atomic layer of graphite that is electrically conductive, highly elastic, is about 100 times stronger than steel and which may be of value improving the quality of tissue healing and new bone stimulation.

### Other Optional Additives

The compositions may also optionally comprise one or more “cell openers.” Non-limiting examples include ORTOGEL501 (Goldschmidt) (an anti-foaming additive) and X-AIR (Specialty Polymers & Services). In certain embodiments, the cell openers are present in an amount in of from about 0.1% to 5% (i.e., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5% or 0.1-1, 0.1-2, 0.1-3, 0.1-4, 1-3, 1-4, 1-5, 2-4, 2-5, or 1-2, 1-3, 1-4, 1-5, 2-3, 2-4, 2-5, 3-4, 3-5, or 4-5%) by weight of the composition. In one embodiment, the cell openers are present in an amount in of from about 1% to 2% or 1% to 3% by weight of the composition. Optional additives can be added to the magnesium based section but for the malonate/cyanoacrylate section, no active hydrogen atoms can be present including those in water because they will initiate polymerization.

The compositions may also optionally comprise one or more therapeutic agents. In one embodiment, the one or more therapeutic agents are selected from an anti-cancer agent, an antimicrobial agent, an antibiotic, a local anesthetic or analgesic, a statin and an anti-inflammatory agent. In one embodiment, the antibiotic is selected from a broad spectrum antibiotic, such as gentamicin, clindamycin, and erythromycin, or a gram positive and gram negative family antibiotic such as an ampicillin and a cephalosporin. In one embodiment, the local anesthetic or analgesic is selected from lidocaine, bupivacaine, tetracaine, and ropivacaine. In one embodiment, the local anesthetic or analgesic is selected from lidocaine, benzocaine and fentanyl (a potent non-opioid anesthetic). In one embodiment, the one or more anti-inflammatory substances is selected from a non-specific anti-inflammatory such as ibuprofen and aspirin, or a COX-2 specific inhibitor such as rofecoxib and celeboxib.

In one embodiment, component A is a putty comprised of a concentrated aqueous solution of a polyanionic polymer, e.g., carboxymethylcellulose, and component B is a putty comprised of a concentrated aqueous solution of a polycationic polymer. e.g., chitosan, either of which may be optionally crosslinked. The combined materials are hemostatic when applied to a bleeding surface.

In one embodiment, the compositions further comprise one or more of an antioxidant, a colorant, a steroid, calcium stearate, tocopheryl acetate, and triacetin. In one embodiment, the antioxidant is selected from IRGANOX 1010 and IRGANOX 1035 (Ciba Geigy), and CYANOX 1790 and CYANOX 2777 (Cytec Industries). In certain embodiments, the antioxidant is present in an amount of from about 0.01% to 0.5% (i.e., 0.01, 0.02, 0.03, 0.04, 0.05, 0.06, 0.07, 0.08, 0.09, 0.1, 0.2, 0.3, 0.4, or 0.5% or 0.01-0.1, 0.01-0.2, 0.01-0.3, 0.01-0.4, 0.01-0.5, 0.1-0.2, 0.1-0.3, 0.1-0.4, 0.1-0.5, 0.2-0.3, 0.3-0.4, 0.3-0.5, or 0.4-0.5%) by weight of the composition. In one embodiment, the composition comprises one or more of calcium stearate, tocopheryl acetate, and triacetin, each present in a component putty of the composition in an amount ranging from 0.1 to 5% (i.e., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5% or 0.1-1, 0.1-2, 0.1-3, 0.1-4, 1-2, 1-3, 1-4, 1-5, 2-3, 2-4, 2-5, 3-4, 3-5, or 4-5%) based upon the weight of the component putty. Non-limiting examples of colorants that may be included in the compositions are gentian violet, D&C Violet #2, and D&C Green #6.

In one embodiment, the steroid is a steroid-based compound, such as an intracellular messenger, effective to modulate the rate of tissue growth, including bone growth.

In one embodiment, the compositions further comprise one or more growth factors, for example BMP-2, BMP-7, PDGF, EGF, etc.

As used herein, the terms “Pluronics” and “polaxmers” are used interchangeably herein to refer to nonionic triblock copolymers containing a central hydrophobic chain of polyoxypropylene flanked by two hydrophilic chains of polyoxyethylene. The name of a given Pluronic starts with a letter to define its physical form at room temperature (L=liquid, P=paste, F=flake (solid)) followed by a two or three digit number. The first digit (or two digits in a three-digit number) multiplied by 300 indicates the approximate molecular weight of the hydrophobe, and the last digit multiplied by 10 gives the percentage polyoxyethylene content.

### Non-Absorbable Putties

Examples of non-absorbable putties that can be used in the method 10 or with any of the molds described herein can be found in PCT Application No. PCT/US2022/038801, filed Jul. 29, 2022, published as WO 2023/014592, and entitled “Nonabsorbable Settable Multi-Putty Cements, Homeostatic Compositions, and Methods of Use,” which is incorporated reference herein in its entirety.

Non-absorbable putties include settable, nonabsorbable polyurethane, polyureaurethane, polyetherurethane or polyetherureaurethane compositions. The settable, nonabsorbable polyurethane, polyureaurethane, polyetherurethane or polyetherureaurethane compositions can be made by combining a polyisocyanate or a polyisocyanate-based prepolymer component with a polyol and/or polyamine component, to form a polyurethane, polyureaurethane, polyetherurethane or polyetherureaurethane-based composition. In some embodiments, the polyisocyanate component and the polyol and/or polyamine component can be combined with one or more chain extenders as described herein.

The settable, nonabsorbable polyurethane, polyureaurethane, polyetherurethane or polyetherureaurethane compositions are not absorbable in vivo, but are biocompatible and suitable for use in vivo, especially for use in bone repair and replacement surgery. The settable, nonabsorbable polyurethane, polyureaurethane, polyetherurethane or polyetherureaurethane compositions are suitable for use as a bone cement, bone substitute, and/or a bone hemostatic agent. The term “biocompatible” as used throughout herein refers to materials that do not induce adverse side effects when administered or implanted in vivo. The term biocompatible may be used interchangeably with “nontoxic” herein. The terms “nonabsorbable”, “nondegradable”, and “permanent”, as used throughout herein, are used to describe the persistence of the device once implanted in vivo, and are used interchangeably to refer to the resistance to degradation.

Non-absorbable Putties can be formed from a sterile, settable, nonabsorbable composition comprising a set of at least two reactive putties, A and at least B (i.e., a putty A and at least a putty B), which can be hand-mixed and wherein the putties comprise amounts of reagents which react and cure into a final, hardened form selected from the group consisting of polyurethane, polyureaurethane, polyetherurethane and polyetherureaurethane, over a period of time at room or body temperature, each putty being physically separated from the other putty of the composition; wherein putty A comprises of a polyisocyanate component, a polyol or a polyamine component, a non-hydrolysable crosslinker, a particulate material, and additive material(s); and wherein putty B comprises of a polyisocyanate component, a polyol and/or a polyamine component, a nonabsorbable cross-linker, a particulate material, and additive material(s). In some embodiments, the settable, nonabsorbable polyurethane, polyureaurethane, polyetherurethane or polyetherureaurethane compositions are formed by the reaction of one or more polyaromatic di- or polyisocyanates with one or more diols or polyols and/or diamines or polyamines. In some embodiments, the settable, nonabsorbable polyurethane, polyureaurethane, polyetherurethane or polyetherureaurethane compositions may also include the addition of an optional chain extender or crosslinker. In some embodiments, the settable, nonabsorbable polyurethane, polyureaurethane, polyetherurethane or polyetherureaurethane compositions are formed in the absence of a crosslinker.

Non-absorbable Putties can be formed from a settable, nonabsorbable composition comprising a set of at least two reactive putties, A and at least B, which, upon mixing together, react and cure into a final hardened form selected from the group consisting of polyurethane, polyureaurethane, polyetherurethane or polyetherueaurethane, over a period of time at room or body temperature, each putty being physically separated from the other putty of the composition; wherein putty A comprises 10-70% of a polyisocyanate component, 0-5% of a polyol or a polyamine component, 30-85% of one or more particulate material(s), and 0-8% of one or more additive material(s), based upon total weight of putty A; and wherein putty B comprises 0-5% of a polyisocyanate component, 5-80% of a polyol or a polyamine component, 30-95% of one or more particulate material(s), and 0-5% of one or more additive material(s), based upon the weight of putty B, wherein the polyol or polyamine component of putty A or putty B or both comprise one or more nonabsorbable, non-hydrolysable crosslinker(s).

In some embodiments of the composition of this disclosure, putty A comprises 25-60% of a polyisocyanate component, and 40-75% of one or more particulate materials(s); based upon the weight of putty A; and putty B comprises 30-70% of a polyol and/or a polyamine component, and 30-70% of one or more particulate material(s), based upon the weight of putty B.

In some embodiments of the composition of this disclosure, putty A comprises 25-60% (e.g., 25%-30%, 30%-35%, 35%-40%, 40%-45%, 45%-50%, 50%-55% or 55%-60%) of a polyisocyanate component, based upon total weight of putty A. In some embodiments of the composition of this disclosure, putty A comprises 40-75% (e.g., 40%-45%, 45%-50%, 50%-55%, 55%-60%, 60%-65%, 65%-70% or 70%-75%) of one or more particulate material(s), based upon total weight of putty A.

In some embodiments of the composition of this disclosure, putty B comprises 30-70% (e.g., 30%-35%, 35%-40%, 40%-45%, 45%-50%, 50%-55%, 55%-60%, 60%-65% or 65%-70%) of a polyol and/or a polyamine component, based upon total weight of putty B. In some embodiments of the composition of this disclosure, putty B comprises 30-70% (e.g., 30%-35%, 35%-40%, 40%-45%, 45%-50%, 50%-55%, 55%-60%, 60%-65% or 65%-70%) of one or more particulate material(s), based upon total weight of putty B.

In some embodiments of the composition of this disclosure, putty A comprises 15-40% of a polyisocyanate component, 2-3% of a polyol and/or a polyamine component, and 60-85% of one or more particulate material(s), based upon the weight of putty A; and putty B comprises 2-5% of a polyisocyanate compound, 15-25% of a polyol and/or a polyamine component, and 70-85% of one or more particulate material(s), based upon the weight of putty B.

In some embodiments of the composition of this disclosure, putty A comprises 15-40% (e.g., 15%-20%, 20%-25%, 25%-30%, 30%-35%, or 35%-40%) of a polyisocyanate component, based upon the total weight of putty A. In some embodiments of the composition of this disclosure, putty B comprises 2%-5% (e.g., 2%-2.5%, 2.5%-3%, 3%-3.5%, 3.5%-4%, 4%-4.5% or 4.5%-5%) of a polyisocyanate component, based upon the total weight of putty B.

In some embodiments of the composition of this disclosure, putty A comprises 2-3% (e.g., 2%-2.2%, 2.2%-2.4%, 2.4%-2.6%, 2.6%-2.8% or 2.8%-3%) of a polyol and/or a polyamine component, based upon the total weight of putty A. In some embodiments of the composition of this disclosure, putty B comprises 15-25% (e.g., 15-16%, 16%-17%, 17%-18%, 18%-20%, 20%-21%, 21%-22%, 22%-23%, 23%-24% or 24%-25%) of a polyol and/or a polyamine component, based upon the total weight of putty B.

In some embodiments of the composition of this disclosure, putty A comprises 60-85% (e.g., 60%-65%, 65%-70%4, 70%-75%, 75%-80% or 80%4-85%) of one or more particulate materials), based upon the total weight of putty A. In some embodiments of the composition of this disclosure, putty B comprises 70-85% (e.g., 70%-72%, 72%-74%, 74%-76%, 76%-80%, 80%-82% or 82%-85%) of one or more particulate material(s), based upon the total weight of putty B.

In some embodiments of the composition of this disclosure, putty A comprises 25-45% of a polyisocyanate component, 3-5% of a polyol and/or a polyamine component, and 50-65% of one or more particulate material(s), based upon the weight of putty A; and putty B comprises 2-5% of a polyisocyanate compound, 5-35% of a polyol and/or a polyamine component, and 60-92% of one or more particulate material(s), based upon the weight of putty B.

In some embodiments of the composition of this disclosure, putty A comprises 25-45% (e.g., 25%-30%, 30%-35%, 35%-40%, 40%-45% or 45%-50%) of a polyisocyanate component, based upon the total weight of putty A. In some embodiments of the composition of this disclosure, putty B comprises 2-5% (e.g., 2%-2.5%, 2.5%-3%, 3%-3.5%, 3.5%-4%, 4%-4.59% or 4.5%-5%) of a polyisocyanate component, based upon the total weight of putty B.

In some embodiments of the composition of this disclosure, putty A comprises 3-5% (e.g., 3%-3.5%, 3.5%-4%, 4%-4.5% or 4.5%-5%) of a polyol and/or a polyamine component, based upon the total weight of putty A. In some embodiments of the composition of this disclosure, putty B comprises 5-35% (e.g., 5%-10%, 10%-15%, 15%-20%, 20%-25%, 25%-30% or 30%-35%) of a polyol and/or a polyamine component, based upon the total weight of putty B.

In some embodiments of the composition of this disclosure, putty A comprises 50-65% (e.g., 50-52%, 52%-54%, 54%-56%, 56%-58%, 58%-60%, 60%-62% or 62%-65%) of one or more particulate material(s), based upon the total weight of putty A. In some embodiments of the composition of this disclosure, putty B comprises 60-92% (e.g., 60%-64%, 64%-68%, 68%-72%, 72%-76%, 76%-80%, 80%-84%, 84%-88% or 88%-92%) of one or more particulate material(s), based upon the total weight of putty B.

In some embodiments of the composition of this disclosure, putty A comprises 0-8% (e.g., 0-1%, 1%-2%, 2%-3%, 3%-4%, 4%-5%, 5%-6%, 6%-7% or 7%-8%) of one or more additive material(s), based upon the total weight of putty A.

In some embodiments of the composition of this disclosure, putty B comprises 0-5% (e.g., 0-1%, 1%-2%, 2%-3%, 3%-4% or 4%-5%) of one or more additive material(s), based upon the total weight of putty B.

In some embodiments of the composition of this disclosure, the polyisocyanate component of the putty A, the putty B, or both is a nonabsorbable polyisocyanate. In some embodiments of the composition of this disclosure, the polyisocyanate component of the putty A, the putty B, or both is a nondegradable polyaromatic isocyanate, preferably a diisocyanate or a polyisocyanate.

In some embodiments of the composition of this disclosure, the polyisocyanate component of the putty A, the putty B, or both comprises one or more one or more polyisocyanate(s). In some embodiments of the composition of this disclosure, the one or more polyisocyanate(s) is a non-hydrolysable polyisocyanate. In some embodiments of the composition of this disclosure, the one or more polyisocyanate(s) of the putty A, the putty B, or both is a diisocyanate, triisocyanate, or tetraisocyanate or a combination thereof. In some embodiments of the composition of this disclosure, the one or more polyisocyanate(s) of the putty A, the putty B, or both is 1,1,1-tris-(4-isocyanatophenoxymethyl)-propane (also referred to as trimethylolpropane-4-nitrophenyl, or TMPI).

In some embodiments of the composition of this disclosure, the polyamine component of the putty A, the putty B or both comprises one or more polyamine. In some embodiments of the composition of this disclosure, the one or more polyamine(s) of the putty A, the putty B, or both is ethylene diamine, propane diamine, butane diamine, cyclopentane diamine, cyclohexane diamine, or hexamethylene diamine, or a combination thereof.

In some embodiments of the composition of this disclosure, the polyol component of the putty A, the putty B or both comprises one or more polyol(s). In some embodiments of the composition of this disclosure, the one or more polyols of the putty A, the putty B, or both is a trimethylolpropane ethoxylate, triethanolamine, tetrakis (2-hydroxyethyl)ethylenediamine, or tetrakis (2-hydroxypropyl)ethylenediamine, or a combination thereof.

In some embodiments of the composition of this disclosure, the one or more nonabsorbable, non-hydrolysable cross-linker(s) or chain extender(s) of the putty A, the putty B, or both is an aliphatic polyol. In some embodiments of the composition of this disclosure, the one or more nonabsorbable, non-hydrolysable crosslinker(s) or chain extender(s) of the putty A, the putty B, or both is a trimethylolpropane ethoxylate, triethanolamine, glycerol, pentaerythritol, a trifunctional castor oil-based polyol, trimethylolpropane polyol, tetrakis(2-hydroxyethyl)ethylenediamine, or tetrakis(2-hydroxypropyl)ethylenediamine, or a combination thereof. In some embodiments of the composition of this disclosure, the trimethylolpropane ethoxylate is a trimethylolpropane ethoxylate (TMPE) of molecular weight 450, a TMPE of molecular weight 170, or a TMPE of molecular weight 1014.

In some embodiments of the composition of this disclosure, the polyisocyanate component and the polyol/polyamine component of the putty A, the putty B, or both of the settable, nonabsorbable polyurethane, polyureaurethane, polyetherurethane or polyetherureaurethane compositions are crosslinked. In some embodiments of the composition of this disclosure, the polyisocyanate component and the polyol/polyamine component the putty A, the putty B, or both of the settable, nonabsorbable polyurethane, polyureaurethane, polyetherurethane or polyetherureaurethane compositions are not crosslinked. In some embodiments, the putty A, the putty B, or both of the settable, nonabsorbable polyurethane, polyureaurethane, polyetherurethane or polyetherureaurethane compositions comprise an ether-linked tri- or tetra-isocyanate and a trimethylolpropane polyol. In some embodiments of the composition of this disclosure, the putty A, the putty B, or both of the settable, nonabsorbable polyurethane, polyureaurethane, polyetherurethane or polyetherureaurethane compositions further comprises triethanolamine as a chain extender. In some embodiments of the composition of this disclosure, the putty A, the putty B, or both of the settable, nonabsorbable polyurethane, polyureaurethane, polyetherurethane or polyetherureaurethane compositions further comprises a tetraol, such as tetrakis(2-hydroxyethyl)ethylenediamine or tetrakis(2-hydroxypropyl)ethylenediamine, as a chain extender and/or a reaction catalyst. In some embodiments of the composition of this disclosure, the putty A, the putty B, or both of the settable, nonabsorbable polyurethane, polyureaurethane, polyetherurethane or polyetherureaurethane compositions further comprises one or more of water, a carboxylic acid (e.g., lauric acid), and/or a divalent or polyvalent metal salt.

In some embodiments of the composition of this disclosure, the particulate material(s) of the putty A, the putty B, or both is a calcium phosphate, siliconized calcium phosphate, substituted calcium phosphates (substituted with magnesium, strontium, or silicate), calcium pyrophosphate, hydroxyapatite, polymethyl methacrylate, or tricalcium phosphate, or a combination thereof. In one embodiment, the one or more particulate materials of the putty A, the putty B, or both is present in an amount that is up to about 80 wt % of the final composition. In one embodiment, the one or more particulate material(s) is a carbonate or bicarbonate selected from calcium carbonate, magnesium carbonate, aluminum carbonate, iron carbonate, zinc carbonate, calcium bicarbonate, and sodium bicarbonate. In one embodiment, the one or more particulate materials do not comprise calcium carbonate or calcium phosphate. In one embodiment, the one or more particulate material(s) is selected from embedded particles of bone, demineralized bone, bone morphogenetic protein, hydroxyapatite, calcium phosphate, siliconized calcium phosphate, an inorganic material (e.g. stainless steel powder), a bone substitute material, a carbonate selected from magnesium carbonate, aluminum carbonate, iron carbonate, zinc carbonate, calcium carbonate, sodium carbonate, and a bicarbonate of magnesium, aluminum, iron, or zinc, or a combination of any of the previously listed options. In some embodiments of the composition of this disclosure, the putty A, the putty B, or both do not comprise a particulate material.

In some embodiments of the composition of this disclosure, the one or more additive material(s) of the putty A, the putty B, or both is a colorant, an antioxidant, an antibiotic, an antibacterial, an anti-infective, an active chemical hemostat, a steroid, calcium stearate, tocopheryl acetate, triacetin, a nonabsorbable plasticizer, other therapeutic agent(s), or any combination thereof. In some embodiments of the composition of this disclosure, the one or more additive material(s) of the putty A, the putty B, or both comprise a carbonate or bicarbonate selected from calcium carbonate, magnesium carbonate, aluminum carbonate, iron carbonate, zinc carbonate, calcium bicarbonate, sodium bicarbonate, embedded particles of bone, demineralized bone, bone morphogenetic protein, hydroxyapatite, calcium phosphate, siliconized calcium phosphate, aluminum carbonate, iron carbonate, zinc carbonate, calcium carbonate, sodium carbonate, and bicarbonate of magnesium, aluminum, iron, zinc, and combinations thereof. In some embodiments of the composition of this disclosure, the active chemical hemostat is a blood clot-inducing agent selected from a group consisting of prothrombin, thrombin, fibrinogen, fibrin, or any combination thereof. In some embodiments of the composition of this disclosure, the one or more additive material(s) of the putty A, the putty B, or both comprise starch, antimicrobial agent(s), colorant(s), x-ray opaque substance(s), and water. In some embodiments of the composition of this disclosure, the one or more additive material(s) of the putty A, the putty B, or both comprise a foaming agent (e.g., water). In some embodiments of the composition of this disclosure, the putty A, the putty B, or both do not comprise an additive material.

In some embodiments, the settable, nonabsorbable polyurethane, polyureaurethane, polyetherurethane or polyetherureaurethane compositions are polymers or prepolymers formed from the reaction of (i) a nondegradable polyaromatic isocyanate, preferably a diisocyanate or a polyisocyanate, and (ii) a nondegradable polyol and/or a polyamine, with the optional addition of (iii) a chain extender or crosslinker or curative. The terms “isocyanate” and “polyisocyanate” as used throughout herein, may be used interchangeably to refer to the polyaromatic isocyanates used in making the curable, nondegradable polyurethane, polyureaurethane, polyetherurethane or polyetherureaurethane compositions. The term “polyisocyanate” encompasses a chemical structure having two or more isocyanate groups. The term “polyaromatic” refers to the isocyanate groups residing on two or more aromatic rings. The term “polyol” encompasses a chemical structure having two or more hydroxyl groups. As used herein, the term “polyol” refers to both diols and polyols. Similarly, the term “polyamine” refers to a chemical structure having two or more amino groups present, and can be used to refer to both diamines and polyamines.

The polyaromatic isocyanates used to form the polyurethane, polyureaurethane, polyetherurethane or polyetherureaurethane compositions comprise no hydrolysable linkages bridging the aromatic rings. The term “polyaromatic isocyanates” as used herein is meant to distinguish from aromatic isocyanates having only a single aromatic ring, such as toluene diisocyanate. The isocyanate, polyol, and chain extender components, as well as other optional components are described in more detail below.

In some embodiments of the compositions of the disclosure, the putty A comprises a prepolymer formed from the reaction of excess polyisocyanate with the polyol/polyamine component. In some embodiments of the compositions of the disclosure, the putty B comprises a prepolymer formed from the reaction of excess polyol/polyamine component with the polyisocyanate component. In some embodiments of the compositions of the disclosure, the putty A comprises a prepolymer formed from the reaction of excess polyisocyanate with the polyol/polyamine component; and the putty B comprises a prepolymer formed from the reaction of excess polyol/polyamine component with the polyisocyanate component. In some embodiments of the compositions of the disclosure, the compositions described herein are formed by a process of combining a polyisocyanate prepolymer with a polyol or chain-extender and a catalyst, optionally with one or more particulate materials as described above, to form a poly(isocyanurate) composition, as shown below.

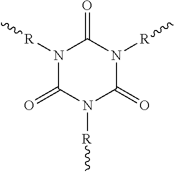

In another embodiment, the isocyanate prepolymer is combined with a polyol, water, and a catalyst, optionally with an osteoconductive filler, to form a poly(urethane-urea-isocyanurate) composition.

In some embodiments, the compositions comprise an ether-linked polyaromatic triisocyanate and a trimethylolpropane ethoxylated polyol, wherein the compositions are formed from the reaction of the polyaromatic polyisocyanate, one or more polyols and/or polyamines, and, optionally, a polyol and/or a polyamine as a chain extender. In some embodiments, the composition further comprises one or more of water, a carboxylic acid (e.g., benzoic acid, as foaming agent), a divalent or polyvalent metal salt, a metal carbonate or bicarbonate, or a phosphate (e.g., for osteoconductivity). The ether-linked triisocyanate monomer as described herein has the following structure:

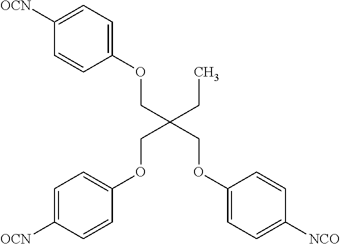

The trimethylolpropane ethoxylate polyol described herein has the following structure:

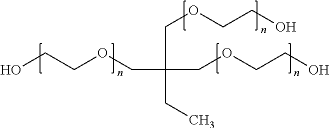

The settable nonabsorbable compositions can comprise constituent components (e.g., putties) comprising one or more di- or polyaromatic, di- or polyisocyanates containing no hydrolysable linkages within its structure. Hydrolysable polyurethanes can be fabricated utilizing a polyisocyanate that contains ester linkages. The polyisocyanate of the nonabsorbable compositions contains only ether linkages, which are resistant to hydrolysis at physiological conditions.

In some embodiments, the nonabsorbable polyurethane described herein is prepared from one or more aromatic isocyanates selected from the following the compounds:

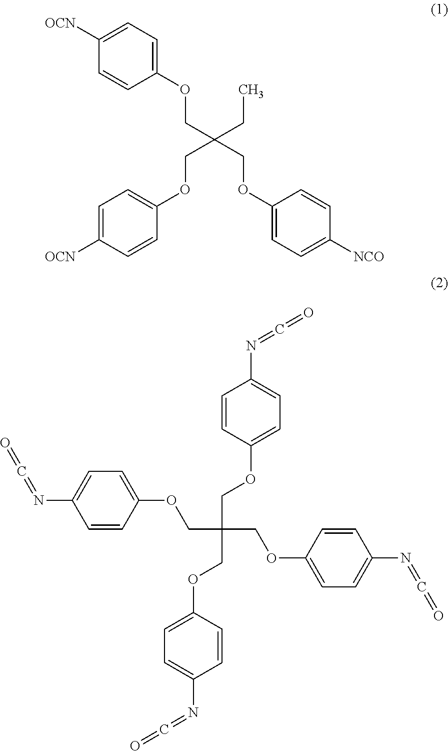

In some embodiments of the compositions comprising a set of at least two reactive putties, the constituent components (e.g., the first and at least the second putty or putty A or at least putty B) comprise any one or more of the di- or polyisocyanates disclosed herein. In certain embodiments, the polyisocyanate is an aromatic isocyanate, an aliphatic isocyanate, a cycloaliphatic isocyanate, or an adduct of an isocyanate.

### The Polyol/Polyamine Component

The settable nonabsorbable compositions can comprise constituent components (e.g., putties) comprising one or more diols/polyols and/or diamines/polyamines suitable for use in forming the polyurethane, polyureaurethane, polyetherurethane or polyetherureaurethane compositions, wherein the one or more diols/polyols and/or diamines/polyamines are nondegradable under physiological conditions.

The term “polyol” as used herein, refers to diols and polyols, unless indicated otherwise. In some embodiments, the compositions are prepared by combining an excess of the polyisocyanate component with the polyol/polyamine component. In some embodiments, the relative amounts of the polyisocyanate component and the polyol/polyamine component are calculated as the molar ratio of NCO groups of the polyisocyanate component (I) to the active hydroxyl/amino functional groups (H) found in the polyol and polyamine. In some embodiments, the ratio of polyisocyanate to polyol/polyamine (I:H) is at least 2:1. In some embodiments, the ratio is about 1.5:1, about 3:1, or about 4:1. In some embodiments, the ratio is about 5:1, about 8:1, about 10:1, about 20:1, or about 50:1.

In some embodiments of the compositions, the polyol/polyamine component is present in a polyisocyanate prepolymer in an amount ranging from about 5 wt % to about 50 wt % of the prepolymer. In some embodiments of the compositions, the polyol/polyamine component is present in an amount ranging from about 5 wt % to about 10 wt %, about 10 wt % to about 20 wt %, about 20 wt % to about 35 wt %, about 25 wt % to about 40 wt %, or from about 35 wt % to about 50 wt % of the prepolymer.

Non-limiting examples of the one or more polyols suitable for use in the compositions include biocompatible naturally occurring polyols, synthetic polyols, and mixtures thereof. In some embodiments, the one or more polyols of compositions comprise at least one ether group.

In some embodiments, the one or more polyols of compositions comprise 2-4 ether groups or 5-10 ether groups. In some embodiments, the one or more polyols of compositions comprise two or more hydroxyl groups. Non-limiting examples of the one or more polyols suitable for use in the compositions include trimethylolpropane ethoxylate, triethanolamine, tetrakis(2-hydroxypropyl)ethylenediamine, tetrakis(2-hydroxyethyl)ethylenediamine, butanediol, poly(propylene glycol), isosorbide, or polycarbonate diols.

In some embodiments of the compositions, a polyisocyanate prepolymer component is combined with a polyamine component to form a poly(urethane-urea). In some embodiments of the compositions, the polyamine component is a primary or secondary diamine, or a hindered amine. Non-limiting examples of suitable polyamines of the compositions comprise hindered diamine (e.g., isophorone diamine, or IPDA), 1,4-cyclohexyl diamine, 1,3-pentane diamine, or aliphatic secondary diamines, or a combination thereof. In some embodiments of the compositions, the polyamine component comprises aliphatic diamines and/or cycloaliphatic diamines.

### The Polyol/Polyamine Component for Multi-Putty Compositions

Non-limiting examples of the one or more polyols suitable for use in the multi-putty (at least two putties) compositions include any of the biocompatible, naturally occurring polyols, synthetic polyols, and mixtures thereof disclosed herein. In certain embodiments, the one or more polyols comprise at least one ether group. In certain embodiments, the one or more polyols comprise 2-4 ether groups or 5-10 ether groups. In certain embodiments, the one or more polyols have at least two hydroxyl groups. In certain embodiments, the one or more polyols have three or more hydroxyl groups.

The polyurethane, polyureaurethane, polyetherurethane or polyetherureaurethane reactants/components and reactions described herein, can be applied directly to the multi-putty compositions (at least two putties). In some embodiments, the compositions are produced by mixing a first putty composition (e.g., “Component/Putty A”), which comprises one or more reactants capable of participating in chemical reactions with one or more reactants present in a second putty composition (e.g., “Component/Putty B”), and, optionally, with a reactive third, fourth, fifth or more reactants in any number of additional putties (e.g. any number as deemed necessary or useful to produce a product that is harder, less flowable, and/or more cohesive than the individual component putties). In some embodiments of the compositions the individual components/putties can be formed by preparing a suspension of particulates within a liquid. In some embodiments of the compositions the individual components/putties can comprise one or more moldable solids (e.g., a wax-like material, a particulate solid, or a modeling clay combined with a moldable solid and/or a moldable solid and a liquid).

Non-limiting examples of osteoconductive additives that can be included in the compositions include carbonate (e.g., calcium carbonate, magnesium carbonate, aluminum carbonate, iron carbonate, zinc carbonate, calcium bicarbonate, and sodium bicarbonate), bone (e.g., demineralized bone matrix, bone morphogenetic protein, allograft bone, and/or autologous bone), calcium phosphate, siliconized calcium phosphate, substituted calcium phosphates (e.g., with magnesium, strontium, or silicate), calcium pyrophosphate glass, calcium sulfate, tricalcium phosphate (e.g., p-tricalcium phosphate), or any combination thereof.

In some embodiments, the one or more constituent components/putties of the compositions comprise an optional additive material in an amount of from about 0.01 wt % to about 80 wt % of the composition. In some embodiments, the one or more constituent components/putties of the compositions comprise the additive material in an amount of 5-10 wt %, 10-20 wt %, 25-35 wt %, 20-40 wt %, 35-55 wt %, 50-70 wt %, 65-80 wt %, or more than 80 wt % of the composition. In some embodiments, the optional additive is in nano-scale sizes. In some embodiments, the optional additive is in micron or millimeter sizes, or mixtures thereof.

### Other Optional Additives

In some embodiments, the compositions comprise one or more “cell openers”. Non-limiting examples of cell openers as described herein include ORTOGEL501 (Goldschmidt) and X-AIR (Specialty Polymers & Services). In some embodiments, the cell openers are present in an amount from about 0.1 wt % to about 5 wt % of the composition. In some embodiments, the cell openers are present in amounts of about 1 wt % to about 2 wt %. In some embodiments, the cell openers are present in amounts of about 1 wt % to about 3 wt % of the composition.

In some embodiments, the compositions comprise one or more antibiotics. Non-limiting examples of antibiotics as described herein include beta-lactam antibiotics such as tobramycin, subclasses Penicillins (examples: penicillin G, methicillin, oxacillin, ampicillin, amoxicillin), Cephalosporins, Glycopeptides (example vancomycin), Carbapenems (examples imipenem and meropenem), Polymyxin and Bacitracins (example bacitracin, neomycin) or Lipopeptides (example daptomycin), Protein synthesis inhibitors such as subclasses Aminoglycosides (example gentamicin, streptomycin, kanamycin), Tetracyclines (examples tetracycline, doxycycline, minocycline, and tigecycline), Oxazilodinone (linezolid), Peptidyl transferases (example Chloramphenicol), Macrolides (examples erythromycin, azithromycin, telithromycin), Lincosamides (examples clindamycin), and Streptogramins (example prisintamycin), DNA synthesis inhibitors such as metronidazole and subclass Fluoroquinolones (examples ciprofloxacin, norfloxacin, morifloxacin), RNA synthesis inhibitors such as rifampin, Mycolic acid synthesis inhibitors such as isoniazid, and Folic acid synthesis inhibitors such as Trimethoprim and subclass Sulfonamides (examples sulfamethoxazole, sulfadoxin). In some embodiments, the antibiotic is present in an amount ranging from about 0.01 wt % to about 8 wt % of the composition. In certain embodiments, gentamicin is present at a concentration from about 10 mg/cc to about 200 mg/cc of the composition. In certain embodiments, vancomycin is present from about 40 mg/cc to about 600 mg/cc of the composition. In certain embodiments, minocycline is present from about 5 mg/cc to about 200 mg/cc of the composition. In certain embodiments, rifampin is present from about 10 mg/cc to about 300 mg/cc of the composition.

In some embodiments, the compositions comprise one or more local anesthetics. Non-limiting examples of local anesthetics include lidocaine, bupivacaine, tetracaine, and ropivacaine, including the freebases, their salts, and derivatives thereof.

In some embodiments, the compositions comprise one or more antioxidants. Non-limiting examples of suitable antioxidants include Vitamin E acetate, IRGANOX 1010 and IRGANOX 1035 (Ciba Geigy), and CYANOX 1790 and CYANOX 2777 (Cytec Industries). In some embodiments, the antioxidant is present in an amount ranging from about 0.01 wt % to about 5 wt % of the composition.

In some embodiments, the compositions comprise a steroid-based compound, such as an intracellular messenger, to modulate the rate of bone growth. In some embodiments, the compositions comprise progenitor cells.

### Particulate Materials

Both the putty and non-putty compositions may contain optional particulate materials. In one embodiment, the particulate material is an osteoconductive material. In certain embodiments, the particulate material supports cell attachment at the application site. In certain embodiments, the mean particle size of the optional particulate material is in the micron or submicron range. In one embodiment, the mean particle size is from 0.001 to 0.100 μm, from 0.100 to 1 μm, from 1 to 5 μm, from 5 to 500 μm, or from 500 to 1000 μm.

In some embodiments, the particulate material is a carbonate or bicarbonate (e.g., calcium carbonate, magnesium carbonate, aluminum carbonate, iron carbonate, zinc carbonate, calcium bicarbonate, sodium bicarbonate, or any combination thereof). In some embodiments, the particulate material is bone (e.g., demineralized bone matrix, bone morphogenetic protein, allograft bone, and/or autologous bone), calcium phosphate, siliconized calcium phosphate, substituted calcium phosphates (e.g., with magnesium, strontium, or silicate), calcium pyrophosphate, hydroxyapatite, poly(methyl methacrylate), glass-ionomer, calcium sulfate, tricalcium phosphate (e.g., β-tricalcium phosphate), or any combination thereof. In some embodiments, the particulate material is a polyether ether ketone (PEEK), REPLACE (Cortek, Inc.), and EX-PANCEL (Akzo Nobel). In some embodiments, the particulate material is a ceramic, such as substituted calcium phosphates (e.g., silicate, strontium, or magnesium substitution), or a glass, such as bioglass. In some embodiments, the particulate material is one or more of calcium sulfate, calcium phosphosilicate, sodium phosphate, calcium aluminate, calcium phosphate, hydroxyapatite, demineralized bone matrix, or mineralized bone. In some embodiments, the particulate material, when present, may comprise any one or more of the materials listed in the embodiments above. In one embodiment, the particulate material, if present in the composition, does not comprise calcium carbonate.

In some embodiments, the compositions or the constituent components/putties of the compositions comprise the particulate material in an amount ranging from 0.01 to 10 wt % of the composition. In some embodiments, the compositions or the constituent components/putties of the compositions comprise the particulate material in an amount ranging from 0.10 to 10 wt %, 1 to 10 wt %, or 5 to 10 wt %. In some embodiments, the compositions or the constituent components/putties of the compositions comprise the particulate material in an amount ranging from 10 to 20 wt %, 20 to 30 wt %, 30 to 40 wt %, 40 to 50 wt %, 50 to 60 wt %, 60 to 70 wt %, or 70 to 80 wt % of the composition.

### Foaming Agents

In some embodiments, the compositions or the constituent components/putties of the compositions comprise an optional foaming agent to, for example, modulate pore size. In some embodiments, the foaming agent is carboxylic acids, wherein the carboxylic acids react with isocyanates present to form carbon dioxide (and the corresponding amide). Non-limiting examples of carboxylic acids that can be used in this manner are benzoic acid, malic acid, and succinic acid. In certain embodiments, some embodiments, the compositions or the constituent components/putties of the compositions are formed by a process of combining a polyol and/or polyamine, polyisocyanate, and a carboxylic acid. In some embodiments, the carboxylic acid does not contain water. In some embodiments, the compositions or the constituent components/putties of the compositions formed with carboxylic acid do not contain added water. In some embodiments, the compositions or the constituent components/putties of the compositions comprise albumen as a foaming agent, with or without sodium alginate. In another embodiment, the compositions or the constituent components/putties of the compositions comprise hydrogen peroxide as a foaming agent.

### Catalyst Component

In some embodiments, the compositions or the constituent components/putties of the compositions comprise an optional catalyst (e.g., added to the polyol that is combined with the isocyanate to form the compositions). In certain embodiments, the compositions or the constituent components/putties of the compositions comprise at least one catalyst in an amount sufficient to ensure that the polymerization reactions have proceeded to completion before the compositions are placed within the body of a subject. Non-limiting examples of catalysts include a tertiary amine (e.g., DABCO 33LV, Air Products, Inc.) and organometallic compounds, such as stannous octoate and dibutyl tin dilaurate. In some embodiments, the catalyst may remain in the compositions of the disclosure, after its formulation and curing, for example, as a monomer that is present in the matrix of the solidified form of the composition. A non-limiting example of such a catalyst is N,N,N′-Tri(2-hydroxylpropyl)-N′-hydroxyethyl ethylene diamine (POLY-Q-40-800, Arch Chemicals, Inc.).

In some embodiments, the compositions or the constituent components/putties of the compositions comprise a catalyst in an amount ranging from about 0.05 wt % to about 0.5 wt % of the polyol. In some embodiments, the compositions or the constituent components/putties of the compositions comprise a catalyst in an amount ranging from about 0.15 wt % to about 0.4 wt % of the polyol.

### Optional Surfactant Component

In some embodiments, the compositions or the constituent components/putties of the compositions comprise an optional surfactant in order to control the porosity of the composition including the size and/or shape of the pores within the composition. Non-limiting examples of suitable surfactants include DABCO DC 193 and DABCO DC 5241 (Air Products, Inc.), MAXEMUL 6106 (Unigema), and silicone surfactants (e.g., those available from Struktol Corp.).

### Radiotransparent/Radiopaque Component

In some embodiments, the compositions or the constituent components/putties of the compositions comprise an optional filler, wherein the filler is radiopaque (e.g., calcium phosphate granules) and imparts radiopacity to the hardened composition. In some embodiments, the compositions or the constituent components/putties of the compositions comprise an optional radiotransparent and/or a radiopaque substance. Non-limiting examples of a radiotransparent substance include air, nitrogen gas, carbon dioxide, and oxygen gas. Non-limiting examples of a radiopaque substance include ceramic particles (e.g., calcium phosphate), barium sulfate (BaSO₄), and zirconium dioxide (ZrO₂). Examples of commercially available radiopaque substances include LIPIODOL, HYPAQUE, and OMNIPAQUE. In some embodiments, the compositions or the constituent components/putties of the compositions comprise a radiotransparent and/or radiopaque substances in amounts of about 5 wt % to about 30 wt % of the composition, and, in certain embodiments, from about 10 wt % to about 20 wt % of the composition.

### Protein Component

In some embodiments, the compositions or the constituent components/putties of the compositions optionally comprise one or more bioactive proteins, pep-tides, or polypeptides.

In some embodiments, the one or more bioactive proteins, peptides, or polypeptides is active in the stimulation of bone growth. Non-limiting examples of suitable proteins include collagen, OP1 (Stryker Homedica), INFUSE (Medtronic Corp.), or any recombinant bone morphogenic protein. In some embodiments, the one or more bioactive proteins, peptides, or polypeptides is non-reactive with the other components of the composition, allowing it to be included at any point during the formulating process. In some embodiments, the one or more peptides is not incorporated into the polymer backbone of the compositions or the constituent components/putties of the compositions, but instead is either embedded in the polymer matrix, dispersed in the composition, or adherent to the surface of the compositions or the constituent components/putties of the compositions.

The one or more bioactive proteins, peptides, or polypeptides may be incorporated within the compositions or the constituent components/putties of the compositions, for example, by inclusion in the process of combining the polyisocyanate component and the polyol/polyamine component. In some embodiments of the compositions or the constituent components/putties of the compositions, the one or more bioactive proteins, peptides, or polypeptides is dispersed throughout the composition or the constituent components/putties of the compositions. In some embodiments of the compositions or the constituent components/putties of the compositions, the one or more bioactive proteins, peptides, or polypeptides is added after combination of the other components. In some embodiments of the compositions or the constituent components/putties of the compositions, the one or more bioactive proteins, peptides, or polypeptides is added about 10 to 45 minutes after combination of the other components. In some embodiments, the one or more bioactive proteins, peptides, or polypeptides adheres to an outer surface of the composition.

### Optional Light- or Photo-Initiators

In some embodiments, the compositions or the constituent components/putties of the compositions comprise light- or photo-initiators. Non-limiting examples of suitable light- or photo-initiators include 24650-42-8 (Loctite Corp.). In some embodiments, the light- or photo-initiators are included in compositions or the constituent components/putties of the compositions are made from unsaturated components (e.g., isocyanate prepolymers having one or more double bonds or polyols having polarized double bonds). In some embodiments, a photo- or light-initiator is incorporated into the compositions or the constituent components/putties of the compositions by combining with a liquid component (e.g., a polyisocyanate, a polyol, a polyamine, a chain extender, or a crosslinker). In certain embodiments, the compositions or the constituent components/putties of the compositions comprising a photo- or light-initiator, solidify at an accelerated rate (e.g., in the range of about 1 to 5 minutes or 1 to 10 minutes) after exposure to a suitable energy source (e.g., a suitable light source).

### Other Optional Additives

In certain embodiments, the compositions or the constituent components/putties of the compositions optionally comprise one or more “cell openers”. Non-limiting examples of cell openers include ORTOGEL501 (Goldschmidt) and X-AIR (Specialty Polymer & Services). In certain embodiments, the compositions or the constituent components/putties of the compositions comprise one or more cell openers in an amount ranging from about 0.1 wt % to about 5 wt % of the composition. In some embodiments, the compositions or the constituent components/putties of the compositions comprise one or more cell openers in an amount ranging from about 1 wt % to about 2 wt % or about 1 wt % to about 3 wt % of the composition.

In some embodiments, the compositions or the constituent components/putties of the compositions optionally comprise one or more antibiotics. Non-limiting examples of antibiotics as described herein include beta-lactam antibiotics such as tobramycin, subclasses Penicillins (examples: penicillin G, methicillin, oxacillin, ampicillin, amoxicillin), Cephalosporins, Glycopeptides (example vancomycin), Carbapenems (examples imipenem and meropenem), Polymyxin and Bacitracins (example bacitracin, neomycin) or Lipopeptides (example daptomycin), Protein synthesis inhibitors such as subclasses Aminoglycosides (example gentamicin, streptomycin, kanamycin), Tetracyclines (examples tetracycline, doxycycline, minocycline, and tigecycline), Oxazilodinone (linezolid), Peptidyl transferases (example Chloramphenicol), Macrolides (examples erythromycin, azithromycin, telithromycin), Lincosamides (examples clindamycin), and Streptogramins (example prisintamycin), DNA synthesis inhibitors such as metronidazole and subclass Fluoroquinolones (examples ciprofloxacin, norfloxacin, morifloxacin), RNA synthesis inhibitors such as rifampin, Mycolic acid synthesis inhibitors such as isoniazid, and Folic acid synthesis inhibitors such as Trimethoprim and subclass Sulfonamides (examples sulfamethoxazole, sulfadoxin). In some embodiments, the antibiotic is present in an amount ranging from about 0.01 wt % to about 8 wt % of the composition. In certain embodiments, gentamicin is present at a concentration from about 10 mg/cc to about 200 mg/cc of the composition. In certain embodiments, vancomycin is present from about 40 mg/cc to about 600 mg/cc of the composition. In certain embodiments, minocycline is present from about 5 mg/cc to about 200 mg/cc of the composition. In certain embodiments, rifampin is present from about 10 mg/cc to about 300 mg/cc of the composition.

In some embodiments, the compositions or the constituent components/putties of the compositions optionally comprise one or more local anesthetics or analgesics. Non-limiting examples of local anesthetics or analgesics include lidocaine, bupivacaine, tetracaine, and ropivacaine. Further non-limiting examples of local anesthetics or analgesics include benzocaine and fentanyl (a potent synthetic opioid).

In some embodiments, the compositions or the constituent components/putties of the compositions optionally comprise one or more anti-inflammatory substances, such as the nonspecific ibuprofen and/or aspirin, or the COX-2 specific inhibitors, such as rofecoxib and celeboxib.

In some embodiments, the compositions or the constituent components/putties of the compositions optionally comprise one or more antioxidants. Non-limiting examples of suitable antioxidants include IRGANOX 1010 and IRGANOX 1035 (Ciba Geigy), and CY-ANOX 1790 and CYANOX 2777 (Cytec Industries). In some embodiments, the compositions or the constituent components/putties of the compositions comprise one or more antioxidants in an amount ranging from about 0.01 wt % to about 0.5 wt % of the composition.

In some embodiments the compositions or the constituent components/putties of the compositions further comprise a colorant. Non-limiting examples of suitable colorants include gentian violet, D&C Violet #2, and D&C Green #6.

In some embodiments, the compositions or the constituent components/putties of the compositions comprise a steroid-based compound. Such an intracellular messenger may optionally be included in the compositions to modulate the rate of bone growth. In some embodiments, progenitor cells optionally may be included in the compositions.

The nonabsorbable putty composition (nonabsorbable composition) can be formed by mixing at least two putties. In some embodiments, the nonabsorbable composition comprises 5% to 35% of a polyisocyanate component, 2.5% to 42.5% of a polyol/polyamine component, 30% to 90% of one or more particulate matter(s), and 0% to 6.5% of one or more additive material(s), based upon the weight of the composition.

In some embodiments, the nonabsorbable composition comprises 5% to 35% (e.g., 5% to 10%, 10% to 15%, 15% to 20%, 20% to 25%, 25% to 30% or 30% to 35%) of a polyisocyanate component, based upon the weight of the composition. In some embodiments, the nonabsorbable composition comprises 2.5% to 42.5% (e.g., 2.5% to 5%, 5% to 7.5%, 7.5% to 10%, 10% to 12.5%, 12.5% to 15%, 15% to 17.5%, 17.5% to 20%, 20% to 22.5%, 22.5% to 25%, 25% to 27.5%, 27.5% to 30%, 30% to 32.5%, 32.5% to 35%, 35% to 37.5%, 37.5% to 40%, or 40% to 42.5%) of a polyol/polyamine component, based upon the weight of the composition. In some embodiments, the nonabsorbable composition comprises 30% to 90% (e.g., 30% to 35%, 35% to 40%, 40% to 45%, 45% to 50%, 50% to 55%, 55% to 60%, 60% to 65%, 65% to 70%, 70% to 75%, 75% to 80%, 80% to 85%, or 85% to 90°/%) of one or more particulate matter(s), based upon the weight of the composition. In some embodiments, the nonabsorbable composition comprises 0% to 6.5% (e.g., 0% to 2%, 2% to 4% or 4% to 6.5%) of one or more additive material(s), based upon the weight of the composition.

In some embodiments, the nonabsorbable composition comprises 12.5% to 30% of a polyisocyanate component, 15% to 35% of a polyol/polyamine component, and 35% to 72.5% of one or more particulate matter(s), based upon the weight of the composition. In some embodiments, the nonabsorbable composition comprises 12.5% to 30% of a polyisocyanate component (e.g., 12.5% to 15%, 15% to 17.5%, 17.5% to 20%, 20% to 22.5%, 22.5% to 25%, 25% to 27.5%, or 27.5% to 30%), based upon the weight of the composition. In some embodiments, the nonabsorbable composition comprises 15% to 35% of a polyol/polyamine component (e.g., 15% to 17.5%, 17.5% to 20%, 20% to 22.5%, 22.5% to 25%, 25% to 27.5%, 27.5% to 30%, 30% to 32.5%, or 32.5% to 35%), based upon the weight of the composition. In some embodiments, the nonabsorbable composition comprises 35% to 72.5% of one or more particulate matter(s) (e.g., 35% to 37.5%, 37.5% to 40%, 40% to 42.5%, 42.5% to 45%, 45% to 47.5%, 47.5% to 50%, 50% to 52.5%, 52.5% to 55%, 55% to 57.5%, 57.5% to 60%, 60% to 62.5%, 62.5% to 65%, 65% to 67.5%, 67.5% to 70%, or 70% to 72.5%), based upon the weight of the composition.

In some embodiments, the nonabsorbable composition comprises 8.5% to 22.5% of a polyisocyanate component, 8.5% to 14% of a polyol/polyamine component, and 65% to 85% of one or more particulate matter(s), based upon the weight of the composition. In some embodiments, the nonabsorbable composition comprises 8.5% to 22.5% (e.g., 8.5% to 11%, 110% to 13.5%, 13.5% to 16%, 16% to 18.5%, 18.5% to 21%) of a polyisocyanate component, based upon the weight of the composition. In some embodiments, the nonabsorbable composition comprises 8.5% to 14% (e.g., 8.5% to 10%, 12% to 13%, or 13% to 14%) of a polyol/polyamine component, based upon the weight of the composition. In some embodiments, the nonabsorbable composition comprises 65% to 85% (e.g., 65% to 70%, 70% to 75%, 75% to 80%, or 80% to 85%) of one or more particulate matter(s), based upon the weight of the composition.

In some embodiments, the nonabsorbable composition comprises 13.5% to 25% of a polyisocyanate component, 4% to 20% of a polyol/polyamine component, and 55% to 78.5% of one or more particulate matter(s), based upon the weight of the composition. In some embodiments, the nonabsorbable composition comprises 13.5% to 25% (e.g., 13.5% to 15%, 15% to 17.5%, 17.5% to 20%, 20% to 22.5%, or 22.55 to 25%) of a polyisocyanate component, based upon the weight of the composition. In some embodiments, the nonabsorbable composition comprises 4% to 20% (e.g., 4% to 8%, 8% to 12%, 12% to 16%, or 16% to 20%) of a polyol/polyamine component, based upon the weight of the composition. In some embodiments, the nonabsorbable composition comprises 55% to 78.5% (e.g. 55% to 57.5%, 57.5% to 60%, 60% to 62.5%, 62.5% to 65%, 65% to 67.5%, 67.5% to 70%, 70% to 72.5%, 72.5% to 75%, or 75% to 78.5%) of one or more particulate matter(s), based upon the weight of the composition.

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Unless indicated otherwise, all percentages by are percentages by weight, parts are parts by weight, temperature is in degrees Celsius, and pressure is at or near atmospheric. There are numerous variations and combinations of reaction conditions (e.g., component concentrations, desired solvents, solvent mixtures, temperatures, pressures, and other reaction ranges and conditions) that can be used to optimize the product purity and yield obtained from the described process. Only reasonable and routine experimentation will be required to optimize such process conditions.

What is claimed is:

1. A method of forming a molded composition for bone repair or reconstruction, the method comprising:
determining an implant shape based on a shape of an implant site of a patient and a surgical procedure being performed on the patient;
selecting a mold based on the implant shape;
molding a settable composition using the mold to form a molded composition; and
implanting the molded composition in the patient.

2. The method of claim 1, wherein the settable composition is absorbable.

3. The method of claim 1, wherein the settable composition is non-absorbable.

4. The method of claim 1, further comprising:
forming, by combining a set of at least two components, the settable composition.

5. The method of claim 4, wherein the set of at least two components are selected from a group consisting of polyurethane, polyureaurethane, polyetherurethane, and polyetherureaurethane.

6. The method of claim 4, wherein:
a first component of the set of at least two components comprises a prepolymer of a polyisocyanate and one or more polyisocyanates, and
a second component of the set of at least two components comprises a prepolymer of a polyisocyanate and at least one polyol and/or polyamine.

7. The method of claim 1, further comprising:
processing the molded composition to match the implant shape.

8. The method of claim 7, wherein processing the molded composition comprises at least one of:
manipulating the molded composition at least one of by hand or by a processing tool to cause the molded composition to match the shape of the implant site; and
removing flash from the molded composition.

9. The method of claim 1,
wherein the molded composition includes at least two pieces,
wherein processing the molded composition comprises:
joining the at least two pieces of the molded composition via corresponding guides; and
removing the corresponding guides from the molded composition.

10. The method of claim 1, wherein forming the molded composition includes extruding the settable composition through a mold cavity of the mold.

11. The method of claim 1, wherein the molded composition has at least one of a cone shape, a spherical shape, a cylindrical shape, a half-spherical shape, a crescent moon shape, a rectangular shape, boat-shape, a high aspect ratio cylindrical shape, a half-moon shape, a circular shape, a ring shape, a dome shape, or a square shape.

12. A molded composition for bone repair formed by a process comprising the steps of:
determining an implant shape based on a shape of an implant site of a patient and a surgical procedure being performed on the patient;
selecting a mold based on the implant shape;
molding a settable composition using the mold to form a molded composition; and
implanting the molded composition in the patient.

13. The process of claim 12 where in the settable composition is absorbable.

14. The process of claim 12, wherein the settable composition is non-absorbable.

15. The process of claim 12, further comprising:
forming, by combining a set of at least two components, the settable composition.

16. The process of claim 15, wherein the set of at least two components are selected from a group consisting of polyurethane, polyureaurethane, polyetherurethane and polyetherureaurethane.

17. The process of claim 15, wherein:
a first component of the set of at least two components comprises a prepolymer of a polyisocyanate and one or more polyisocyanates; and
a second component of the set of at least two components comprises a prepolymer of a polyisocyanate and a polyol.

18. The process of claim 12, further comprising:
processing the molded composition to match the shape of the implant site.

19. The process of claim 18, wherein:
the molded composition includes at least two pieces; and
processing the molded composition comprises:
joining the at least two pieces of the molded composition via corresponding guides; and
removing from the molded composition the corresponding guides.

20. The process of claim 18, wherein processing the molded composition comprises:
manipulating the molded composition to match the shape of the implant site.

21. The process of claim 12, wherein the molded composition has at least one of a cone shape, a spherical shape, a cylindrical shape, a half-spherical shape, a crescent moon shape, a rectangular shape, boat-shape, a high aspect ratio cylindrical shape, a half-moon shape, a circular shape, a ring shape, a dome shape, or a square shape.

22. A kit for use in tissue repair or reconstruction, the kit comprising:
a settable composition; and
at least one mold structured to mold the settable composition into a molded composition having a shape corresponding to a shape of an implant site of a patient.

23. The kit of claim 22, wherein the settable composition comprises a first component and a second component.

24. The kit of claim 22, further comprising:
at least one processing tool configured to assist a user in further shaping the molded composition into a shape corresponding to the shape of the implant site.

25. The kit of claim 22, wherein the molded composition has at least one of a cone shape, a spherical shape, a cylindrical shape, a half-spherical shape, a crescent moon shape, a rectangular shape, boat-shape, a high aspect ratio cylindrical shape, a half-moon shape, a circular shape, a ring shape, a dome shape, or a square shape.
